(19) United States
(12) Patent Application Publication
(10) Pub. No.: US 2023/0272490 A1
(43) Pub. Date: Aug. 31, 2023

(54) NASAL MICROBIOME BIOMARKERS FOR PREDICTING THE ONSET OF BOVINE RESPIRATORY DISEASE AND TREATING THE SAME

(71) Applicant: THE BOARD OF TRUSTEES OF THE UNIVERSITY OF ARKANSAS, Little Rock, AR (US)

(72) Inventors: Jiangchao ZHAO, Fayetteville, AR (US); Jianmin CHAI, Fayetteville, AR (US); Elizabeth B. KEGLEY, Springdale, AR (US); Jeremy POWELL, Lowell, AR (US)

(21) Appl. No.: 18/020,241

(22) PCT Filed: Aug. 9, 2021
(86) PCT No.: PCT/US2021/045223

§ 371 Date: Feb. 7, 2023

Related U.S. Application Data
(60) Provisional application No. 63/062,502, filed on Aug. 7, 2020.

(51) Int. Cl. C12Q 1/689; A61D 99/00

(52) U.S. Cl. CPC C12Q 1/689; A61D 99/00; C12Q 2600/106; A61B 10/0051

(57) ABSTRACT

Sets of bacterial features were identified in the nostrils, nasopharynx, and lungs of cows that can be used to predict the likelihood that a cow will develop bovine respiratory disease (BRD) or to diagnose BRD. The present invention provides methods and kits for selecting cows to treat for BRD based on the levels of these biomarkers in the respiratory microbiome. Using these methods and kits, producers may selectively treat cows deemed to be at risk for BRD, saving money and decreasing antibiotic use.

### CROSS-REFERENCE TO RELATED APPLICATIONS

This application claims priority to U.S. Provisional Application No. 63/062,502 filed on Aug. 7, 2020, the contents of which are incorporated by reference in their entireties.

### STATEMENT REGARDING FEDERALLY SPONSORED RESEARCH

This invention was made with government support under grant number NA/NI8AHDRXXXXG017 awarded by the United States Department of Agriculture, National Institute of Food and Agriculture. The government has certain rights in the invention.

### SEQUENCE LISTING

A Sequence Listing accompanies this application and is submitted as an ASCII text file of the sequence listing named “169946_00622_ST25.txt” which is 40,867 bytes in size and was created on Jul. 30, 2021. The sequence listing is electronically submitted via EFS-Web with the application and is incorporated herein by reference in its entirety.

### INTRODUCTION

Bovine respiratory disease (BRD) is a leading cause of morbidity and mortality in cattle [1]. Calves are weaned between 6 and 8 months of age, at which time they are usually sold and moved to a new location through local auction markets. The physical and psychological stress associated with weaning and transporting these calves increases their susceptibility to infection.

The beef industry's contribution to the U.S. economy is colossal. In 2016 alone, the United States produced 30.2 million head of beef calves with farm gate receipts estimated to be in excess of $44 billion. In this country, BRD accounts for 70 to 80% of all feedlot morbidity and to 50% of feedlot mortality. Not only does this disease result in increased medication costs, but morbid cattle also grow slower, are less efficient in converting feed to gains, and have carcasses of a lower quality grade after slaughter. Despite the large economic burden this disease creates for the beef industry, little progress has been made with respect to prognosis, as clinical diagnosis of this disease is still based on subjective observations.

Further, despite recent advances in vaccine and antimicrobial technologies, health outcomes of BRD have not improved, as morbidity and mortality rates have not decreased over the last 20 years [2]. Current therapeutic options for controlling and treating BRD include vaccinating against viruses that initiate the disease and providing antimicrobial treatment to control secondary bacterial infections. Treatment usually relies on the application of broad-spectrum antibiotics, and may thus contribute to the generation and spread of antibiotic resistance. With an estimated cost of $23.60 per antimicrobial treatment, the cost of treating BRD equated to over $227 million in 2011 alone. Yet, in feedlots, 4% of cattle treated for BRD still die from this disease and another 2.3% of treated cattle become chronically morbid and are sold prematurely at a reduced price.

Pressure from consumers for a prohibition on the use of antimicrobials in livestock continues to increase. In response, there is growing interest in using preventive methods and alternative therapies, such as probiotics, to treat BRD. A better understanding of BRD pathogen ecology and the complex interplay of microbes within the respiratory tracts of sick and healthy cattle would facilitate the development of such therapies. Further, a better understanding of BRD could accelerate the diagnosis of BRD, such that antibiotic therapies can be targeted to high-risk calves.

### SUMMARY

In a first aspect, the present invention provides methods for selecting cows to treat for bovine respiratory disease (BRD). The methods include collecting a nasal swab, nasopharyngeal swab, or bronchoalveolar lavage sample from a cow, measuring the level of at least one biomarker associated with a bacterium, and analyzing the abundance of the biomarker to determine whether to treat the cow. The inventors demonstrate herein that (1) the presence, absence, or level of a first set of bacteria in the respiratory microbiome of a cow are indicative of the likelihood that a cow will develop BRD while (2) the presence, absence, or level of a second set of bacteria indicate that a cow has BRD.

For the nasal swab samples, the level of a biomarker associated with a bacterium of a species selected from Fusobacterium mortiferum, Prevotella stercorea, Bacteroides vulgatus, Prevotella oris, Clostridium saudiense, Lactobacillus plantarum, Bacteroides uniformis, [Clostridium] clostridioforme, Lactobacillus mucosae, Gemmiger formicilis, Prevotella copri, Terrisporobacter petrolearius, Blautia obeum, [Clostridium] scindens, Lactobacillus caviae, Ruminococcus lactaris, Catenibacterium mitsuokai, Kineothrix alysoides, and Streptococcus pasteurianus is indicative of the likelihood that a cow will develop BRD, while the level of a biomarker associated with a bacterium of a species selected from Clostridium butyricum, Lactobacillus gasseri, Holdemanella biformis, Clostridium saudiense, Catenibacterium mitsuokai, Faecalibacterium prausnitzii, Prevotella stercorea, Ruminococcus faecis, Prevotella copri, Fusicatenibacter saccharivorans, Gemmiger formicilis, [Eubacterium] eligens, Butyricicoccus pullicaecorum, Blautia wexlerae, Ruminiclostridium cellobioparum, Massiliprevotella massiliensis, Prevotellamassilia timonensis, and Lactobacillus mucosae is indicative of whether a cow has BRD.

For the nasopharyngeal swab samples, the level of a biomarker associated with a bacterium of a species selected from Streptococcus uberis, Salmonella enterica, Kingella negevensis, Prevotella copri, Streptococcus pluranimalium, Holdemanella biformis, Veillonella dispar, Collinsella aerofaciens, Ruminococcus bromii, Prevotella oris, Fournierella massiliensis, Bacteroides plebeius, Lactobacillus mucosae, and Alistipes finegoldii is indicative of the likelihood that a cow will develop BRD, while the level of a biomarker associated with a bacterium of a species selected from Ruminococcus faecis, Prevotella copri, Gemmiger formicilis, Butyricicoccus pullicaecorum, Blautia wexlerae, Faecalibacterium prausnitzii, Dorea formicigenerans, Blautia obeum, Bacteroides fragilis, Coprococcus comes, Blautia luti, Dorea longicatena, [Ruminococcus] gnavus, [Eubacterium] hallii, Schaalia cardiffensis, Prevotella stercorea, and Clostridium perfringens is indicative of whether a cow has BRD.

For the bronchoalveolar lavage samples, the level of a biomarker associated with a bacterium of a species selected from Mycoplasma dispar, Mannheimia haemolytica, Moraxella caviae, Micrococcus luteus, Massilia agri, Terrimonas lutea, Alkalibacter saccharofermentans, [Clostridium] glycyrrhizinilyticum, Flavobacterium acidificum, Alistipes putredinis, Collinsella aerofaciens, Solibacillus isronensis, and Monoglobus pectinilyticus is indicative of the likelihood that a cow will develop BRD, while the level of a biomarker associated with a bacterium of a species selected from Caldalkalibacillus thermarum, Solitalea canadensis, Anaerostipes caccae, Eisenbergiella massiliensis, Olsenella profuse, Dorea formicigenerans, Blautia wexlerae, [Eubacterium] rectale, Pseudomonas lini, Prevotella shahii, Kroppenstedtia pulmonis, Geosporobacter ferrireducens, Mediterranea massiliensis, Staphylococcus aureus, Schaalia cardiffensis, Flavonifractor plautii, Butyricimonas virosa, Streptococcus pasteurianus, Haemophilus sputorum, and Lactobacillus mucosae is indicative of whether a cow has BRD.

In a second aspect, the present invention provides kits comprising reagents used to detect the presence or relative abundance of at least 2 biomarkers associated with bacteria. The kits are used to detect the bacteria in nasal swab samples, nasopharyngeal swab samples, or bronchoalveolar lavage samples from a cow.

For the nasal swab samples, the kits are used to detect the presence or relative abundance of at least 2 biomarkers associated with bacteria of the following species: Fusobacterium mortiferum, Prevotella stercorea, Bacteroides vulgatus, Prevotella oris, Clostridium saudiense, Lactobacillus plantarum, Bacteroides uniformis, [Clostridium] clostridioforme, Lactobacillus mucosae, Gemmiger formicilis, Prevotella copri, Terrisporobacter petrolearius, Blautia obeum, [Clostridium] scindens, Lactobacillus caviae, Ruminococcus lactaris, Catenibacterium mitsuokai, Kineothrix alysoides, Streptococcus pasteurianus, Clostridium butyricum, Lactobacillus gasseri, Holdemanella biformis, Faecalibacterium prausnitzii, Ruminococcus faecis, Fusicatenibacter saccharivorans, [Eubacterium] eligens, Butyricicoccus pullicaecorum, Blautia wexlerae, Ruminiclostridium cellobioparum, Massiliprevotella massiliensis, and Prevotellamassilia timonensis.

For the nasopharyngeal swab samples, the kits are used to detect the presence or relative abundance of at least 2 biomarkers associated with bacteria of the following species: Streptococcus uberis, Salmonella enterica, Kingella negevensis, Prevotella copri, Streptococcus pluranimalium, Holdemanella biformis, Veillonella dispar, Collinsella aerofaciens, Ruminococcus bromii, Prevotella oris, Fournierella massiliensis, Bacteroides plebeius, Lactobacillus mucosae, Alistipes finegoldii, Ruminococcus faecis, Gemmiger formicilis, Butyricicoccus pullicaecorum, Blautia wexlerae, Faecalibacterium prausnitzii, Dorea formicigenerans, Blautia obeum, Bacteroides fragilis, Coprococcus comes, Blautia luti, Dorea longicatena, [Ruminococcus] gnavus, [Eubacterium] hallii, Schaalia cardiffensis, Prevotella stercorea, and Clostridium perfringens.

For the bronchoalveolar lavage samples, the kits are used to detect the presence or relative abundance of at least 2 biomarkers associated with bacteria of the following species: Mycoplasma dispar, Mannheimia haemolytica, Moraxella caviae, Micrococcus luteus, Massilia agri, Terrimonas lutea, Alkalibacter saccharofermentans, [Clostridium] glycyrrhizinilyticum, Flavobacterium acidificum, Alistipes putredinis, Collinsella aerofaciens, Solibacillus isronensis, Monoglobus pectinilyticus, Caldalkalibacillus thermarum, Solitalea canadensis, Anaerostipes caccae, Eisenbergiella massiliensis, Olsenella profuse, Dorea formicigenerans, Blautia wexlerae, [Eubacterium] rectale, Pseudomonas lini, Prevotella shahii, Kroppenstedtia pulmonis, Geosporobacter ferrireducens, Mediterranea massiliensis, Staphylococcus aureus, Schaalia cardiffensis, Flavonifractor plautii, Butyricimonas virosa, Streptococcus pasteurianus, Haemophilus sputorum, and Lactobacillus mucosae.

### BRIEF DESCRIPTION OF THE DRAWINGS

FIG. 1 shows a plot depicting the disease status of individual calves (identified by “Animal ID” on the y-axis, “S” indicates a steer and “B” indicates a bull) after arrival to the feedlot. Calves were monitored for signs of respiratory disease every day for 30 days after arrival. Samples were taken on arrival day (Arrival, blue circle) and again on the day calves were diagnosed with BRD (BRD, red circle). When a calf was diagnosed with BRD, a sample was taken simultaneously from a healthy calf from the same pen (control, green circle). Each point represents one sample. Connected points represent samples from the same animal at two different time points.

FIG. 2A-2L show data characterizing the biogeography of the bovine respiratory microbiome. FIG. 2A shows boxplots of the alpha diversity in samples collected by nasal swab (NS), nasopharyngeal swab (NPS), and bronchoalveolar lavage (BAL) based on Shannon index. The numbers above the bars are p values calculated by the Wilcoxon test. Connected points represent samples obtained by different sampling techniques from the same animal. FIG. 2B shows a principal coordinate analysis (PCoA) plot comparing the beta diversity detected within the three niches (NS: red circles, NPS: green squares, and BAL: blue triangles) based on Jaccard distance. Each point represents one sample. FIG. 2C shows a principal coordinate analysis (PCoA) plot comparing the beta diversity detected within the three niches (NS: blue circles, NPS: green squares, and BAL: red triangles) based on Bray-curtis distance. Each point represents one sample. FIG. 2D shows a stacked bar chart comparing the average relative abundance of the top 15 operational taxonomic units (OTUs) across NS, NPS and BAL samples. FIG. 2E lists the Top 50 features identified by random forest as distinguishing samples collected by nasal swab (NS), nasopharyngeal swab (NPS), and bronchoalveolar lavage (BAL). The family and genus classifications from the Ribosomal Database Project (RDP) are provided. Features are listed based on importance score (Mean Decrease Accuracy, MDA). FIG. 2F-2H show boxplots comparing the relative abundance of OTUs that differentiate the sampling sites (NS, NPS, and BAL). The numbers above the bars are p values calculated by the Wilcoxon test. Connected points represent samples obtained by different sampling techniques from the same animal. The plots show the relative abundance of OTU13-Gammaproteobacteria (FIG. 2F), OTU1-Mycoplasma (FIG. 2G), and OTU11-Enterobacteriaceae (FIG. 2H). FIG. 2I-2L compare the relative abundance of additional OTUs across the three sampling sites (NS, NPS, and BAL). The features enriched in NS, NPS, and BAL samples are shown in FIG. 2I, FIG. 2J, FIG. 2K, and FIG. 2L, respectively. The numbers above the bars are p values calculated by the Wilcoxon test.

FIG. 3A-31I present bacterial features of the respiratory microbiome that are predictive of later onset of BRD. FIG. 3A shows the area-under-the ROC curve of the random forest model (AUC-RF) distinguishing the microbiota of healthy calves (A9) from calves diagnosed with BRD (Arrival), sampled on the day of feedlot arrival by nasal swab (NS, blue lines), nasopharyngeal swab (NPS, black lines), and bronchoalveolar lavage (BAL, red lines). “Kopt” indicates the number of features included in each model, followed by (specificity, sensitivity). FIG. 3B-3D show boxplots comparing the relative abundance of predictive OTUs in the microbiomes of healthy calves (A9) and calves diagnosed with BRD (Arrival) at feedlot arrival. The numbers above the bars are p values calculated by the Wilcoxon test. The plots show the relative abundance of OTU24-Prevotella (FIG. 3B), OTU29-Streptococcus (FIG. 3C), and OTU492-Ruminococcus (FIG. 3D). FIG. 3E lists the top 20 signatures enriched in each niche (NS, NPS, and BAL) that distinguish healthy calves from BRD calves at feedlot arrival. The family and genus classifications from the Ribosomal Database Project (RDP) are provided. Features were listed based on importance score (Mean Decrease Accuracy, MDA). The features listed in red are compared in FIG. 3D. FIG. 3F-31I show boxplots comparing the relative abundance of additional OTUs in the microbiomes of healthy (A9) and BRD calves (Arrival) at feedlot arrival. The features enriched in NS, NPS, and BAL samples are shown in FIG. 3F, FIG. 3G, and FIG. 3H, respectively. The numbers above the bars are p values calculated by the Wilcoxon test.

FIG. 4 shows principal coordinate analysis (PCoA) plots comparing the beta diversity of the microbiome at feedlot arrival (Arrival, blue circles) to that at the time of BRD diagnosis (BRD, red squares) in samples collected by nasal swab (NS), nasopharyngeal swab (NPS), and bronchoalveolar lavage (BAL) based on either Jaccard (Jaccard) or Bray-Curtis (Bray) distance. Each point represents one sample. Connected points represent samples from the same animal at two different time points, and the numbers above the lines represent the number of days between arrival and the onset of BRD.

FIG. 5A-5J depict longitudinal changes in the bovine respiratory microbiome from feedlot arrival (Arrival) to BRD onset (BRD) in samples collected by nasal swab (NS), nasopharyngeal swab (NPS), and bronchoalveolar lavage (BAL). FIG. 5A-5C list the top 20 features enriched in each niche (NS, NPS, and BAL) that are associated with onset of disease based on random forest modeling. The family or genus classifications from Ribosomal Database Project (RDP) are provided. Features were listed based on importance score (Mean Decrease Accuracy, MDA). Features listed in red are shared among the three niches. FIG. 5D-5F show boxplots comparing the relative abundance at the time of arrival to that at BRD onset for three shared OTUs (highlighted in red in FIG. 5A-5C). The plots show the relative abundance of OTU9-Mycoplasma (FIG. 5D), OTU78-Corynebacterium (FIG. 5E), and OTU207-Facklamia (FIG. 5F). FIG. 5G shows the area-under-the ROC curve of the random forest model (AUC-RF) distinguishing the microbiota of calves at feedlot arrival (Arrival) to that at BRD onset (BRD) based on samples obtained by nasal swab (NS, blue lines), nasopharyngeal swab (NPS, black lines), and bronchoalveolar lavage (BAL, red lines). “Kopt” indicates the number of features included in each model, followed by (specificity, sensitivity). FIG. 511-5J show boxplots comparing the relative abundance of additional features at the time of feedlot arrival (Arrival) to that at the time of BRD onset (BRD). The signatures enriched in NS, NPS, and BAL samples are shown in FIG. 5H, FIG. 5I, and FIG. 5J, respectively. The numbers above the bars are p values calculated by the Wilcoxon test.

FIG. 6 shows principal coordinate analysis (PCoA) plots comparing the beta diversity of the microbiome of healthy control calves (control, green triangles) to that of calves diagnosed with BRD (BRD, red squares) in samples collected by nasal swab (NS), nasopharyngeal swab (NPS), and bronchoalveolar lavage (BAL) based on either Jaccard (Jaccard) or Bray-Curtis (Bray) distance. Each point represents one sample. Points representing samples from BRD calves are connected to the points representing samples from their paired control.

FIG. 7 lists the top 20 features identified by area-under-the ROC curve of the random forest model (AUC-RF) as differentiating healthy control calves (control) from calves diagnosed with BRD (BRD) based on samples collected by nasal swab (NS), nasopharyngeal swab (NPS), and bronchoalveolar lavage (BAL). The family and genus classifications from the Ribosomal Database Project (RDP) are provided. Features were listed based on importance score (Mean Decrease Accuracy, MDA). The relative abundance of features listed in red are shown in FIG. 8B-8D.

FIG. 8A-8G present bacterial features of the respiratory microbiome that distinguish healthy calves from calves with BRD. FIG. 8A shows an area-under-the ROC curve of the random forest model (AUC-RF) comparing the microbiota of healthy control calves (control) to that of calves diagnosed with BRD (BRD) based on samples obtained by nasal swab (NS, blue lines), nasopharyngeal swab (NPS, black lines), and bronchoalveolar lavage (BAL, red lines). “Kopt” indicates the number of features included in each model, followed by (specificity, sensitivity). FIG. 8B-8D show boxplots comparing the relative abundance of OTUs in healthy control calves (control) to the abundance in calves diagnosed with BRD (BRD). The plots show the relative abundance of OTU144-Lactobacillus (FIG. 8B), OTU45-Clostridium_sensu_stricto (FIG. 8C), and OTU76-Clostridium_sensu_stricto (FIG. 8D). FIG. 8E-8G show boxplots comparing the relative abundance of additional OTUs in healthy control calves (control) to the abundance in calves diagnosed with BRD (BRD). The features enriched in NS, NPS, and BAL samples are shown in FIG. 8E, FIG. 8F, and FIG. 8G, respectively. The numbers above the bars are p values calculated by the Wilcoxon test.

### DETAILED DESCRIPTION

The present invention provides methods and kits for selecting cows to treat for bovine respiratory disease (BRD) based on the levels of biomarkers in the respiratory microbiome of the cows. In the Examples, the applicants disclose sets of bacterial operational taxonomic units (OTUs) that were identified from bovine nostrils, nasopharynx, and lungs, which can be used as biomarkers to (1) predict the likelihood that a calf will develop BRD or (2) diagnose a calf with BRD. The ability to selectively treat only calves deemed to be at risk for BRD would greatly benefit producers in various cattle industries. With this ability, producers can omit calves that are classified as “low risk” when applying antibiotic therapies to the herd, saving them money and decreasing antibiotic use. Further, calves that are deemed “high risk” can be treated more intensively at an earlier stage, ultimately reducing the costs of medication and the losses in growth performance related to BRD.

As is alluded to above, two sets of biomarkers that are useful for selecting cows to treat for BRD are disclosed in the present application. The first set includes biomarkers that can be used to predict whether a calf is likely to develop BRD. As is detailed in the Examples, respiratory microbiome samples were taken from calves upon arrival to a feedlot. After the health outcome of each calf was determined, this set of predictive biomarkers was identified by comparing the microbes present in calves that became sick to those present in calves that remained healthy. The second set of biomarkers distinguish calves that currently have BRD from healthy calves. These diagnostic biomarkers were identified by comparing the microbes present in calves that had just been diagnosed with BRD to those present in healthy calves from the same pen. While these two sets of biomarkers are largely distinct, there is some overlap between them, both at the level of bacterial species and at the level of specific OTUs. The greatest overlap exists in the sets of biomarkers identified from the nasal microbiome, which contain six of the same OTUs. Given that a calf should be treated for BRD whether it is likely to develop BRD or has already developed BRD, the presence or levels of a biomarker from either set can be used to select calves for treatment. However, under certain circumstances it may be useful to tailor the treatment based on the more precise prognosis granted by separate use of these two sets.

In the methods of the present invention, the abundance of one or more biomarkers is analyzed to determine whether to treat the cow. In some embodiments, the analysis of particular biomarkers will be qualitative, i.e., based simply on whether the biomarker is present in the sample at detectable levels or not. Other biomarkers will be analyzed quantitatively, by comparing the levels of the biomarker in a tested sample to levels of the biomarker in a control sample. A “control sample”, as used herein, is a sample taken from a healthy cow (i.e., a cow without any detectable symptoms of BRD and suitably a cow that will not get BRD). Ideally, the control sample is of the same sample type (i.e., NS, NPS, or BAL) as the sample being tested and is representative of the mean level of the biomarkers found across healthy cows. However, in some cases, it may not be feasible to determine the mean level of the biomarker due to the wide variation that exists across the microbiomes of healthy individuals. In such cases, the mean level found in the cohort of cows being brought to the feed lot at the same time may also be used as a control.

In some embodiments, a sample of the respiratory microbiome is obtained by nasal swab (NS), while in other embodiments a sample is obtained by nasopharyngeal swab (NPS) or by bronchoalveolar lavage (BAL). Each of these sampling methods assays a different niche of the respiratory microbiome, the nasal cavity, nasopharynx, and lungs, respectively. Details of these sampling methods are provided in the Examples. Those of skill in the art are familiar with the methods for collection, maintenance, and preparation of such samples.

BRD is a particularly costly problem for the beef industry. However, the methods of the present invention may be utilized by producers in any cattle industry, including those that use cattle for the production of beef, hides, dairy, and other products.

The physical and psychological stress associated with weaning calves and transporting them to a new location increases their susceptibility to infections such as BRD. Thus, while the methods of the present invention may be applied to a cow at any developmental stage and at any geographical location, in preferred embodiments, the risk of BRD is assessed after a calf has been weaned and/or transported to a new location, such as a feedlot.

Methods:

In a first aspect, the present invention provides methods for selecting cows to treat for bovine respiratory disease (BRD). The methods include collecting a nasal swab, nasopharyngeal swab, or bronchoalveolar lavage sample from a cow, measuring the level of at least one biomarker associated with a bacterium, and analyzing the abundance of the biomarker to determine whether to treat the cow.

In embodiments utilizing a nasal swab sample, the level of a biomarker associated with a bacterium of a species from the group consisting of: Fusobacterium mortiferum, Prevotella stercorea, Bacteroides vulgatus, Prevotella oris, Clostridium saudiense, Lactobacillus plantarum, Bacteroides uniformis, [Clostridium] clostridioforme, Lactobacillus mucosae, Gemmiger formicilis, Prevotella copri, Terrisporobacter petrolearius, Blautia obeum, [Clostridium] scindens, Lactobacillus caviae, Ruminococcus lactaris, Catenibacterium mitsuokai, Kineothrix alysoides, and Streptococcus pasteurianus is indicative of the likelihood that a cow will develop BRD, while the level of a biomarker associated with a bacterium of a species selected from Clostridium butyricum, Lactobacillus gasseri, Holdemanella biformis, Clostridium saudiense, Catenibacterium mitsuokai, Faecalibacterium prausnitzii, Prevotella stercorea, Ruminococcus faecis, Prevotella copri, Fusicatenibacter saccharivorans, Gemmiger formicilis, [Eubacterium] eligens, Butyricicoccus pullicaecorum, Blautia wexlerae, Ruminiclostridium cellobioparum, Massihprevotella massiliensis, Prevotellamassilia timonensis, and Lactobacillus mucosae is indicative of whether a cow has BRD. Here, the cow will be treated for BRD if one or more of the following differences in the abundance of a bacterial species is detected: a decrease in Fusobacterium mortiferum, decrease in Prevotella stercorea, decrease in Bacteroides vulgatus, decrease in Prevotella oris, decrease or increase in Clostridium saudiense (wherein a decrease indicates that the cow is likely to get BRD and an increase indicates that the cow has BRD), increase in Lactobacillus plantarum, decrease in Bacteroides uniformis, decrease in [Clostridium] clostridioforme, decrease or increase in Lactobacillus mucosae (wherein a decrease indicates that the cow has BRD and an increase indicates that the cow is likely to get BRD), decrease in Gemmiger formicilis, decrease in Prevotella copri, decrease in Terrisporobacter petrolearius, increase in Blautia obeum, decrease in [Clostridium] scindens, increase in Lactobacillus caviae, increase in Ruminococcus lactaris, decrease or increase in Catenibacterium mitsuokai (wherein a decrease indicates that the cow has BRD and an increase indicates that the cow is likely to get BRD), increase in Kineothrix alysoides, increase in Streptococcus pasteurianus, increase in Clostridium butyricum, decrease in Lactobacillus gasseri, decrease in Holdemanella biformis, decrease in Faecalibacterium prausnitzii, decrease in Ruminococcus faecis, decrease in Fusicatenibacter saccharivorans, decrease in [Eubacterium] eligens, decrease in Butyricicoccus pullicaecorum, decrease in Blautia wexlerae, increase in Ruminiclostridium cellobioparum, decrease in Massiliprevotella massiliensis, or decrease in Prevotellamassilia timonensis. If the presence of a biomarker associated with Lactobacillus plantarum is detected, then the cow should be treated.

In certain embodiments, the biomarkers measured in the nasal microbiome are associated with bacteria that belong to one or more of the following strains: Fusobacterium mortiferum strain DSM 19809, Prevotella stercorea DSM 18206 strain CB35, Bacteroides vulgatus ATCC 8482, Prevotella oris strain JCM 12252, Clostridium saudiense strain JCC, Lactobacillus plantarum strain CIP 103151, Bacteroides uniformis strain JCM 5828, [Clostridium] clostridioforme strain ATCC 25537, Lactobacillus mucosae strain S32, Gemmiger formicilis strain X2-56, Prevotella copri DSM 18205 strain JCM 13464, Terrisporobacter petrolearius strain LAMOA37, Blautia obeum ATCC 29174, [Clostridium] scindens strain ATCC 35704, Lactobacillus caviae strain MOZM2, Ruminococcus lactaris ATCC 29176, Catenibacterium mitsuokai strain DSM 15897, Kineothrix alysoides strain KNHs209, Streptococcus pasteurianus strain CIP 107122, Clostridium butyricum strain JCM 1391, Lactobacillus gasseri ATCC 33323=JCM 1131, Holdemanella biformis strain DSM 3989, Faecalibacterium prausnitzii strain ATCC 27768, Ruminococcus faecis JCM 15917 strain Eg2, Fusicatenibacter saccharivorans strain HT03-11, [Eubacterium] eligens ATCC 27750, Butyricicoccus pullicaecorum strain 25-3, Blautia wexlerae DSM 19850, Ruminiclostridium cellobioparum DSM 1351=ATCC 15832 strain JCM 1422, Massiliprevotella massiliensis strain Marseille-P2439, and Prevotellamassilia timonensis strain Marseille-P2831.

In embodiments utilizing nasopharyngeal swab samples, the level of a biomarker associated with a bacterium of a species from the group consisting of: Streptococcus uberis, Salmonella enterica, Kingella negevensis, Prevotella copri, Streptococcus pluranimalium, Holdemanella biformis, Veillonella dispar, Collinsella aerofaciens, Ruminococcus bromii, Prevotella oris, Fournierella massiliensis, Bacteroides plebeius, Lactobacillus mucosae, and Alistipes finegoldii is indicative of the likelihood that a cow will develop BRD, while the level of a biomarker associated with a bacterium of a species selected from Ruminococcus faecis, Prevotella copri, Gemmiger formicilis, Butyricicoccus pullicaecorum, Blautia wexlerae, Faecalibacterium prausnitzii, Dorea formicigenerans, Blautia obeum, Bacteroides Coprococcus comes, Blautia luti, Dorea longicatena, [Ruminococcus] gnavus, [Eubacterium] hallii, Schaalia cardiffensis, Prevotella stercorea, and Clostridium perfringens is indicative of whether a cow has BRD. Here, the cow will be treated for BRD if one or more of the following differences in the abundance of a bacterial species is detected: an increase in Streptococcus uberis, increase in Salmonella enterica, decrease in Kingella negevensis, decrease or increase in Prevotella copri depending on the 16S rRNA sequence (wherein a decrease in OTU24 (SEQ ID NO:11) indicates that the cow is likely to get BRD and an increase indicates that the cow has BRD), increase in Streptococcus pluranimalium, decrease in Holdemanella biformis, decrease in Veillonella dispar, decrease in Collinsella aerofaciens, decrease in Ruminococcus bromii, decrease in Prevotella oris, decrease in Fournierella massiliensis, decrease in Bacteroides plebeius, decrease in Lactobacillus mucosae, decrease in Alistipes finegoldii, increase in Ruminococcus faecis, increase in Gemmiger formicilis, increase in Butyricicoccus pullicaecorum, increase in Blautia wexlerae, increase in Faecalibacterium prausnitzii, increase in Dorea formicigenerans, increase in Blautia obeum, decrease in Bacteroides fragilis, increase in Coprococcus comes, increase in Blautia luti, increase in Dorea longicatena, decrease or increase in [Ruminococcus] gnavus depending on the 16S rRNA sequence, increase in [Eubacterium] hallii, decrease in Schaalia cardiffensis, increase in Prevotella stercorea, or decrease in Clostridium perfringens. If the presence of a biomarker associated with Kingella or Alistipes is not detected, then the cow should be treated. If the presence of the biomarker OTU365 (SEQ ID NO:69) or OTU24 (SEQ ID NO:11) or a biomarker associated with the bacterial species Gemmiger formicilis, Dorea formicigenerans, Dorea longicatena, Ruminococcus faecis, Blautia obeum, Blautia luti, or Prevotella stercorea is detected, then the cow should be treated.

In certain embodiments, the biomarkers measured in the nasopharyngeal microbiome are associated with bacteria that belong to one or more of the following strains: Streptococcus uberis strain JCM 5709, Salmonella enterica subspecies enterica serovar Typhimurium strain ATCC 13311, Kingella negevensis strain Sch538, Prevotella copri DSM 18205 strain JCM 13464, Streptococcus pluranimalium strain T70, Holdemanella biformis strain DSM 3989, Veillonella dispar strain ATCC 17748, Collinsella aerofaciens strain JCM 10188, Ruminococcus bromii strain ATCC 27255, Prevotella oris strain JCM 12252, Fournierella massiliensis strain AT2, Bacteroides plebeius DSM 17135 strain M12, Lactobacillus mucosae strain S32, Alistipes finegoldii strain DSM 17242, Ruminococcus faecis JCM 15917 strain Eg2, Gemmiger formicilis strain X2-56, Butyricicoccus pullicaecorum strain 25-3, Blautia wexlerae DSM 19850, Faecalibacterium prausnitzii strain ATCC 27768, Dorea formicigenerans strain ATCC 27755, Blautia obeum ATCC 29174, Bacteroides fragilis strain NCTC 9343, Coprococcus comes ATCC 27758, Blautia luti strain BInIX, Dorea longicatena strain 111-35, [Ruminococcus] gnavus ATCC 29149, [Eubacterium] hallii strain ATCC 27751, Schaalia cardiffensis strain CCUG 44997, Prevotella stercorea DSM 18206 strain CB35, and Clostridium perfringens ATCC 13124.

In embodiments utilizing bronchoalveolar lavage samples, the level of a biomarker associated with a bacterium of a species from the group consisting of: Mycoplasma dispar, Mannheimia haemolytica, Moraxella caviae, Micrococcus luteus, Massilia agri, Terrimonas lutea, Alkalibacter saccharofermentans, [Clostridium] glycyrrhizinilyticum, Flavobacterium acidificum, Alistipes putredinis, Collinsella aerofaciens, Solibacillus isronensis, and Monoglobus pectinilyticus is indicative of the likelihood that a cow will develop BRD, while the level of a biomarker associated with a bacterium of a species selected from Caldalkalibacillus thermarum, Solitalea 13 anadensis, Anaerostipes caccae, Eisenbergiella massiliensis, Olsenella profuse, Dorea formicigenerans, Blautia wexlerae, [Eubacterium] rectale, Pseudomonas lini, Prevotella shahii, Kroppenstedtia pulmonis, Geosporobacter ferrireducens, Mediterranea massiliensis, Staphylococcus aureus, Schaalia cardiffensis, Flavonifractor plautii, Butyricimonas virosa, Streptococcus pasteurianus, Haemophilus sputorum, and Lactobacillus mucosae is indicative of whether a cow has BRD. Here, the cow will be treated for BRD if one or more of the following differences in the abundance of a bacterial species is detected: a decrease in Mycoplasma dispar, decrease in Mannheimia haemolytica, decrease in Moraxella caviae, decrease in Micrococcus luteus, decrease in Massilia agri, decrease in Terrimonas lutea, increase in Alkalibacter saccharofermentans, increase in [Clostridium] glycyrrhizinilyticum, decrease in Flavobacterium acidificum, decrease in Alistipes putredinis, increase in Collinsella aerofaciens, decrease in Solibacillus isronensis, decrease in Monoglobus pectinilyticus, increase in Caldalkalibacillus thermarum, increase in Solitalea canadensis, increase in Anaerostipes caccae, decrease in Eisenbergiella massiliensis, decrease in Olsenella profuse, increase in Dorea formicigenerans, increase in Blautia wexlerae, decrease in [Eubacterium] rectale, decrease in Pseudomonas lini, decrease in Prevotella shahii, increase in Kroppenstedtia pulmonis, decrease in Geosporobacter ferrireducens, increase in Mediterranea massiliensis, increase in Staphylococcus aureus, decrease in Schaalia cardiffensis, increase in Flavonifractor plautii, decrease in Butyricimonas virosa, decrease in Streptococcus pasteurianus, decrease in Haemophilus sputorum, and decrease in Lactobacillus mucosae. If the presence of a biomarker associated with Moraxella is not detected, then the cow is treated.

In certain embodiments, the biomarkers measured in the lung microbiome are associated with bacteria that belong to one or more of the following strains: Mycoplasma dispar strain 462/2, Mannheimia haemolytica strain NCTC 9380, Moraxella caviae strain GP11, Micrococcus luteus strain NCTC 2665, Massilia agri strain K-3-1, Terrimonas lutea strain DY, Alkalibacter saccharofermentans strain Z-79820, [Clostridium] glycyrrhizinilyticum strain ZM35, Flavobacterium acidificum strain LMG 8364, Alistipes putredinis strain JCM 16772, Collinsella aerofaciens strain JCM 10188, Solibacillus isronensis B3W22, Monoglobus pectinilyticus strain 14, Caldalkalibacillus thermarum strain HA6, Solitalea canadensis DSM 3403, Anaerostipes caccae strain L1-92, Eisenbergiella massiliensis strain AT11, Olsenella profusa DSM 13989, Dorea formicigenerans strain ATCC 27755, Blautia wexlerae DSM 19850, [Eubacterium] rectale ATCC 33656, Pseudomonas lini strain DLE411J, Prevotella shahii strain EHS11, Kroppenstedtia pulmonis strain W9323, Geosporobacter ferrireducens strain IRF9, Mediterranea massiliensis strain Marseille-P2645, Staphylococcus aureus strain S33 R, Schaalia cardiffensis strain CCUG 44997, Flavonifractor plautii strain Prevot S1, Butyricimonas virosa strain MT12, Streptococcus pasteurianus strain CIP 107122, Haemophilus sputorum CCUG 13788, and Lactobacillus mucosae strain S32.

The respiratory microbiome bacteria described above and in the Examples were classified based on current classifications of bacteria from the Ribosomal Database Project [17] using ribosomal RNA gene sequencing data. Those of skill in the art will appreciate that the names and strain designations of bacteria sometimes change over time as more information becomes available. Thus, the present application also provides the specific ribosomal sequences (listed in Table 2-7) that were detected in the samples in addition to the names of the bacterial strains that were associated with these sequences at the time these experiments were completed. Table 2, Table 3, and Table 4 list the partial 16S rRNA sequences that can be used to predict BRD in samples collected by nasal swab (NS), nasopharyngeal swab (NPS), and bronchoalveolar lavage (BAL), respectively. Table 5, Table 6, and Table 7 list the partial 16S rRNA sequences that can be used to diagnose BRD in samples collected by nasal swab (NS), nasopharyngeal swab (NPS), and bronchoalveolar lavage (BAL), respectively.

As used herein, the term “biomarker” refers to a molecule that is differentially expressed in a particular condition. The biomarkers of the present invention are related to bacteria that are differentially expressed in (1) cows that ultimately developed BRD as compared to cows that remained healthy, and (2) cows that currently have BRD as compared to healthy cows (i.e., cows without any detectable symptoms of BRD). The biomarkers utilized in the present invention may include any protein or nucleic acid that is specific to a bacterium described herein, such that detection of the biomarker in a sample is indicative of the presence of that bacterium in the sample.

In some embodiments, the biomarkers are proteins that are associated with particular bacteria. In the present application, the terms “polypeptide”, “protein”, and “peptide” are used interchangeably herein to refer to a series of amino acid residues connected to by peptide bonds between the alpha-amino and carboxy groups of adjacent residues, forming a polymer of amino acids.

Detection of proteins may be performed using antibodies that specifically recognize the bacterial proteins. The term “specific” refers to the ability of a protein to bind one molecule in preference to other molecules. An antibody that is specific to a target protein binds to the target protein but does not bind in a significant amount to other molecules present in the sample. Specific binding can mean binding to a target with an affinity that is at least 25% greater, at least 50% greater, at least 100% (2-fold) greater, at least ten times greater, at least 20-times greater, or at least 100-times greater than the affinity to any other molecule.

Antibody-antigen recognition may be analyzed using a variety of methods known to those of skill in the art including, but not limited to, ELISA (enzyme-linked immunosorbent assay), western blotting, dot blotting, immunohistochemistry, immunocytochemistry, fluorescence-activated cell sorting (FACS), immunoprecipitation, fluorescence microscopy, and protein microarray.

In other embodiments, the biomarkers are nucleic acids that are associated with particular bacteria. In the present application, the terms “nucleic acid”, “polynucleotide”, and “oligonucleotide” are used interchangeably to refer to molecules of DNA and/or RNA.

Nucleic acids can be “isolated” or “extracted” from a biological sample for analysis using standard techniques known in the art including those that rely on organic extraction, ethanol precipitation, silica-binding chemistry, cellulose-binding chemistry, and ion exchange chemistry. Many reagents and kits for performing nucleic acid extractions are commercially available.

Detection of nucleic acids may be performed using one or more oligonucleotide probes or primers that selectively hybridize to a target nucleic acid that includes one or more of the biomarkers through complementary base pairing. As is known to those of skill in the art, a probe or primer does not need to be perfectly complementary to a target sequence in order to hybridize with it, and it can be modified in a number of ways (e.g., methylation, fluorescent tagging) without altering its basic function.

In some embodiments, primers are used to detect the presence of nucleic acid biomarkers by amplification. In these embodiments, amplification of a product indicates the presence of the biomarker in the sample. Amplification-based methods include polymerase chain reaction (PCR) and primer extension reactions. Suitable PCR-based methods include, without limitation, standard PCR, quantitative PCR (qPCR), PCR-restriction fragment length polymorphism (PCR-RFLP), asymmetrical PCR, strand displacement amplification (SDA), rolling circle amplification (RCA), transcript mediated amplification (TMA), self-sustained sequence replication (3 SR), and ligase chain reaction (LCA). The amplification product can be detected directly or indirectly by any method known in the art, including, but not limited to, visualization with ethidium bromide, label incorporation, and dye intercalation. The amplification product may also be sequenced using methods known to those skilled in the art.

Other known hybridization-based methods of detection may also be utilized in the present invention. These methods generally rely on the detection of labeled probes (e.g., radioactively, fluorescently, and chemiluminescently labeled probes) that anneal to the target nucleic acid. Common hybridization-based methods include in situ hybridization, microarray analysis, oligonucleotide ligation assays, and Southern or northern blotting. In these methods, detection may involve comparing the amount of labeled probe that binds to target nucleic acid molecule as compared to a nucleic acid molecule other than the target molecule, particularly a substantially similar (i.e., homologous) nucleic acid molecule. Conditions that allow for selective hybridization can be determined empirically, or can be estimated based, for example, on the relative GC:AT content of the probe and the sequence to which it hybridizes, the length of the probe, or the number of mismatches between the probe and sequence to which it is to hybridize.

Many additional methods for detecting nucleic acids are known in the art and are encompassed by the present invention. These methods include those that rely on differential endonuclease digestion, such as restriction fragment length polymorphism (RFLP) analysis. Sequencing methods, mass spectrometry, scanning electron microscopy, or methods in which a polynucleotide flows past a sorting device that can detect the sequence of the polynucleotide may also be utilized. For instance, in the Examples of the present invention, the biomarkers are detected using high-throughput sequencing followed by data analysis. Useful methods include those that are readily adaptable to a high throughput format, to a multiplex format, or to both.

In certain embodiments of the invention, the biomarkers are measured quantitatively, to determine the abundance of the biomarkers in the microbiome sample relative to the abundance in a control sample. Quantitative methods of nucleic acid detection include, without limitation, arrays (e.g., microarrays), high-throughput sequencing, and real time PCR.

In some embodiments, the nucleic acid biomarkers are components of a ribosomal subunit. The sequences of ribosomal RNA (rRNA) genes, including 16S rRNA and 23S rRNA, are commonly used to identify and compare the bacteria or fungi present within a sample since they are found across nearly all forms of life. In certain embodiments, the nucleic acids comprise V4 regions of 16S rRNA genes listed in Table 2-7 and utilized in the Examples.

The microbiome samples may be analyzed by individuals practicing the methods of the present invention, or alternatively, they may be analyzed by a separate entity, such as an independent testing laboratory.

In some embodiments, the methods further comprise treating the selected cows for BRD. Any method of treating BRD may be used with the present invention. Standard treatments for BRD include vaccines against viruses that initiate the disease and antimicrobial treatments (e.g., broad-spectrum antibiotics) that work against bacterial forms of BRD. In addition treatment may include nonsteroidal anti-inflammatories (NSAIDS) or other immunomodulators. Vaccines may be targeted to those animals identified as at risk of BRD.

Kits:

In a second aspect, the present invention provides kits comprising reagents that may be used to detect the presence of the biomarkers described herein. In some embodiments, the kits are designed to detect the presence of biomarkers in nasal swab samples. In other embodiments, the kits are designed to detect the presence of biomarkers in nasopharyngeal swab samples. In still other embodiments, the kits are designed to detect the presence of biomarkers in bronchoalveolar lavage samples. In certain embodiments, the presence of particular biomarkers is assessed qualitatively, while in other embodiments, the biomarkers are assessed quantitatively.

The kits of the present invention may utilize any known method for detecting proteins or nucleic acids, including the methods of detection described above. In some embodiments, the kits of the present invention comprise antibodies specific to proteins associated with particular bacteria. The term “antibody” refers to immunoglobulin molecules, or other molecules that comprise an antigen-binding domain from an immunoglobulin molecule, that recognize and specifically bind to a target molecule. Suitable antibodies include, without limitation, whole antibodies (e.g., IgG, IgA, IgE, IgM, or IgD), monoclonal antibodies, polyclonal antibodies, chimeric antibodies, humanized antibodies, and antibody fragments, including single chain variable fragments (ScFv), single domain antibodies, antigen-binding fragments (e.g., complementarity determining region (CDR) domains), and genetically engineered antibodies. Thus, any form of antibody, antibody fragment, or antibody-derived fragment may be used with the present invention.

In other embodiments, the kits comprise sets of PCR primers that amplify nucleic acids associated with particular bacteria. As used herein, the term “primer” refers to a single-stranded nucleic that is used to initiate DNA synthesis. The term “PCR primer” refers to a primer used in a PCR reaction. In certain preferred embodiments, the kits use PCR primers to amplify nucleic acids that are components of the 16S or 23S ribosomal subunits of specific bacteria.

The kits may contain additional reagents for performing methods described herein including, but not limited to, one or more detectable labels, which can be used to label a probe or primer or can be incorporated into a product generated using primer (e.g., an amplification product); one or more polymerases, which can be useful for a method that includes a primer extension or amplification procedure; or other enzymes (e.g., a ligase or an endonuclease), which can be useful for performing an oligonucleotide ligation assay or a mismatch endonuclease cleavage assay; and/or one or more buffers or other reagents that are necessary to or can facilitate performing the methods. The kits may also include instructions for performing the method or for analyzing the results and making predictions based on the results.

In some embodiments, the kits comprise one or more control samples. Suitable control samples include samples from healthy cows (i.e., cows without any detectable symptoms of BRD) and samples from cows with BRD, to be used as negative and positive controls, respectively. The controls may also be simple positive and negative controls artificially generated to ensure the methods are working properly.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The following examples are meant only to be illustrative and are not meant as limitations on the scope of the invention or of the appended claims.

### EXAMPLES

The respiratory microbiome plays an essential role in the pathophysiology of bovine respiratory disease (BRD). A better understanding of this role will facilitate the development of alternative therapies or management strategies for the prevention of BRD. Several previous studies have explored the nasopharyngeal microbiota and their relationship with BRD [6-10]. In these studies, significant changes were observed in the nasopharyngeal microbiota of calves during their first 60 days at feedlot [7, 11]. In calves diagnosed with BRD, a significant reduction in bacterial diversity was observed in the nasopharynx, both upon feedlot entry and 60 days after placement [3, 12], suggesting that the nasopharyngeal microbiota present during feedlot entry may affect the pathophysiology of BRD [13, 14].

While these studies have expanded our understanding of the microbiome of the bovine airway, little is known about the other microbial niches within the bovine respiratory system [1, 14]. Thus, comprehensive studies characterizing the biogeography of the bovine respiratory microbiome are lacking and greatly needed. In the following Example, the inventors characterize the microbiome in three niches of the respiratory system: the nasal cavity, nasopharynx, and lung. Further, they examine how these communities change leading up to the onset of BRD.

Materials and Methods:

Experimental design: This study was designed to include both a longitudinal and a cross-sectional analysis. The weaned calves were monitored for symptoms of BRD each day after they arrived in the feedlot to produce a longitudinal comparison, and healthy calves were utilized as controls in a cross-sectional comparison.

Animals: Forty-eight newly weaned Angus beef calves were used in this study. All calves were initially healthy, with no history of receiving antimicrobial drugs prior to or after arrival at the feedlot. On arrival to the feedlot (d0), all calves were given access to hay and water and were rested overnight in holding pens. On the following morning (dl), calves were stratified by weight and gender (high risk calves could be intact or castrated) and were allocated randomly to different pens, with 12 calves per pen. On the same day (dl), calves were vaccinated against clostridial toxins and bovine respiratory viruses using a commercially available modified-live multivalent vaccine, were given an anthelmintic, and were castrated (if necessary). Calves were fed a standard diet that meets their nutritional requirements.

Microbiome sample collection: All calves were sampled using nasal swabs (NS), nasopharyngeal swabs (NPS), and bronchoalveolar lavage (BAL). NS were collected by swirling two Puritan Opti-Swabs (Puritan Medical Products Co. LLC, Guilford, Me.) in the mid-nare region of the nose until they were saturated. NPS were collected by inserting a double guarded culture swab (Jorgensen Labs, Loveland, Colo.) up the nares until reaching the nasopharynx where the swab was advanced through the guard, rotated against the nasopharyngeal mucosa, and then retracted back into the guard and removed from the nares. In BAL sampling, fluid is squirted into a small part of the lung and then collected for examination. This method samples the lower generation bronchi and alveolar spaces. To retrieve a BAL sample, a bal-240 tube (MILA International, Florence, Ky.) was passed through the nares, guided through the larynx into the trachea, and advanced until resistance was met. Sterile 0.9% saline was administered in aliquots of 60 ml (up to 240 ml) and aspirated.

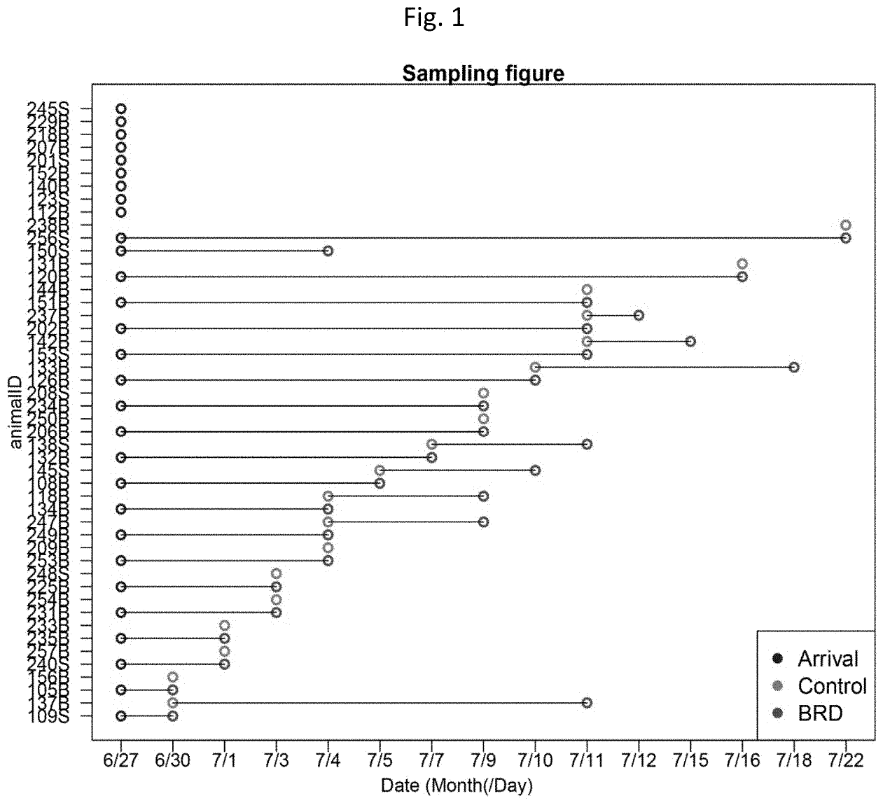

Respiratory health assessment: Calves were monitored for clinical respiratory disease daily for 30 days following feedlot arrival. Microbiome samples were taken upon arrival to the feedlot (Arrival) and then on the day calves were diagnosed with BRD. Nine calves remained healthy throughout the course of this study (referred to as A9). Following diagnosis with BRD, samples were taken simultaneously from the sick calve and from a healthy calf from the same pen (FIG. 1). Continued monitoring of the healthy control calves revealed that eight of these calves later developed BRD. However, the healthy control calves never got BRD are CNB (Control no BRD), the calves at the day sampling as control and diagnosed BRD later were CPB (control prior to BRD), and the day they got BRD was CSB (Control diagnosed with BRD).

Diagnoses of BRD were made by trained feedlot personnel. If any animal exhibited at least two symptoms consistent with BRD (depression, nasal discharge, ocular discharge, cough, gaunt appearance, or inappetence) it was moved to a hospital facility and its rectal temperature was recorded. Based on rectal temperature, a calf was placed in one of two BRD diagnostic categories: 1) if the rectal temperature was >40° C., the animal was diagnosed with “febrile” BRD; and 2) if the rectal temperature was <40° C., the animal was diagnosed with “non-febrile” BRD. Regardless of its designation, each BRD calf received the same antimicrobial treatment regimen. After BRD diagnoses, the calves were administered ceftiofur crystalline free acid (Excede, Zoetis, Kalamazoo, Mich.) at 6.6 mg/kg bodyweight. If a calf was diagnosed with BRD again, a second antimicrobial regimen was administered, which consisted of florfenicol (Nuflor, Merck Animal Health, Summit, N.J.) at 40 mg/kg bodyweight. Upon a third BRD diagnosis, calves were treated with a final antimicrobial regimen, which consisted of oxytetracycline (4.4 mg/kg bodyweight; Norbrook Inc., Lenexa, Kans.).

DNA Extraction and next-generation sequencing: DNA was extracted using the DNeasy PowerLyzer PowerSoil Kit (Qiagen, Germantown, Md.). Sterile Opti-Swab Amies buffer was taken through the extraction process to serve as a negative control. DNA standards (ZymoBIOMICS Microbial Community) were included as a positive control. The V4 region of the 16S rDNA gene was amplified and sequenced on an Illumina MiSeq 2×150 bp platform. From each sample, a 10 ng/μL DNA aliquot was used to construct a sequencing library targeting the V4 region of 16S rRNA. All samples were amplified with dual-index primers via PCR and amplicons were normalized using a SequalPrep™ Normalization kit (Life Technologies Corp., Grand Island, N.Y.). PCR amplicons from each sample possessed specific barcode sequences to differentiate them within the pooled library. A 5 μL aliquot of each normalized sample was combined to generate a pooled library. Both library concentration and exact product size were measured using a KAPA Library Quantification Kit (Kapa Biosystems, Woburn, Mass.) utilizing a quantitative PCR Eppendorf Realplex4 (Eppendorf, Hamburg, Germany) assay and an Agilent 2100 Bioanalyzer System (Agilent, Santa Clara, Calif.), respectively. Based on the qPCR and Bioanalyzer results, the pooled library was diluted to 2 nM prior to sequencing. Next-generation sequencing was performed on an Illumina MiSeq sequencer (Illumina, San Diego, Calif.).

Bioinformatics and statistics: The software package mothur v. 1.39.1 [15] was used to analyze the next-generation sequencing data. Briefly, contigs between read pairs were assembled. Sequencing errors were reduced using a pre-clustering algorithm [16]. The sequences were aligned with the SILVA reference database (full-length sequences and taxonomy references release 128, www.arb-silva.de/). Chimeras were removed using the VSEARCH algorithm. High quality sequences were binned into operational taxonomic units (OTUs) at the 97% similarity level and were classified using the Ribosomal Database Project [17]. Sequences were randomly subsampled to the smallest number of reads to minimize the effect of sequencing depth on alpha and beta diversity measures. Bray-Curtis and Jaccard distance metrics were calculated to investigate differences in community structure. ANalysis Of SIMilarity was employed to compare the significance of beta diversity. A principal coordinate analysis (PCoA) plot was made based on the measured distances in R (v. 3.5.3).

Random forest was used to identify the top bacterial features that are most predictive of BRD. A plot of variable importance was generated by ranking the features by their importance scores (Mean Decrease Accuracy). In this study, the top 50 features with a mean decrease accuracy above 3 were considered important predictors. The R package ‘RandomForest v. 4.6-7’ was used to perform random forest processing. The ‘importance’ and ‘proximity’ parameters were set as ‘True’ and ‘ntree’ was set to 10000 in the model. The alpha diversity (Shannon Index, chao and observed OTUs) and the top 500 OTUs were used to classify the predictors using the AUCRF R package (v. 1.1). A leave-one-subject out method was used in the AUCRF model, and a 10-fold cross-validation (AUCRFcv) was set to estimate the prediction error of the model. Thus, the model predicted the left-out subject and results were plotted as Receiver Operator Characteristic curves using the pROC package (v. 1.13). The optimal predictors of AUCRF were listed based on their mean decrease accuracy (MDA). Boxplots of relative abundance of optimal predictors were created using the R ggplot2 package (v. 3.0) and p values were calculated from a Wilcoxon test.

To estimate microbial drift from the upper airway to the lung, the neutral model was performed based on methods described by Pragman et al. (2018) [18]. In this model, a species with high abundance in the source environment would have a greater chance of detection in the lungs due to continued dispersal. The relative abundance of OTUs in source sites and the frequency of each OTU in the lungs were calculated. Then, a beta distribution was applied to estimate neutral movement of microbes. OTUs that fell within the 95% confidence intervals were deemed to fit the neutral model curve. The taxon that fell above the upper bound of the confidence intervals were deemed over-represented in the lungs, while points falling below the lower bound were deemed under-represented in the lungs. All described analyses were conducted in R (v 3.5.3).

Results:

Metadata Description

Forty-eight calves were included in this study. Among these animals, nine stayed healthy throughout the whole study period (referred to as A9). Nasal swabs (NS), nasopharyngeal swabs (NPS), and bronchoalveolar lavage (BAL) samples were collected from each animal upon arrival to the feedlot (Arrival). Calves were inspected daily for signs of BRD. Ultimately, 20 calves were diagnosed with BRD (BRD) on different days, and nasal swabs, nasopharyngeal swabs and BAL samples were collected from both these calves and paired healthy controls (Control) from the same pen on the day of diagnosis. In total, 198 samples were sequenced, generating 4,477,093 sequence reads with an average of 22,727 OTUs per sample. The relative abundance of common pathogens that were detected by each sampling method (NS, NPS, BAL) in animals of each health status group (Arrival, BRD, Control) are listed below in Table 1.

TABLE 1
Relative abundance of common pathogens in healthy and BRD animals

| Common | NS | | | NPS | | | BAL | | |
|---|---|---|---|---|---|---|---|---|---|
| pathogens | Arrival | BRD | Control | Arrival | BRD | Control | Arrival | BRD | Control |
| Otu1_g_Mycoplasma | 0.392% | 2.657% | 2.437% | 27.775% | 23.924% | 25.489% | 0.652% | 5.690% | 2.349% |
| Otu3_g_Mycoplasma | 4.509% | 10.412% | 4.377% | 9.552% | 9.963% | 8.195% | 5.938% | 1.474% | 4.317% |
| Otu9_g_Mycoplasma | 0.000% | 0.913% | 3.744% | 0.182% | 3.084% | 16.979% | 0.119% | 10.592% | 9.689% |
| Otu27_g_Mycoplasma | 0.000% | 0.041% | 0.000% | 0.000% | 0.126% | 0.276% | 0.001% | 0.958% | 0.113% |
| Otu36_g_Mycoplasma | 0.042% | 1.152% | 0.628% | 0.001% | 0.007% | 0.059% | 0.000% | 0.029% | 0.016% |
| Otu367_g_Mycoplasma | 0.002% | 0.003% | 0.011% | 0.016% | 0.015% | 0.015% | 0.000% | 0.001% | 0.000% |
| Otu411_g_Mycoplasma | 0.003% | 0.030% | 0.006% | 0.000% | 0.016% | 0.009% | 0.002% | 0.000% | 0.002% |
| Otu474_g_Mycoplasma | 0.001% | 0.000% | 0.002% | 0.067% | 0.000% | 0.000% | 0.000% | 0.000% | 0.000% |
| Otu845_g_Mycoplasma | 0.000% | 0.000% | 0.002% | 0.000% | 0.003% | 0.014% | 0.000% | 0.000% | 0.000% |
| Otu2011_g_Mycoplasma | 0.000% | 0.000% | 0.000% | 0.000% | 0.002% | 0.000% | 0.000% | 0.000% | 0.000% |
| Otu2044_g_Mycoplasma | 0.000% | 0.002% | 0.000% | 0.000% | 0.000% | 0.000% | 0.002% | 0.000% | 0.000% |
| Otu2057_g_Mycoplasma | 0.000% | 0.000% | 0.000% | 0.001% | 0.000% | 0.001% | 0.000% | 0.001% | 0.000% |
| Otu2221_g_Mycoplasma | 0.000% | 0.001% | 0.004% | 0.000% | 0.000% | 0.000% | 0.000% | 0.000% | 0.000% |
| Otu2694_g_Mycoplasma | 0.000% | 0.000% | 0.000% | 0.000% | 0.000% | 0.003% | 0.000% | 0.000% | 0.000% |
| Otu2910_g_Mycoplasma | 0.001% | 0.001% | 0.000% | 0.000% | 0.000% | 0.000% | 0.002% | 0.000% | 0.000% |
| Otu3060_g_Mycoplasma | 0.000% | 0.000% | 0.000% | 0.000% | 0.000% | 0.000% | 0.000% | 0.000% | 0.000% |
| Otu3904_g_Mycoplasma | 0.000% | 0.000% | 0.000% | 0.000% | 0.000% | 0.002% | 0.000% | 0.000% | 0.000% |
| Otu4814_g_Mycoplasma | 0.000% | 0.000% | 0.000% | 0.000% | 0.000% | 0.000% | 0.000% | 0.000% | 0.000% |
| Otu2_g_Mannheimia | 1.045% | 9.514% | 7.563% | 7.924% | 20.234% | 11.819% | 1.017% | 6.178% | 1.149% |
| Otu434_g_Mannheimia | 0.009% | 0.003% | 0.018% | 0.011% | 0.005% | 0.001% | 0.000% | 0.002% | 0.000% |
| Otu815_g_Mannheimia | 0.007% | 0.011% | 0.003% | 0.000% | 0.000% | 0.000% | 0.000% | 0.001% | 0.000% |
| Otu1549_g_Mannheimia | 0.001% | 0.000% | 0.000% | 0.001% | 0.000% | 0.001% | 0.000% | 0.000% | 0.000% |
| Otu1573_g_Mannheimia | 0.003% | 0.000% | 0.000% | 0.002% | 0.001% | 0.004% | 0.000% | 0.000% | 0.000% |
| Otu4235_g_Mannheimia | 0.000% | 0.000% | 0.000% | 0.000% | 0.000% | 0.000% | 0.000% | 0.000% | 0.001% |
| Otu5_g_Moraxella | 5.677% | 5.046% | 7.131% | 16.567% | 7.251% | 1.875% | 2.324% | 0.333% | 0.725% |
| Otu8_g_Moraxella | 8.680% | 5.087% | 9.638% | 0.125% | 0.214% | 0.882% | 1.390% | 0.100% | 0.161% |
| Otu18_g_Moraxella | 0.226% | 4.676% | 1.660% | 0.085% | 0.390% | 0.026% | 0.000% | 0.010% | 0.010% |
| Otu21_f_Moraxellaceae | 0.004% | 1.304% | 1.389% | 0.000% | 0.001% | 0.003% | 0.000% | 0.016% | 0.003% |
| Otu22_f_Moraxellaceae | 1.872% | 0.337% | 2.346% | 0.002% | 0.010% | 0.206% | 0.026% | 0.056% | 0.000% |
| Otu115_g_Moraxella | 0.232% | 0.023% | 0.202% | 0.000% | 0.000% | 0.005% | 0.000% | 0.000% | 0.000% |
| Otu149_g_Moraxella | 0.252% | 0.006% | 0.002% | 0.005% | 0.000% | 0.000% | 0.027% | 0.000% | 0.000% |
| Otu509_f_Moraxellaceae | 0.043% | 0.004% | 0.006% | 0.000% | 0.000% | 0.000% | 0.000% | 0.000% | 0.000% |
| Otu646_g_Moraxella | 0.030% | 0.003% | 0.005% | 0.000% | 0.000% | 0.001% | 0.002% | 0.006% | 0.000% |
| Otu664_g_Moraxella | 0.013% | 0.035% | 0.010% | 0.000% | 0.000% | 0.001% | 0.000% | 0.000% | 0.002% |
| Otu707_f_Moraxellaceae | 0.012% | 0.003% | 0.001% | 0.001% | 0.000% | 0.005% | 0.034% | 0.000% | 0.000% |
| Otu871_f_Moraxellaceae | 0.006% | 0.003% | 0.007% | 0.000% | 0.000% | 0.000% | 0.000% | 0.000% | 0.000% |
| Otu981_g_Moraxella | 0.007% | 0.001% | 0.000% | 0.000% | 0.000% | 0.000% | 0.025% | 0.000% | 0.011% |
| Otu1403_g_Moraxella | 0.003% | 0.000% | 0.000% | 0.000% | 0.000% | 0.000% | 0.000% | 0.000% | 0.000% |
| Otu1491_g_Moraxella | 0.004% | 0.000% | 0.000% | 0.008% | 0.003% | 0.002% | 0.000% | 0.000% | 0.000% |
| Otu1567_f_Moraxellaceae | 0.005% | 0.009% | 0.003% | 0.000% | 0.000% | 0.000% | 0.000% | 0.000% | 0.000% |
| Otu1704_f_Moraxellaceae | 0.009% | 0.003% | 0.001% | 0.000% | 0.000% | 0.000% | 0.000% | 0.007% | 0.000% |
| Otu1758_g_Moraxella | 0.001% | 0.000% | 0.000% | 0.001% | 0.000% | 0.000% | 0.005% | 0.000% | 0.000% |
| Otu2026_f_Moraxellaceae | 0.001% | 0.000% | 0.000% | 0.000% | 0.000% | 0.000% | 0.014% | 0.000% | 0.000% |
| Otu2129_g_Moraxella | 0.001% | 0.001% | 0.005% | 0.000% | 0.000% | 0.000% | 0.000% | 0.000% | 0.000% |
| Otu2174_f_Moraxellaceae | 0.000% | 0.001% | 0.001% | 0.000% | 0.000% | 0.000% | 0.000% | 0.000% | 0.000% |
| Otu2219_f_Moraxellaceae | 0.001% | 0.001% | 0.000% | 0.000% | 0.000% | 0.000% | 0.001% | 0.000% | 0.000% |
| Otu2326_g_Moraxella | 0.001% | 0.001% | 0.002% | 0.000% | 0.000% | 0.000% | 0.000% | 0.000% | 0.000% |
| Otu2495_g_Moraxella | 0.001% | 0.002% | 0.000% | 0.000% | 0.000% | 0.000% | 0.000% | 0.000% | 0.000% |
| Otu2499_g_Moraxella | 0.000% | 0.001% | 0.001% | 0.000% | 0.000% | 0.000% | 0.000% | 0.000% | 0.000% |
| Otu2501_g_Moraxella | 0.002% | 0.001% | 0.000% | 0.000% | 0.000% | 0.000% | 0.000% | 0.000% | 0.000% |
| Otu2617_f_Moraxellaceae | 0.002% | 0.000% | 0.000% | 0.000% | 0.000% | 0.000% | 0.000% | 0.000% | 0.000% |
| Otu2687_f_Moraxellaceae | 0.002% | 0.000% | 0.000% | 0.000% | 0.000% | 0.000% | 0.000% | 0.000% | 0.000% |
| Otu2724_g_Moraxella | 0.001% | 0.001% | 0.002% | 0.000% | 0.000% | 0.000% | 0.000% | 0.000% | 0.000% |
| Otu2770_g_Moraxella | 0.003% | 0.001% | 0.001% | 0.000% | 0.000% | 0.000% | 0.000% | 0.000% | 0.000% |
| Otu2787_f_Moraxellaceae | 0.002% | 0.000% | 0.000% | 0.000% | 0.000% | 0.000% | 0.000% | 0.000% | 0.000% |
| Otu2865_f_Moraxellaceae | 0.002% | 0.000% | 0.000% | 0.000% | 0.000% | 0.000% | 0.000% | 0.000% | 0.000% |
| Otu2888_g_Moraxella | 0.000% | 0.001% | 0.000% | 0.000% | 0.000% | 0.000% | 0.000% | 0.000% | 0.000% |
| Otu3162_f_Moraxellaceae | 0.002% | 0.000% | 0.001% | 0.000% | 0.000% | 0.001% | 0.000% | 0.000% | 0.000% |
| Otu3327_g_Moraxella | 0.000% | 0.001% | 0.000% | 0.000% | 0.000% | 0.000% | 0.000% | 0.000% | 0.000% |
| Otu3530_g_Moraxella | 0.000% | 0.001% | 0.001% | 0.000% | 0.000% | 0.000% | 0.000% | 0.000% | 0.000% |
| Otu3542_f_Moraxellaceae | 0.000% | 0.000% | 0.000% | 0.000% | 0.000% | 0.000% | 0.000% | 0.000% | 0.000% |
| Otu3588_g_Moraxella | 0.001% | 0.000% | 0.001% | 0.000% | 0.000% | 0.000% | 0.002% | 0.000% | 0.000% |
| Otu3638_g_Moraxella | 0.002% | 0.000% | 0.000% | 0.001% | 0.000% | 0.000% | 0.000% | 0.000% | 0.000% |
| Otu3743_f_Moraxellaceae | 0.003% | 0.000% | 0.000% | 0.000% | 0.000% | 0.000% | 0.000% | 0.000% | 0.000% |
| Otu3748_g_Moraxella | 0.000% | 0.000% | 0.000% | 0.000% | 0.000% | 0.000% | 0.000% | 0.000% | 0.000% |
| Otu3755_g_Moraxella | 0.001% | 0.000% | 0.000% | 0.000% | 0.000% | 0.000% | 0.000% | 0.000% | 0.000% |
| Otu3866_f_Moraxellaceae | 0.002% | 0.001% | 0.000% | 0.000% | 0.000% | 0.000% | 0.000% | 0.000% | 0.000% |
| Otu3870_g_Moraxella | 0.002% | 0.001% | 0.000% | 0.000% | 0.000% | 0.000% | 0.000% | 0.000% | 0.000% |
| Otu4029_f_Moraxellaceae | 0.001 | 0.001% | 0.000% | 0.000% | 0.000% | 0.001% | 0.000% | 0.000% | 0.000% |
| Otu4273_g_Moraxella | 0.001% | 0.000% | 0.000% | 0.000% | 0.000% | 0.000% | 0.000% | 0.000% | 0.000% |
| Otu4352_f_Moraxellaceae | 0.000% | 0.000% | 0.001% | 0.000% | 0.000% | 0.000% | 0.000% | 0.000% | 0.000% |
| Otu4434_f_Moraxellaceae | 0.001% | 0.000% | 0.000% | 0.000% | 0.000% | 0.000% | 0.000% | 0.000% | 0.000% |
| Otu4510_f_Moraxellaceae | 0.001% | 0.000% | 0.000% | 0.000% | 0.000% | 0.000% | 0.000% | 0.000% | 0.000% |
| Otu4517_g_Moraxella | 0.000% | 0.000% | 0.001% | 0.004% | 0.000% | 0.000% | 0.000% | 0.000% | 0.000% |
| Otu4542_g_Moraxella | 0.000% | 0.001% | 0.001% | 0.000% | 0.000% | 0.000% | 0.000% | 0.000% | 0.000% |
| Otu4543_g_Moraxella | 0.000% | 0.000% | 0.001% | 0.000% | 0.000% | 0.000% | 0.000% | 0.000% | 0.000% |
| Otu4556_g_Moraxella | 0.000% | 0.001% | 0.001% | 0.000% | 0.000% | 0.000% | 0.000% | 0.000% | 0.000% |
| Otu4598_g_Moraxella | 0.000% | 0.000% | 0.000% | 0.000% | 0.000% | 0.000% | 0.000% | 0.000% | 0.000% |
| Otu4603_f_Moraxellaceae | 0.000% | 0.000% | 0.000% | 0.000% | 0.000% | 0.000% | 0.000% | 0.000% | 0.000% |
| Otu4607_f_Moraxellaceae | 0.000% | 0.000% | 0.000% | 0.000% | 0.000% | 0.000% | 0.000% | 0.000% | 0.000% |
| Otu4764_f_Moraxellaceae | 0.001% | 0.000% | 0.000% | 0.000% | 0.000% | 0.000% | 0.001% | 0.000% | 0.000% |
| Otu6_f_Pasteurellaceae | 4.395% | 15.014% | 12.126% | 0.879% | 4.264% | 1.306% | 4.414% | 0.362% | 0.464% |
| Otu7_g_Pasteurella | 1.350% | 3.260% | 2.264% | 5.390% | 5.156% | 0.871% | 0.265% | 1.269% | 0.568% |
| Otu50_f_Pasteurellaceae | 0.025% | 0.022% | 0.014% | 0.029% | 0.001% | 0.005% | 0.597% | 0.402% | 1.845% |
| Otu91_f_Pasteurellaceae | 0.046% | 0.143% | 0.230% | 0.002% | 0.000% | 0.000% | 0.000% | 0.046% | 0.013% |
| Otu424_f_Pasteurellaceae | 0.000% | 0.003% | 0.001% | 0.002% | 0.000% | 0.064% | 0.018% | 0.001% | 0.016% |
| Otu762_f_Pasteurellaceae | 0.000% | 0.009% | 0.003% | 0.000% | 0.006% | 0.000% | 0.000% | 0.000% | 0.000% |
| Otu865_f_Pasteurellaceae | 0.009% | 0.001% | 0.001% | 0.002% | 0.000% | 0.002% | 0.008% | 0.000% | 0.002% |
| Otu1049_f_Pasteurellaceae | 0.000% | 0.002% | 0.000% | 0.000% | 0.000% | 0.000% | 0.002% | 0.001% | 0.093% |
| Otu1382_f_Pasteurellaceae | 0.001% | 0.002% | 0.002% | 0.005% | 0.000% | 0.000% | 0.003% | 0.000% | 0.000% |
| Otu1591_f_Pasteurellaceae | 0.000% | 0.006% | 0.000% | 0.000% | 0.001% | 0.000% | 0.000% | 0.000% | 0.000% |
| Otu1752_g_Pasteurella | 0.001% | 0.002% | 0.004% | 0.000% | 0.001% | 0.000% | 0.000% | 0.000% | 0.000% |
| Otu1811_f_Pasteurellaceae | 0.000% | 0.000% | 0.000% | 0.001% | 0.000% | 0.000% | 0.003% | 0.000% | 0.000% |
| Otu1902_f_Pasteurellaceae | 0.000% | 0.000% | 0.000% | 0.001% | 0.000% | 0.010% | 0.007% | 0.001% | 0.000% |
| Otu1954_f_Pasteurellaceae | 0.004% | 0.002% | 0.000% | 0.000% | 0.000% | 0.000% | 0.003% | 0.006% | 0.000% |
| Otu1988_f_Pasteurellaceae | 0.000% | 0.000% | 0.000% | 0.001% | 0.000% | 0.004% | 0.002% | 0.000% | 0.000% |
| Otu2028_f_Pasteurellaceae | 0.002% | 0.003% | 0.002% | 0.000% | 0.000% | 0.000% | 0.000% | 0.001% | 0.000% |
| Otu2037_f_Pasteurellaceae | 0.000% | 0.002% | 0.002% | 0.000% | 0.001% | 0.000% | 0.000% | 0.000% | 0.000% |
| Otu2086_f_Pasteurellaceae | 0.000% | 0.006% | 0.000% | 0.000% | 0.000% | 0.000% | 0.000% | 0.000% | 0.000% |
| Otu2194_f_Pasteurellaceae | 0.000% | 0.002% | 0.003% | 0.001% | 0.000% | 0.001% | 0.001% | 0.000% | 0.000% |
| Otu2538_f_Pasteurellaceae | 0.000% | 0.001% | 0.001% | 0.000% | 0.000% | 0.000% | 0.000% | 0.000% | 0.000% |
| Otu2699_f_Pasteurellaceae | 0.000% | 0.002% | 0.001% | 0.000% | 0.000% | 0.000% | 0.001% | 0.000% | 0.000% |
| Otu2701_f_Pasteurellaceae | 0.000% | 0.000% | 0.001% | 0.000% | 0.000% | 0.000% | 0.000% | 0.000% | 0.000% |
| Otu2732_g_Pasteurella | 0.000% | 0.001% | 0.000% | 0.000% | 0.000% | 0.000% | 0.000% | 0.000% | 0.000% |
| Otu2780_f_Pasteurellaceae | 0.000% | 0.000% | 0.000% | 0.000% | 0.000% | 0.000% | 0.004% | 0.000% | 0.000% |
| Otu3406_g_Pasteurella | 0.000% | 0.001% | 0.001% | 0.000% | 0.000% | 0.000% | 0.000% | 0.000% | 0.000% |
| Otu3573_g_Pasteurella | 0.000% | 0.000% | 0.000% | 0.000% | 0.000% | 0.000% | 0.000% | 0.009% | 0.000% |
| Otu3597_f_Pasteurellaceae | 0.000% | 0.000% | 0.000% | 0.000% | 0.000% | 0.000% | 0.000% | 0.000% | 0.000% |
| Otu4520_f_Pasteurellaceae | 0.000% | 0.000% | 0.000% | 0.000% | 0.000% | 0.000% | 0.001% | 0.000% | 0.000% |
| Otu4524_g_Pasteurella | 0.000% | 0.000% | 0.000% | 0.000% | 0.000% | 0.000% | 0.000% | 0.000% | 0.000% |
| Otu4960_f_Pasteurellaceae | 0.000% | 0.001% | 0.000% | 0.000% | 0.000% | 0.000% | 0.000% | 0.000% | 0.000% |
| Otu12_g_Histophilus | 0.006% | 2.538% | 1.118% | 0.019% | 3.830% | 11.840% | 0.214% | 0.345% | 0.329% |
| Otu2145_g_Histophilus | 0.000% | 0.000% | 0.000% | 0.000% | 0.000% | 0.007% | 0.000% | 0.000% | 0.000% |
| Otu3025_g_Histophilus | 0.000% | 0.001% | 0.001% | 0.000% | 0.000% | 0.001% | 0.000% | 0.000% | 0.005% |
| Otu4958_g_Histophilus | 0.000% | 0.001% | 0.000% | 0.000% | 0.000% | 0.000% | 0.000% | 0.000% | 0.000% |

The Biogeography of the Bovine Respiratory Microbiome

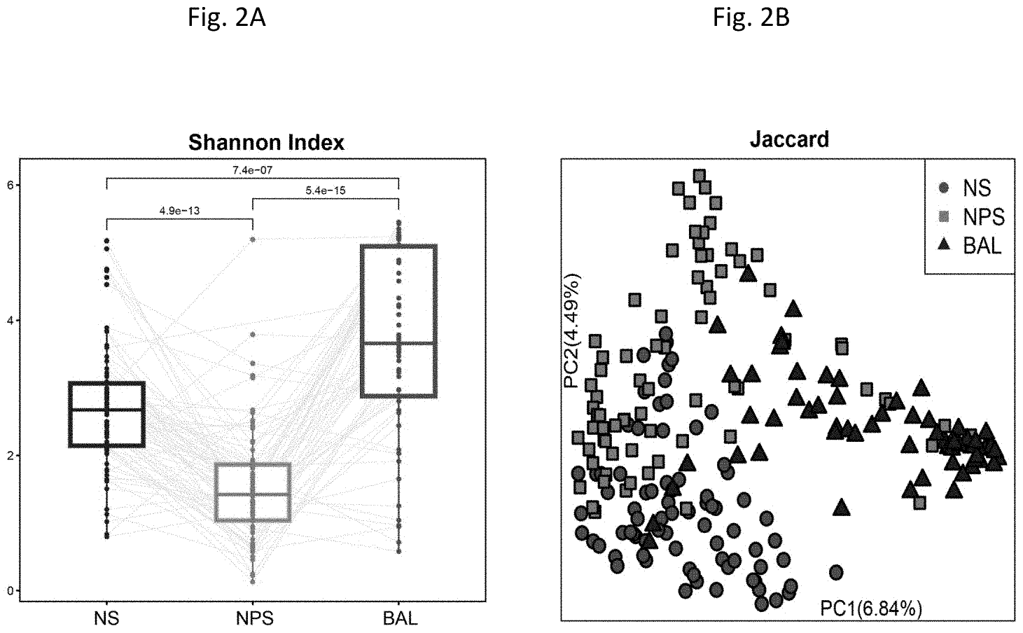

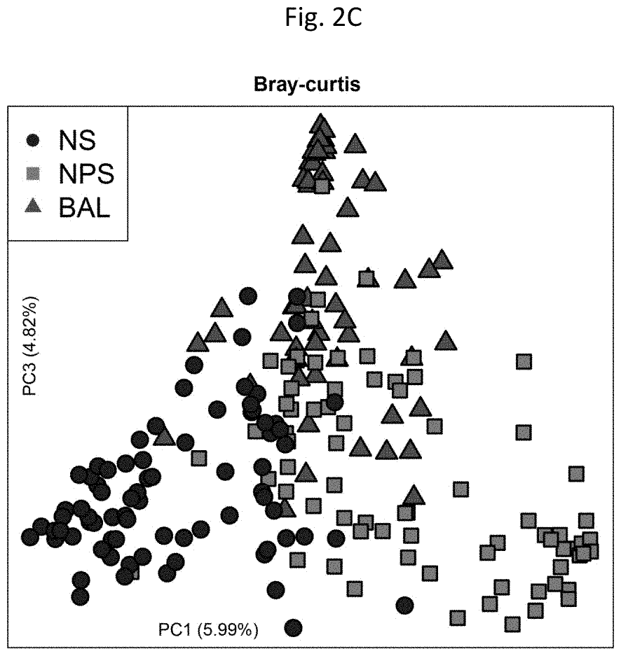

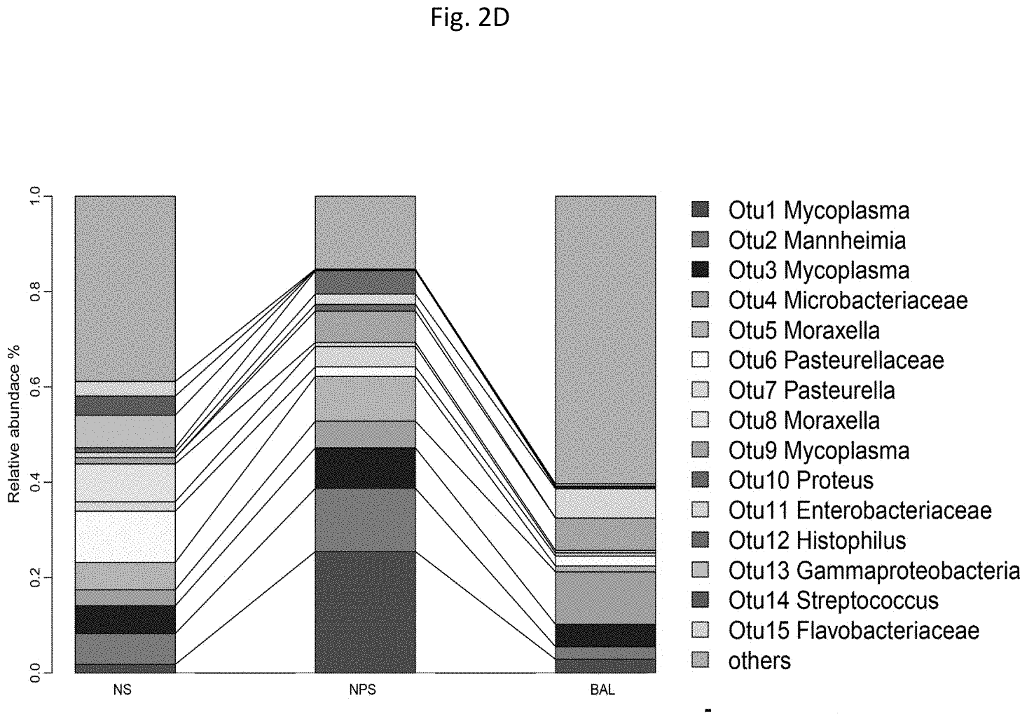

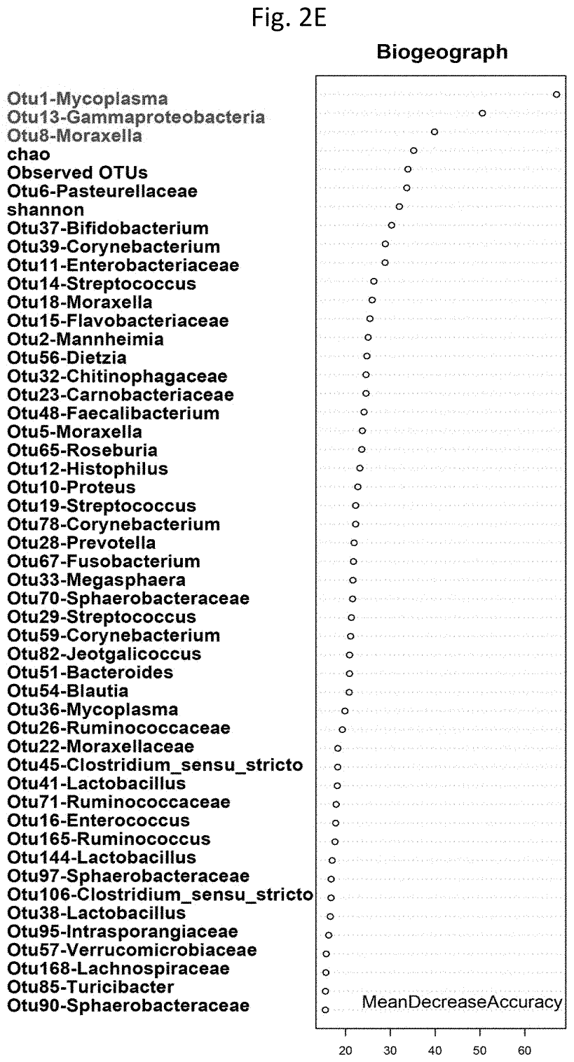

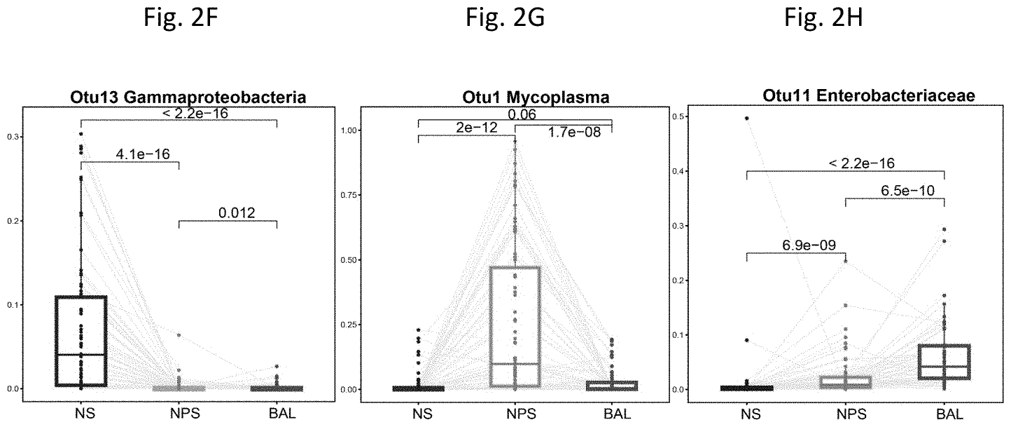

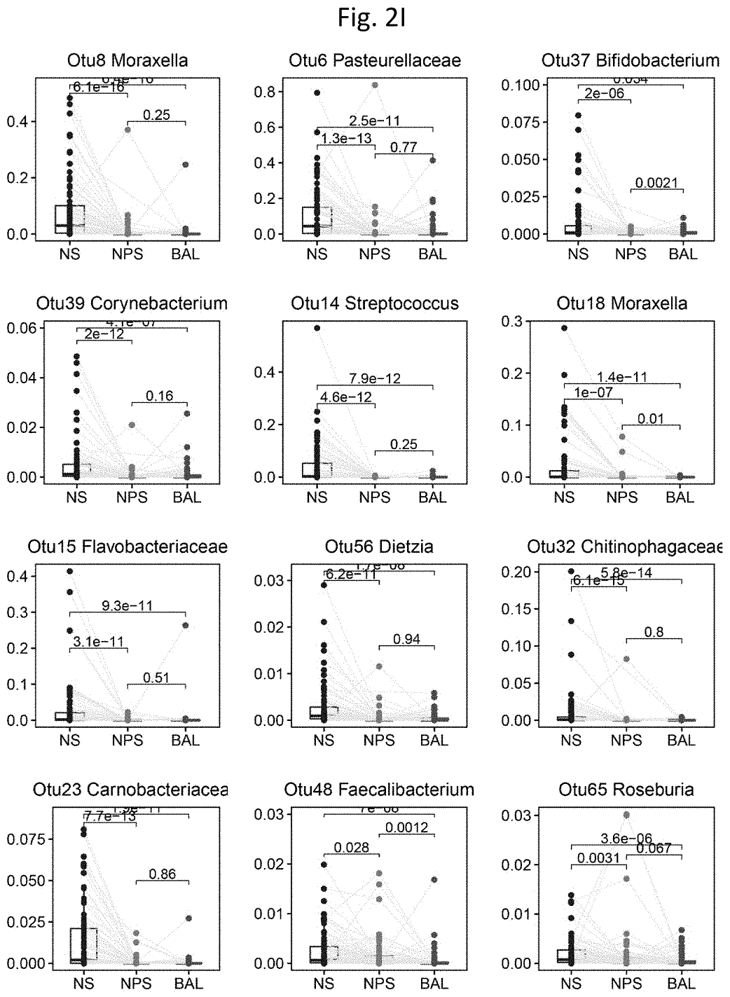

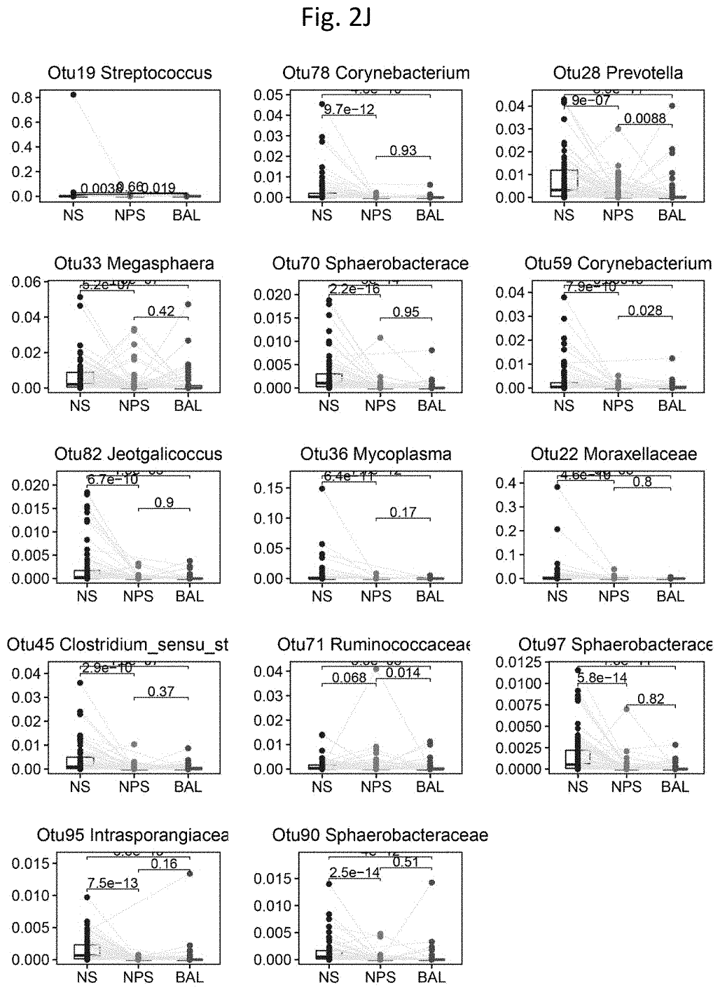

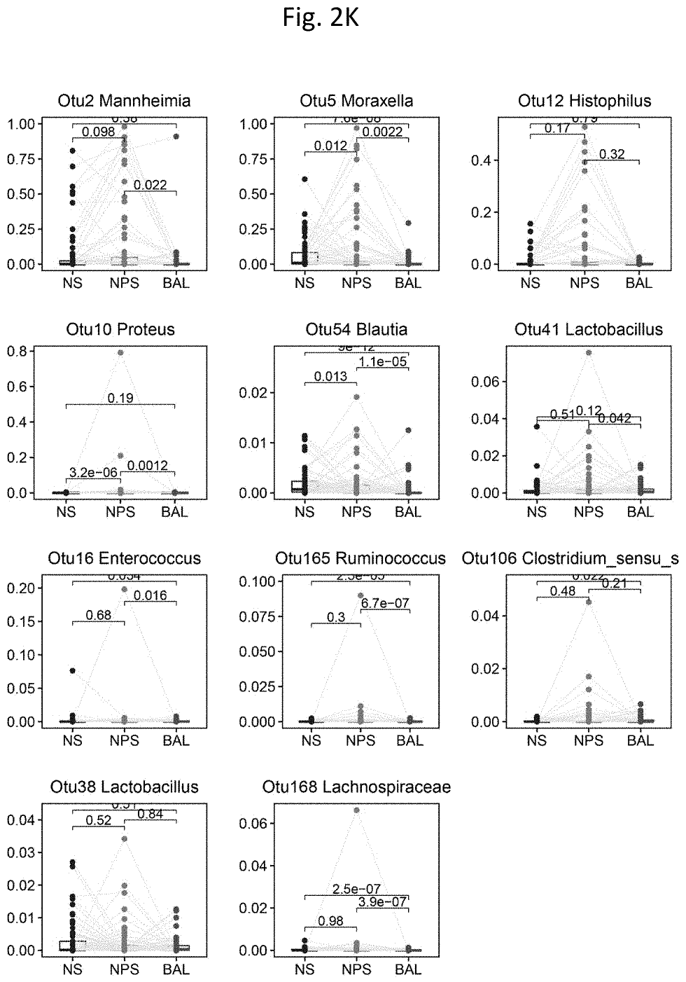

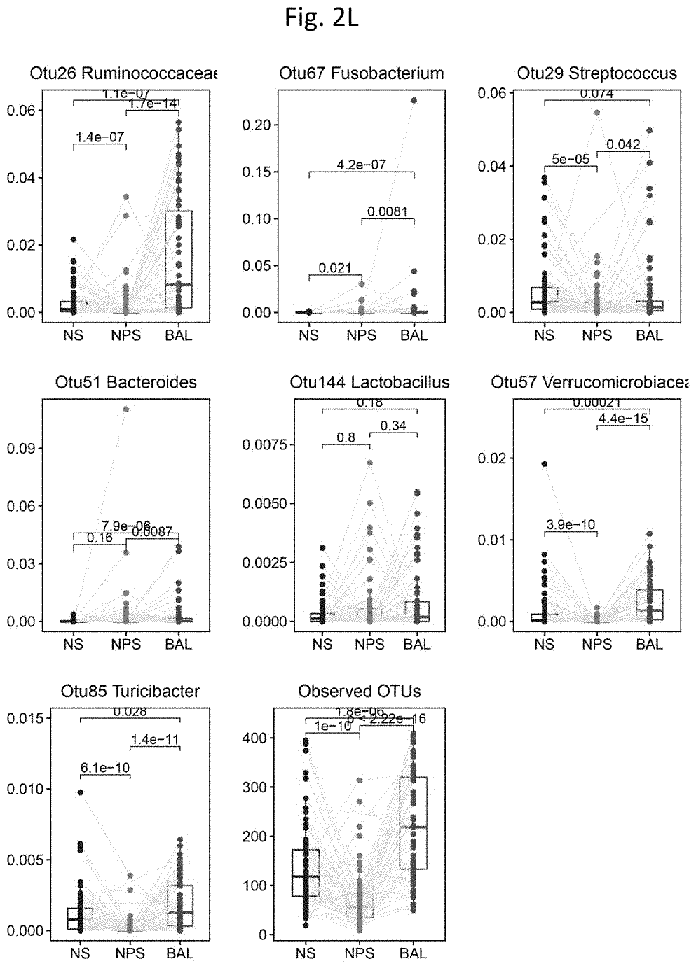

Examination of the biogeography of the bovine respiratory microbiome at the community level revealed significant differences in alpha diversity measures between the three niches. BAL samples had the greatest microbiome diversity followed by NS samples, while NPS samples possessed the least microbial diversity (FIG. 2A). Principal coordinate analysis (PCoA) based on Jaccard distance revealed distinct clusters, indicating that the different niches of the bovine respiratory system harbor different microbiomes (FIG. 2B, ANalysis Of SIMilarity, ANOSIM, Jaccard R-value: NS-NPS=0.35, NS-BAL=0.66 and NPS-BAL=0.51). The PCoA plot based on Bray-Curtis distance supported the conclusion that there is a significant difference in microbiome structure between the three respiratory niches (FIG. 2C, ANOSIM, R-value: NS-NPS=0.37, NS-BAL=0.53 and NPS-BAL=0.33). Moreover, the top bacterial OTUs were niche specific. While these top OTUs were detected at some level in all three niches, they were differentially distributed between the niches (FIG. 2D). In general, NS samples had a higher abundance of OTU6-Pasteurellaceae, OTU8-Moraxella, OTU13-Gammaproteobacteria, OTU14-Streptococcus, and OTU15-Flavobacteriaceae. NPS samples were enriched for OTU1-Mycoplasma, OTU5-Moraxella and OTU12-Histophilus. BAL samples were enriched for OTU11-Enterobacteriaceae. However, a similar abundance of OTU3-Mycoplasma was detected in all three niches, and OTU9-Mycoplasma was detected at similar levels in NPS and BAL.

To identify microbial signatures that distinguish the three niches, random forest modeling was performed using alpha diversity and the top 500 OTUs as input, as described previously (Kong et al. 2016). The top 50 most predictive features were identified and ranked based on mean decrease accuracy (FIG. 2E). OTUs associated with common BRD pathogens (e.g., Mannheimia, Pasteurella, Mycoplasma, and Histophilus) were among the top features that differentiate the three niches. The relative abundance of signature OTUs of the NS microbiome are shown in FIG. 2F-2I. An unclassified OTU (OTU13) belonging to Gammaproteobacteria was found to be enriched in NS samples and was ranked second by the random forest model (FIG. 2F). Moraxella appeared to be specifically enriched in NS samples, as several OTUs including OTU8 (FIG. 2I), OTU18 (FIG. 2I) and OTU22 (FIG. 2J) belonging to this genus were detected and over-represented in NS samples. Corynebacterium is another NS signature bacterium, and several OTUs (OTU39, OTU59 and OTU78, FIG. 2I-2J) of this genus were significantly more abundant in NS samples. Interestingly, some OTUs associated with the gastrointestinal tract were also enriched in the NS microbiome. For example, OTU37 (Bifidobacterium) and OTU48 (Faecahbacterium) were observed in 83.1% (64/77) and 80.5% (62/77) of the NS samples with an average abundance of 0.85% and 0.31%, respectively (FIG. 2J). OTUs associated with common BRD pathogens were also observed in the NS microbiome, including OTU1 (Myoplasma), OTU2 (Mannheimia), OTU6 (Pasteurellaceae), OTU12 (Histophilus) and OTU36 (Mycoplasma). However, only OTU6 and OTU36 appeared to be signatures of the NS microbiome, with higher abundance in the NS than in other niches. Other OTUs associated with BRD pathogens (e.g., OTU1 (Myoplasma), OTU2 (Mannheimia), OTU12 (Histophilus)) were signatures of the NPS microbiome (FIG. 2G, FIG. 2K). The BAL microbiome was enriched for OTUs such as Otu11 (Enterobacteriaceae), Otu26 (Ruminococcaceae) and Otu29 (Streptococcus). The distributions of additional signature OTUs among the three niches are shown in FIG. 2I-2L.

Bovine Respiratory Microbiome Signatures Predicting the Onset of BRD

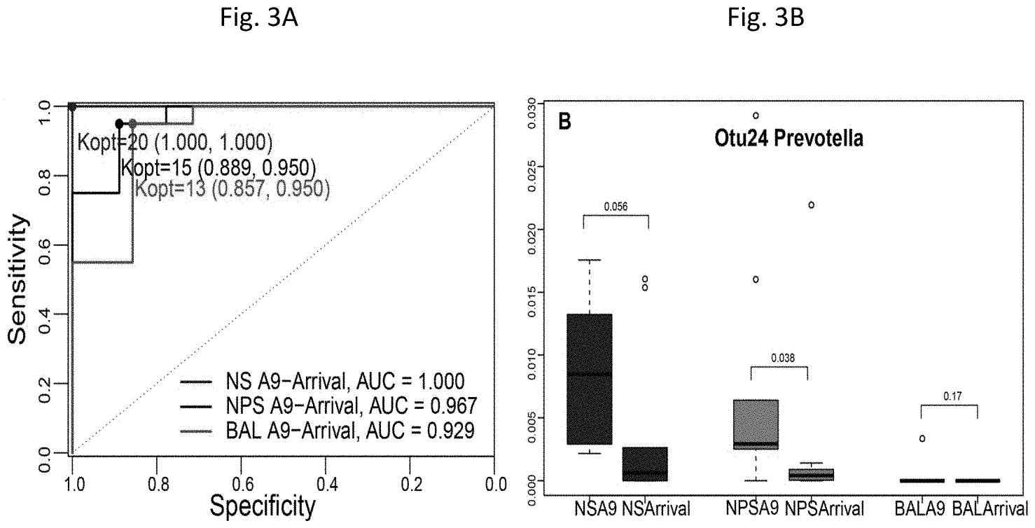

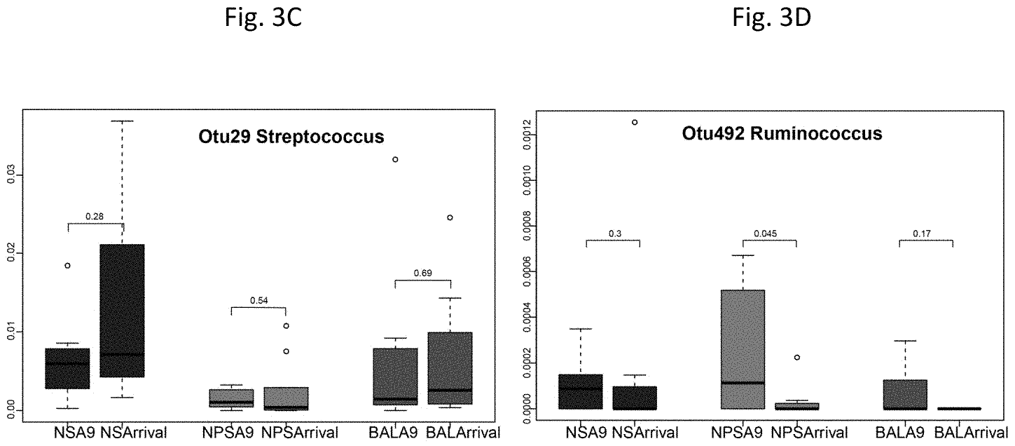

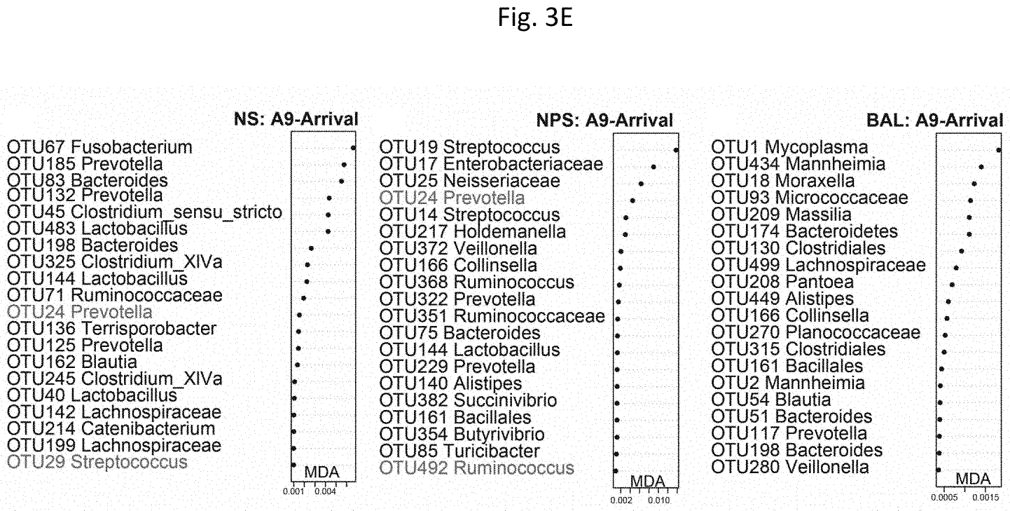

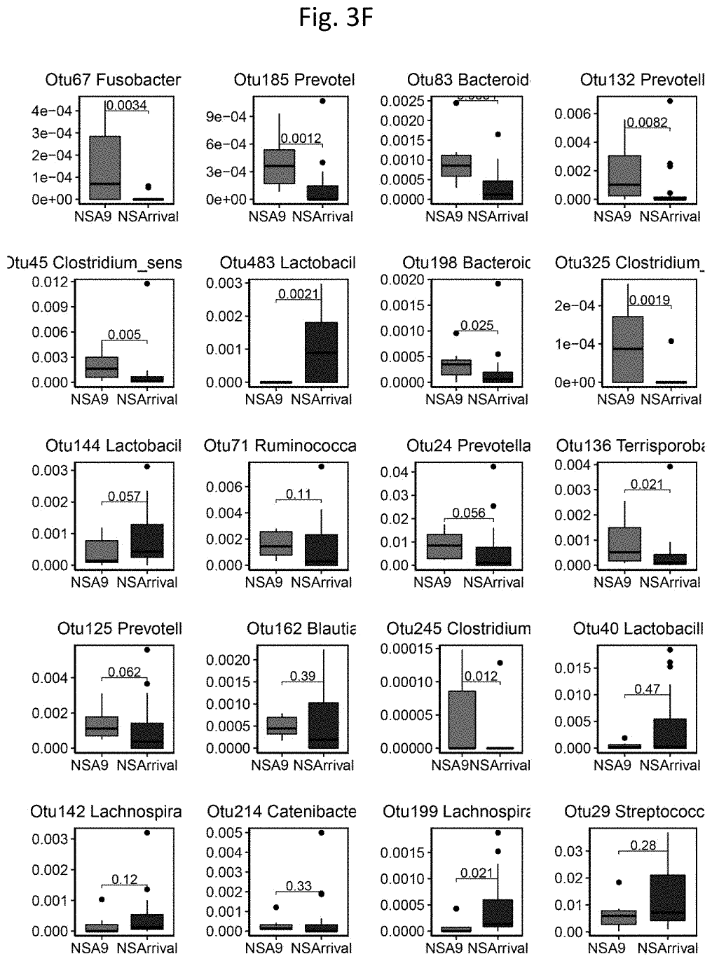

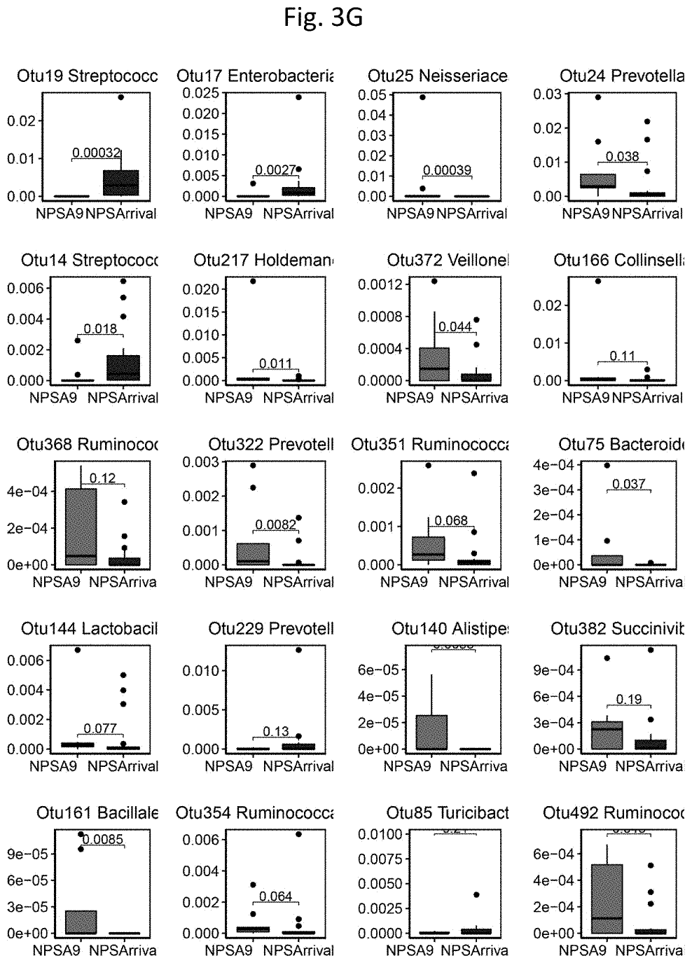

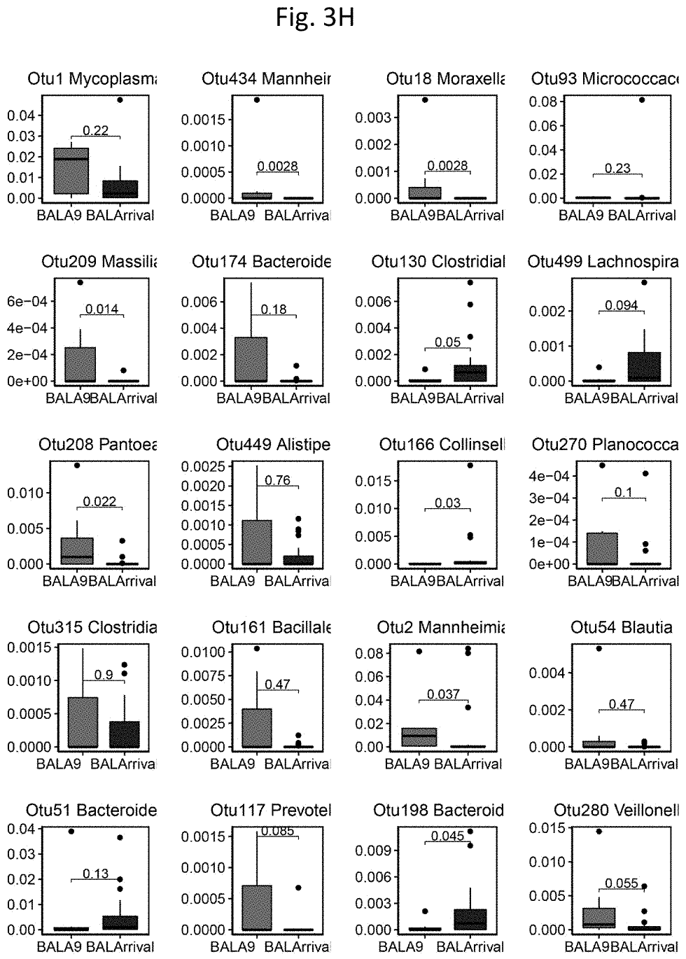

To determine if the bovine respiratory microbiome can be used to predict the onset of BRD, we analyzed the three niches within the bovine respiratory microbiome upon arrival to the feedlot (d0). We compared the microbes present in 9 calves that showed no signs of BRD throughout the study period (A9) and 20 calves that subsequently developed BRD after arrival (Arrival). Specifically, we employed a random forest machine learning model to identify OTUs present at arrival that differentiate the animals that remain healthy from those that ultimately develop BRD. The optimal model was developed based on the maximum area under the curve (AUC) using the AUC-RF algorithm. We found that data collected from all three niches could be used to accurately predict whether a given calf will develop BRD. A model based on 20 OTUs from NS sample data (FIG. 3A, Table 2) yielded an AUC of 1.0 (sensitivity=1.00, specificity=1.00), while a model based on 15 OTUs from NPS sample data (FIG. 3A, Table 3) yielded an AUC of 0.97 (sensitivity=0.95, specificity=0.90), and a model based on 13 OTUs from BAL sample data (FIG. 3A, Table 4) yielded an AUC of 0.93 (sensitivity=0.95, specificity=0.86). The top 20 OTUs from each of the three niches that distinguish healthy calves from those that developed BRD are listed in FIG. 3E. The OTUs include Fusobacterium (OTU67), Turicibacter (OTU85), and several GI-tract OTUs such as Bacteroides (OTU83 and OTU198), Prevotella (OTU24, OTU132), and Clostridium XIVa (OTU245, OTU325), which were significantly more abundant in the healthy calves (FIG. 3A-3E). Several Lactobacillus OTUs (OTU483, OTU144, and OTU40) were overrepresented in the NS samples collected from calves that developed BRD (FIG. 3F). Among the 15 predictive OTUs identified in the NPS microbiome, two OTUs from Prevotella (OTU24 and OTU322) were more abundant in healthy calves, whereas two OTUs from Streptococcus (OTU19 and OTU14) were more abundant in sick animals (FIG. 3E, 3G). Surprisingly, OTUs associated with common BRD pathogens (OTU1-Mycoplasma, OTU434-Mannheimia, and OTU18-Moraxella) were more abundant in the BAL microbiome of healthy calves (FIG. 3E, 3H).

TABLE 2
OTUs for prediction of BRD from NS samples

| Name | Source strain | Partial 16S rRNA sequence |
|---|---|---|
| SEQ ID | Fusobacterium | TACGTATGTCGCAAGCGTTATCCGGATTTATTGGGCGTA |
| NO: 1 | mortiferum | AAGCGCGTCTAGGCGGTTTGGTAAGTCTGATGTGAAAA |
| (OTU67) | strain DSM | TGCGGGGCTCAACTCCGTATTGCGTTGGAAACTGCCAA |
| | 19809 | ACTAGAGTACTGGAGAGGTGGGCGGAACTACAAGTGTA |
| | | GAGGTGAAATTCGTAGATATTTGTAGGAATGCCGATGG |
| | | GGAAGCCAGCCCACTGGACAGATACTGACGCTAAAGCG |
| | | CGAAAGCGTGGGTAGCAAACAGG |
| SEQ ID | Prevotella | TACGGAAGGTCCGGGCGTTATCCGGATTTATTGGGTTTA |
| NO: 2 | stercorea | AAGGGAGCGTAGGCCGTCTGTTAAGCGTGTTGTGAAAT |
| (OTU185) | DSM 18206 | GTCGGGGCTCAACCTGGGCATTGCAGCGCGAACTGGCA |
| | strain CB35 | GACTTGAGTGCGCGGGAAGTAGGCGGAATTCGTCGTGT |
| | | AGCGGTGAAATGCTTAGATATGACGAAGAACTCCGATT |
| | | GCGAAGGCAGCCTGCTGTAGTGCAACTGACGCTGAAGC |
| | | TCGAAAGCGTGGGTATCGAACAGG |
| SEQ ID | Bacteroides | TACGGAGGATCCGAGCGTTATCCGGATTTATTGGGTTTA |
| NO: 3 | vulgatus | AAGGGAGCGTAGATGGATGTTTAAGTCAGTTGTGAAAG |
| (OTU83) | ATCC 8482 | TTTGCGGCTCAACCGTAAAATTGCAGTTGATACTGGATA |
| | | TCTTGAGTGCAGTTGAGGCAGGCGGAATTCGTGGTGTA |
| | | GCGGTGAAATGCTTAGATATCACGAAGAACTCCGATTG |
| | | CGAAGGCAGCCTGCTAAGCTGCAACTGACATTGAGGCT |
| | | CGAAAGTGTGGGTATCAAACAGG |
| SEQ ID | Prevotella | TACGGAAGGTCCGGGCGTTATCCGGATTCATTGGGTTTA |
| NO: 4 | oris strain | AAGGGAGCGCAGGCCGCCCCTTAAGCGTGTTGTGAAAT |
| (OTU132) | JCM 12252 | GCCGCGGCTCAACCGTGGCACTGCAGCGCGAACTGGGG |
| | | GGCTTGAGTGCGCGCAGCGCAGGCGGAACTCGTGGTGT |
| | | AGCGGTGAAATGCTTAGATATCACGAAGAACTCCGATC |
| | | GCGAAGGCAGCCTGCGGGAGCGCAACTGACGCTGAGG |
| | | CTCGAAAGTGCGGGTATCGAACAGG |
| SEQ ID | Clostridium | TACGTAGGTGGCGAGCGTTGTCCGGATTTACTGGGCGT |
| NO: 5 | saudiense | AAAGGGAGCGTAGGCGGATTTTTAAGTGAGATGTGAAA |
| (OTU45) | strain JCC | TACTCGGGCTTAACCTGAGTGCTGCATTTCAAACTGGAA |
| | | GTCTAGAGTGCAGGAGAGGAGAAGGGAATTCCTAGTGT |
| | | AGCGGTGAAATGCGTAGAGATTAGGAAGAACACCAGT |
| | | GGCGAAGGCGCTTCTCTGGACTGTAACTGACGCTGAGG |
| | | CTCGAAAGCGTGGGGAGCAAACAGG |
| SEQ ID | Lactobacillus | TACGTAGGTGGCAAGCGTTGTCCGGATTTATTGGGCGT |
| NO: 6 | plantarum | AAAGCGAGCGCAGGCGGTTTTTTAAGTCTGATGTGAAA |
| (OTU483) | strain CIP | GCCTTCGGCTCAACCGAAGAAGTGCATCGGAAACTGGG |
| | 103151 | AAACTTGAGTGCAGAAGAGGACAGTGGAACTCCATGTG |
| | | TAGCGGTGAAATGCGTAGATATATGGAAGAACACCAGT |
| | | GGCGAAGGCGGCTGTCTGGTCTGTAACTGACGCTGAGG |
| | | CTCGAAAGTATGGGTAGCAAACAGG |
| SEQ ID | Bacteroides | TACGGAGGATCCGAGCGTTATCCGGATTTATTGGGTTTA |
| NO: 7 | uniformis | AAGGGAGCGTAGGCGGACGCTTAAGTCAGTTGTGAAAG |
| (OTU198) | strain JCM | TTTGCGGCTCAACCGTAAAATTGCAGTTGATACTGGGTG |
| | 5828 | TCTTGAGTACAGTAGAGGCAGGCGGAATTCGTGGTGTA |
| | | GCGGTGAAATGCTTAGATATCACGAAGAACTCCGATTG |
| | | CGAAGGCAGCTTGCTGGACTGTAACTGACGCTGATGCT |
| | | CGAAAGTGTGGGTATCAAACAGG |
| SEQ ID | [Clostridium] | TACGTAGGGGGCAAGCGTTATCCGGATTTACTGGGTGT |
| NO: 8 | clostridioforme | AAAGGGAGCGTAGACGGCGAAGCAAGTCTGAAGTGAA |
| (OTU325) | strain | AACCCAGGGCTCAACCCTGGGACTGCTTTGGAAACTGT |
| | ATCC | TTTGCTAGAGTGTCGGAGAGGTAAGTGGAATTCCTAGT |
| | 25537 | GTAGCGGTGAAATGCGTAGATATTAGGAGGAACACCAG |
| | | TGGCGAAGGCGGCTTACTGGACGATAACTGACGTTGAG |
| | | GCTCGAAAGCGTGGGGAGCAAACAGG |
| SEQ ID | Lactobacillus | TACGTAGGTGGCAAGCGTTATCCGGATTTATTGGGCGT |
| NO: 9 | mucosae | AAAGCGAGCGCAGGCGGTTTGATAAGTCTGATGTGAAA |
| (OTU144) | strain S32 | GCCTTTGGCTTAACCAAAGAAGTGCATCGGAAACTGTC |
| | | AGACTTGAGTGCAGAAGAGGACAGTGGAACTCCATGTG |
| | | TAGCGGTGGAATGCGTAGATATATGGAAGAACACCAGT |
| | | GGCGAAGGCGGCTGTCTGGTCTGCAACTGACGCTGAGG |
| | | CTCGAAAGCATGGGTAGCGAACAGG |
| SEQ ID | Gemmiger | AACGTAGGGTGCAAGCGTTGTCCGGAATTACTGGGTGT |
| NO: 10 | formicilis | AAAGGGAGCGCAGGCGGACCGGCAAGTTGGAAGTGAA |
| (OTU71) | strain X2-56 | AACCATGGGCTCAACCCATAAATTGCTTTCAAAACTGCT |
| | | GGCCTTGAGTAGTGCAGAGGTAGGTGGAATTCCCGGTG |
| | | TAGCGGTGGAATGCGTAGATATCGGGAGGAACACCAGT |
| | | GGCGAAGGCGACCTACTGGGCACCAACTGACGCTGAGG |
| | | CTCGAAAGCATGGGTAGCAAACAGG |
| SEQ ID | Prevotella | TACGGAAGGTCCGGGCGTTATCCGGATTTATTGGGTTTA |
| NO: 11 | copri DSM | AAGGGAGCGTAGGCCGGAGATTAAGCGTGTTGTGAAAT |
| (OTU24) | 18205 strain | GTAGTGGCTCAACCTCTGCACTGCAGCGCGAACTGGTC |
| | JCM 13464 | TTCTTGAGTACGCACAACGTGGGCGGAATTCGTGGTGT |
| | | AGCGGTGAAATGCTTAGATATCACGAAGAACTCCGATT |
| | | GCGAAGGCAGCTCACGGGAGCGCAACTGACGCTGAAG |
| | | CTCGAAAGTGCGGGTATCGAACAGG |
| SEQ ID | Terrisporo- | TACGTAGGGGGCTAGCGTTATCCGGATTTACTGGGCGT |
| NO: 12 | bacter | AAAGGGTGCGTAGGTGGTTTCTTAAGTCAGGAGTGAAA |
| (OTU136) | petrolearius | GGCTACGGCTCAACCGTAGTAAGCTCTTGAAACTGGGA |
| | strain | AACTTGAGTGCAGGAGAGGAAAGTGGAATTCCTAGTGT |
| | LAM0A37 | AGCGGTGAAATGCGTAGATATTAGGAGGAACACCAGTA |
| | | GCGAAGGCGGCTTTCTGGACTGTAACTGACACTGAGGC |
| | | ACGAAAGCGTGGGGAGCAAACAGG |
| SEQ ID | Prevotella | TACGGAAGGTCCGGGCGTTATCCGGATTTATTGGGTTTA |
| NO: 13 | oris strain | AAGGGAGCGTAGGCCGTCTTTTAAGCGTGTTGTGAAAT |
| (OTU125) | JCM 12252 | ACTGTCGCTCAACGACAGAGGTGCAGCGCGAACTGGGA |
| | | GACTTGAGTGCGCGGAATGCAGGCGGAATTCGTCGTGT |
| | | AGCGGTGAAATGCTTAGATATGACGAAGAACTCCGATT |
| | | GCGAAGGCAGCTTGCAGTAGCGTAACTGACGCTGAAGC |
| | | TCGAAAGTGCGGGTATCGAACAGG |
| SEQ ID | Blautia | TACGTAGGGGGCAAGCGTTATCCGGATTTACTGGGTGT |
| NO: 14 | obeum | AAAGGGAGCGTAGACGGACTGGCAAGTCTGATGTGAA |
| (OTU162) | ATCC 29174 | AGGCGGGGGCTCAACCCCTGGACTGCATTGGAAACTGT |
| | | TAGTCTTGAGTGCCGGAGAGGTAAGCGGAATTCCTAGT |
| | | GTAGCGGTGAAATGCGTAGATATTAGGAGGAACACCAG |
| | | TGGCGAAGGCGGCTTACTGGACGGTAACTGACGTTGAG |
| | | GCTCGAAAGCGTGGGGAGCAAACAGG |
| SEQ ID | [Clostridium] | TACGTAGGGGGCAAGCGTTATCCGGATTTACTGGGTGT |
| NO: 15 | scindens | AAAGGGAGCGTAGACGGCGATGCAAGCCAGATGTGAA |
| (OTU245) | strain | AGCCCGGGGCTCAACCCCGGGACTGCATTTGGAACTGC |
| | ATCC 35704 | GTGGCTGGAGTGTCGGAGAGGCAGGCGGAATTCCTAGT |
| | | GTAGCGGTGAAATGCGTAGATATTAGGAGGAACACCAG |
| | | TGGCGAAGGCGGCCTGCTGGACGATGACTGACGTTGAG |
| | | GCTCGAAAGCGTGGGGAGCAAACAGG |
| SEQ ID | Lactobacillus | TACGTAGGTGGCAAGCGTTATCCGGATTTATTGGGCGT |
| NO: 16 | caviae | AAAGCGAGCGCAGGCGGTTGCTTAGGTCTGATGTGAAA |
| (OTU40) | strain MOZM2 | GCCTTCGGCTTAACCGAAGAAGTGCATCGGAAACCGGG |
| | | CGACTTGAGTGCAGAAGAGGACAGTGGAACTCCATGTG |
| | | TAGCGGTGGAATGCGTAGATATATGGAAGAACACCAGT |
| | | GGCGAAGGCGGCTGTCTGGTCTGCAACTGACGCTGAGG |
| | | CTCGAAAGCATGGGTAGCGAACAGG |
| SEQ ID | Ruminococcus | TACGTAGGGGGCAAGCGTTATCCGGATTTACTGGGTGT |
| NO: 17 | lactaris | AAAGGGAGCGCAGACGGAACTGCAAGTCTGATGTGAA |
| (OTU142) | ATCC 29176 | AGCCCGGGGCTCAACCGCGGGACTGCATCGGATACTGT |
| | | AGATCTGGAGTGCCGGAGGGGCAAGCGGAATTCCTGGT |
| | | GTAGCGGTGAAATGCGTAGATATCAGGAGGAACACCGG |
| | | CGGCGAAGGCGGCTTGCTGGACGGCAACTGACGTTGAG |
| | | GCTCGAAAGCGTGGGGAGCAAACAGG |
| SEQ ID | Catenibacterium | TACGTAGGTGGCGAGCGTTATCCGGAATCATTGGGCGT |
| NO: 18 | mitsuokai | AAAGAGGGAGCAGGCGGCCGCAAGGGTCTGTGGTGAA |
| (OTU214) | strain DSM | AGACCGAAGCTAAACTTCGGTGAGCCATGGAAACCGGG |
| | 15897 | CGGCTAGAGTGCGGAAGAGGATCGTGGAATTCCATGTG |
| | | TAGCGGTGAAATGCGTAGATATATGGAGGAACACCAGT |
| | | GGCGAAGGCGACGGTCTGGGCCGCAACTGACGCTCATT |
| | | CCCGAAAGCGTGGGGAGCAAATAGG |
| SEQ ID | Kineothrix | TACGTAGGGGGCAAGCGTTATCCGGATTTACTGGGTGT |
| NO: 19 | alysoides | AAAGGGAGCGTAGACGGCAAGGCAAGTCTGATGTGAA |
| (OTU199) | strain | ATACCGGGGCTCAACCCCGGGACTGCATTGGAAACTGT |
| | KNHS209 | TTAGCTAGAGTGCAGGAGAGGTAAGTGGAATTCCTAGT |
| | | GTAGCGGTGAAATGCGTAGATATTAGGAGGAACACCAG |
| | | TGGCGAAGGCGGCTTACTGGACTGTAACTGACGTTGAG |
| | | GCTCGAAAGCGTGGGGAGCAAACAGG |
| SEQ ID | Streptococcus | TACGTAGGTCCCGAGCGTTGTCCGGATTTATTGGGCGTA |
| NO: 20 | pasteurianus | AAGCGAGCGCAGGCGGTTTAATAAGTCTGAAGTTAAAG |
| (OTU29) | strain CIP | GCAGTGGCTTAACCATTGTTCGCTTTGGAAACTGTTAAA |
| | 107122 | CTTGAGTGCAGAAGGGGAGAGTGGAATTCCATGTGTAG |
| | | CGGTGAAATGCGTAGATATATGGAGGAACACCGGTGGC |
| | | GAAAGCGGCTCTCTGGTCTGTAACTGACGCTGAGGCTC |
| | | GAAAGCGTGGGGAGCAAACAGG |

TABLE 3
OTUs for prediction of BRD from NPS samples

| | Source | |
|---|---|---|
| Name | strain | Partial 16S rRNA sequence |
| SEQ ID | Streptococcus | TACGTAGGTCCCGAGCGTTGTCCGGATTTATTGGGCGTA |
| NO: 21 | uberis | AAGCGAGCGCAGGCGGTTTGATAAGTCTGAAGTTAAAG |
| (OTU19) | strain JCM | GCTGTGGCTTAACCATAGTTCGCTTTGGAAACTGTCAAA |
| | 5709 | CTTGAGTGCAGAAGGGGAGAGTGGAATTCCATGTGTAG |
| | | CGGTGAAATGCGTAGATATATGGAGGAACACCGGTGGC |
| | | GAAAGCGGCTCTCTGGTCTGTAACTGACGCTGAGGCTC |
| | | GAAAGCGTGGGGAGCAAACAGG |
| SEQ ID | Salmonella | TACGGAGGGTGCAAGCGTTAATCGGAATTACTGGGCGT |
| NO: 22 | enterica | AAAGCGCACGCAGGCGGTCTGTCAAGTCGGATGTGAAA |
| (OTU17) | subsp. | TCCCCGGGCTCAACCTGGGAACTGCATTCGAAACTGGC |
| | enterica | AGGCTTGAGTCTTGTAGAGGGGGGTAGAATTCCAGGTG |
| | serovar | TAGCGGTGAAATGCGTAGAGATCTGGAGGAATACCGGT |
| | Typhimurium | GGCGAAGGCGGCCCCCTGGACAAAGACTGACGCTCAGG |
| | strain | TGCGAAAGCGTGGGGAGCAAACAGG |
| | ATCC 13311 | |
| SEQ ID | Kingella | TACGTAGGGTGCAAGCGTTAATCGGAATTACTGGGCGT |
| NO: 23 | negevensis | AAAGCGAGCGCAGACGGTTATTTAAGTCAGATGTGAAA |
| (OTU25) | strain | TCCCCGAGCTCAACTTGGGAACTGCGTTTGAAACTGGA |
| | Sch538 | TAGCTAGAGTGTGTCAGAGGGGGGTAGAATTCCACGTG |
| | | TAGCAGTGAAATGCGTAGAGATGTGGAGGAATACCGAT |
| | | GGCGAAGGCAGCCCCCTGGGATAACACTGACGTTCATG |
| | | CTCGAAAGCGTGGGTAGCAAACAGG |
| SEQ ID | Prevotella | TACGGAAGGTCCGGGCGTTATCCGGATTTATTGGGTTTA |
| NO: 11 | copri DSM | AAGGGAGCGTAGGCCGGAGATTAAGCGTGTTGTGAAAT |
| (OTU24) | 18205 strain | GTAGTGGCTCAACCTCTGCACTGCAGCGCGAACTGGTC |
| | JCM 13464 | TTCTTGAGTACGCACAACGTGGGCGGAATTCGTGGTGT |
| | | AGCGGTGAAATGCTTAGATATCACGAAGAACTCCGATT |
| | | GCGAAGGCAGCTCACGGGAGCGCAACTGACGCTGAAG |
| | | CTCGAAAGTGCGGGTATCGAACAGG |
| SEQ ID | Streptococcus | TACGTAGGTCCCGAGCGTTGTCCGGATTTATTGGGCGTA |
| NO: 24 | pluranimalium | AAGCGAGCGCAGGTGGTTTAATAAGTCTGAAGTTAAAG |
| (OTU14) | strain | GCATTGGCTCAACCAATGTACGCTTTGGAAACTGTTAA |
| | T70 | ACTTGAGTGCAGAAGGGGAGAGTGGAATTCCATGTGTA |
| | | GCGGTGAAATGCGTAGATATATGGAGGAACACCGGTGG |
| | | CGAAAGCGGCTCTCTGGTCTGTAACTGACACTGAGGCT |
| | | CGAAAGCGTGGGTAGCGAACAGG |
| SEQ ID | Holdemanella | TACGTAGGTGGCGAGCGTTATCCGGAATGATTGGGCGT |
| NO: 25 | biformis | AAAGGGTGCGTAGGTGGCAGATCAAGTCTGGAGTAAAA |
| (OTU217) | strain DSM | GGTATGGGCTCAACCCGTACTGGCTCTGGAAACTGATC |
| | 3989 | AGCTAGAGAACAGAAGAGGACGGCGGAACTCCATGTG |
| | | TAGCGGTAAAATGCGTAGATATATGGAAGAACACCGGT |
| | | GGCGAAGGCGGCCGTCTGGTCTGGATTCTGACACTGAA |
| | | GCACGAAAGCGTGGGGAGCAAATAGG |
| SEQ ID | Veillonella | TACGTAGGTGGCAAGCGTTGTCCGGAATTATTGGGCGT |
| NO: 26 | dispar strain | AAAGCGCGCGCAGGCGGATTTGTCAGTCTGTCTTAAAA |
| (OTU372) | ATCC | GTTCGGGGCTTAACCCCGTGAGGGGATGGAAACTACAA |
| | 17748 | ATCTAGAGTATCGGAGAGGAAAGTGGAATTCCTAGTGT |
| | | AGCGGTGAAATGCGTAGATATTAGGAAGAACACCGGTG |
| | | GCGAAGGCGACTTTCTGGACGAAAACTGACGCTGAGGC |
| | | GCGAAAGCCAGGGGAGCGAACGGG |
| SEQ ID | Collinsella | TACGTAGGGGGCGAGCGTTATCCGGATTCATTGGGCGT |
| NO: 27 | aerofaciens | AAAGCGCGCGTAGGCGGCCCGGCAGGCCGGGGGTCGA |
| (OTU166) | strain JCM | AGCGGGGGGCTCAACCCCCCGAAGCCCCCGGAACCTCC |
| | 10188 | GCGGCTTGGGTCCGGTAGGGGAGGGTGGAACACCCGGT |
| | | GTAGCGGTGGAATGCGCAGATATCGGGTGGAACACCGG |
| | | TGGCGAAGGCGGCCCTCTGGGCCGAGACCGACGCTGAG |
| | | GCGCGAAAGCTGGGGGAGCGAACAGG |
| SEQ ID | Ruminococcus | TACGTAGGGAGCAAGCGTTGTCCGGATTTACTGGGTGT |
| NO: 28 | bromii | AAAGGGTGCGTAGGCGGCTTTGCAAGTCAGATGTGAAA |
| (OTU368) | strain | TCTATGGGCTCAACCCATAAACTGCATTTGAAACTGTAG |
| | ATCC 27255 | AGCTTGAGTGAAGTAGAGGCAGGCGGAATTCCCCGTGT |
| | | AGCGGTGAAATGCGTAGAGATGGGGAGGAACACCAGT |
| | | GGCGAAGGCGGCCTGCTGGGCTTTAACTGACGCTGAGG |
| | | CACGAAAGCGTGGGTAGCAAACAGG |
| SEQ ID | Prevotella | TACGGAAGGTCCGGGCGTTATCCGGATTTATTGGGTTTA |
| NO: 29 | oris strain | AAGGGAGCGTAGGCCGGAGATTAAGCGTGTTGTGAAAT |
| (OTU322) | JCM 12252 | GCCGCGGCTCAACCGTGGCACTGCAGCGCGAACTGGTT |
| | | TTCTTGAGTGCGCGCGACGCAGGCGGAATTCGTGGTGT |
| | | AGCGGTGAAATGCTTAGATATCACGAGGAACCCCGATT |
| | | GCGAAGGCAGCTTGCGGGAGCGCAACTGACGCTGAAGC |
| | | TCGAAGGTGCGGGTATCGAACAGG |
| SEQ ID | Fournierella | AACGTAGGTGGCAAGCGTTGTCCGGAATTACTGGGTGT |
| NO: 30 | massiliensis | AAAGGGAGCGCAGGCGGAAGGACAAGTTGGAAGTGAA |
| (OTU351) | strain AT2 | ACCCACGGGCTCAACCCGTGAACTGCTTTCAAAACTGTT |
| | | TTTCTTGAGTGGTGTAGAGGTAGGCGGAATTCCCGGTGT |
| | | AGCGGTGGAATGCGTAGATATCGGGAGGAACACCAGTG |
| | | GCGAAGGCGGCCTACTGGGCACTAACTGACGCTGAGGC |
| | | TCGAAAGCATGGGTAGCAAACAGG |
| SEQ ID | Bacteroides | TACGGAGGATGCGAGCGTTATCCGGATTTATTGGGTTTA |
| NO: 31 | plebeius | AAGGGAGCGTAGACGGGATGTTAAGTCAGTTGTGAAAG |
| (OTU75) | DSM 17135 | GCTGCGGCTCAACCGCAGCACTGCAGTTGATACTGGCG |
| | strain M12 | TCCTTGAGTGCGGTTGAGGTGTGTGGAATTCGTGGTGTA |
| | | GCGGTGAAATGCTTAGATATCACGAAGAACTCCGATTG |
| | | CGAAGGCAGCACACTAAGCCGCTACTGACGTTGAGGCT |
| | | CGAAAGTGTGGGTATCAAACAGG |
| SEQ ID | Lactobacillus | TACGTAGGTGGCAAGCGTTATCCGGATTTATTGGGCGT |
| NO: 9 | mucosae | AAAGCGAGCGCAGGCGGTTTGATAAGTCTGATGTGAAA |
| (OTU144) | strain S32 | GCCTTTGGCTTAACCAAAGAAGTGCATCGGAAACTGTC |
| | | AGACTTGAGTGCAGAAGAGGACAGTGGAACTCCATGTG |
| | | TAGCGGTGGAATGCGTAGATATATGGAAGAACACCAGT |
| | | GGCGAAGGCGGCTGTCTGGTCTGCAACTGACGCTGAGG |
| | | CTCGAAAGCATGGGTAGCGAACAGG |
| SEQ ID | Prevotella | TACGGAAGGTCCGGGCGTTATCCGGATTTATTGGGTTTA |
| NO: 32 | copri DSM | AAGGGAGCGTAGGCCGGAGGGTAAGCGTGTTGTGAAAT |
| (OTU229) | 18205 strain | GCAGTTGCTCAACATCTGCACTGCAGCGCGAACTGTTCT |
| | JCM 13464 | CCTTGAGTGCGCAGGAAGTAGGCGGAATTCGTCGTGTA |
| | | GCGGTGAAATGCTTAGATATGACGAAGAACTCCGATTG |
| | | GGAAGCCAGCTTACTGTAGCGCCACTGACGCTGATGCT |
| | | CGAAAGTGCGGGTATCGAACAGG |
| SEQ ID | Alistipes | TACGGAGGATCCAAGCGTTATCCGGATTTATTGGGTTTA |
| NO: 33 | finegoldii | AAGGGTGCGTAGGCGGATTAGTAAGTTAGAGGTGAAAG |
| (OTU140) | strain DSM | CTCGGGGCTCAACTCCGGAACTGCCTCTGATACTGCTGA |
| | 17242 | TCTAGAGAGTAGATGCGGTAGGCGGAATGTATGGTGTA |
| | | GCGGTGAAATGCTTAGAGATCATACAGAACACCGATTG |
| | | CGAAGGCAGCTTACCAAACTATATCTGACGTTGAGGCA |
| | | CGAAAGCGTGGGGAGCAAACAGG |

TABLE 4
OTUs for prediction of BRD from BAL samples

| | Source | |
|---|---|---|
| Name | strain | Partial 16S rRNA sequence |
| SEQ ID | Mycoplasma | TACATAGGTCGCAAGCGTTATCCGGAATTATTGGGCGT |
| NO: 34 | dispar | AAAGCGTCCGTAGGTTTTTTGTTAAGTTTAAGGTTAAAT |
| (OTU1) | strain 462/2 | GCTAAAGCTCAACTTTAGTCCGCTTTAGATACTGGCAAA |
| | | ATAGAATTATGAAGAGGTTAGCGGAATTCCTAGTGGAG |
| | | CGGTGGAATGCGTAGATATTAGGAAGAACACCAATAGG |
| | | CGAAGGCAGCTAACTGGTCATATATTGACACTAAGGGA |
| | | CGAAAGCGTGGGGAGCAAACAGG |
| SEQ ID | Mannheimia | TACAGAGGGTGCAAGCGTTAATCGGAATAACTGGGCGT |
| NO: 35 | haemolytica | AAAGGGCACGCAGGCGGTTGTTTAAGTGAGGTGTGAAA |
| (OTU434) | strain | GCCCCGGGCTTAACCTGGGAATTGCATTTCAGACTGAA |
| | NCTC 9380 | CAACTAGAGTACTTTAGGGAGGGGTAGAATTCCACGTG |
| | | TAGCGGTGAAATGCGTAGAGATGTGGAGGAATACCGAA |
| | | GGCGAAGGCAGCCCCTTGGAAATGTACTGACGCTCATG |
| | | TGCGAAAGCGTGGGGAGCAAACAGG |
| SEQ ID | Moraxella | TACAGAGGGTGCAAGCGTTAATCGGAATTACTGGGCGT |
| NO: 36 | caviae strain | AAAGCGAGCGTAGGTGGTTGTTTAAGTCAGATGTGAAA |
| (OTU18) | GP11 | GCCCCGGGCTTAACCTGGGAACTGCATCTGATACTGGA |
| | | CAACTAGAGTAGGTGAGAGGGAAGTAGAATTCCAGGTG |
| | | TAGCGGTGAAATGCGTAGAGATCTGGAGGAATACCGAT |
| | | GGCGAAGGCAGCTTCCTGGCATCATACTGACACTGAGG |
| | | TTCGAAAGCGTGGGTAGCAAACAGG |
| SEQ ID | Micrococcus | TACGTAGGGTGCGAGCGTTATCCGGAATTATTGGGCGT |
| NO: 37 | luteus | AAAGAGCTCGTAGGCGGTTTGTCGCGTCTGTCGTGAAA |
| (OTU93) | strain | GTCCGGGGCTTAACCCCGGATCTGCGGTGGGTACGGGC |
| | NCTC 2665 | AGACTAGAGTGCAGTAGGGGAGACTGGAATTCCTGGTG |
| | | TAGCGGTGGAATGCGCAGATATCAGGAGGAACACCGAT |
| | | GGCGAAGGCAGGTCTCTGGGCTGTAACTGACGCTGAGG |
| | | AGCGAAAGCATGGGGAGCGAACAGG |
| SEQ ID | Massilia | TACGTAGGGTGCAAGCGTTAATCGGAATTACTGGGCGT |
| NO: 38 | agri strain | AAAGCGTGCGCAGGCGGTTTTGTAAGTCTGACGTGAAA |
| (OTU209) | K-3-1 | GCCCCGGGCTCAACCTGGGAATTGCGTTGGAGACTGCA |
| | | AGGCTTGAATCTGGCAGAGGGGGGTAGAATTCCACGTG |
| | | TAGCAGTGAAATGCGTAGAGATGTGGAGGAACACCGAT |
| | | GGCGAAGGCAGCCCCCTGGGTCAAGATTGACGCTCATG |
| | | CACGAAAGCGTGGGGAGCAAACAGG |
| SEQ ID | Terrimonas | TACGGAGGGTGCAAGCATTATCCGGATTTATTGGGTTTA |
| NO: 39 | lutea strain | AAGGGTGCGTAGGTGGCTAGGTAAGTCAGTGGTGAAAT |
| (OTU174) | DY | CCCCAAGCTTAACTTGGGAACTGCCATTGATACTATCTA |
| | | GCTTGAATGTTGAGGAGGTGAGCAGAATATATCATGTA |
| | | GCGGTGAAATGCTCAGATATGATATAGAATACCGATTG |
| | | CGAAGGCCGCTCACTACACAACTATTGACACTGAGGCA |
| | | CGAAAGCGTGGGGATCAAACAGG |
| SEQ ID | Alkalibacter | TACGTAGGGGGCAAGCGTTGTCCGGAATGACTGGGCGT |
| NO: 40 | saccharofer | AAAGGGCGTGTAGGCGGCTTTTTAAGTGTGAAGTGAAA |
| (OTU130) | mentans | GTCCTGCTTTCAAGGTGGGAACTGCTTTGCAAACTGGA |
| | strain Z- | GAGCTTGAGTGCGGAAGAGGTAAGTGGAATTCCCAGTG |
| | 79820 | TAGCGGTGAAATGCGTAGAGATTGGGAGGAACACCAGT |
| | | GGCGAAGGCGACTTACTGGGCCGTAACTGACGCTGAGG |
| | | CGCGAAAGCGTGGGGAGCGAACAGG |
| SEQ ID | [Clostridium] | TACGTAGGGGGCAAGCGTTATCCGGATTTACTGGGTGT |
| NO: 41 | glycyrrhizin | AAAGGGAGCGTAGACGGAAGAGCAAGTCTGATGTGAA |
| (OTU499) | ilyticum | AACCCGGGGCTCAACCCCGGGACTGCATTGGAAACTGT |
| | strain ZM35 | TTTTCTGGAGTGTCGGAGAGGTAAGCGGAATTCCTAGT |
| | | GTAGCGGTGAAATGCGTAGATATTAGGAGGAACACCAG |
| | | TGGCGAAGGCGGCTTACTGGACGATCACTGACGTTGAG |
| | | GCTCGAAAGCGTGGGGAGCAAACAGG |
| SEQ ID | Flavobacterium | TACGGAGGGTGCAAGCGTTAATCGGAATTACTGGGCGT |
| NO: 42 | acidificum | AAAGCGCACGCAGGCGGTCTGTTAAGTCAGATGTGAAA |
| (OTU208) | strain LMG | TCCCCGGGCTTAACCTGGGAACTGCATTTGAAACTGGC |
| | 8364 | AGGCTTGAGTCTCGTAGAGGGGGGTAGAATTCCAGGTG |
| | | TAGCGGTGAAATGCGTAGAGATCTGGAGGAATACCGGT |
| | | GGCGAAGGCGGCCCCCTGGACGAAGACTGACGCTCAGG |
| | | TGCGAAAGCGTGGGGAGCAAACAGG |
| SEQ ID | Alistipes | TACGGAGGATCCAAGCGTTATCCGGATTTATTGGGTTTA |
| NO: 43 | putredinis | AAGGGTGCGTAGGCGGTTTGGTAAGTTAGAGGTGAAAT |
| (OTU449) | strain JCM | TTCAGGGCTCAACCTTGACATTGCCTCTGATACTGTCGA |
| | 16772 | ACTAGAGAGTAGTTGCTGTGGGCGGAATGTATGGTGTA |
| | | GCGGTGAAATGCTTAGAGATCATACAGAACACCGATTG |
| | | CGAAGGCAGCTCACAAAACTATATCTGACGTTGAGGCA |
| | | CGAAAGCGTGGGTAGCAAACAGG |
| SEQ ID | Collinsella | TACGTAGGGGGCGAGCGTTATCCGGATTCATTGGGCGT |
| NO: 28 | aerofaciens | AAAGCGCGCGTAGGCGGCCCGGCAGGCCGGGGGTCGA |
| (OTU166) | strain JCM | AGCGGGGGGCTCAACCCCCCGAAGCCCCCGGAACCTCC |
| | 10188 | GCGGCTTGGGTCCGGTAGGGGAGGGTGGAACACCCGGT |
| | | GTAGCGGTGGAATGCGCAGATATCGGGTGGAACACCGG |
| | | TGGCGAAGGCGGCCCTCTGGGCCGAGACCGACGCTGAG |
| | | GCGCGAAAGCTGGGGGAGCGAACAGG |
| SEQ ID | Solibacillus | TACGTAGGTGGCAAGCGTTGTCCGGAATTATTGGGCGT |
| NO: 44 | isronensis | AAAGCGCGCGCAGGTGGTTCTTTAAGTCTGATGTGAAA |
| (OTU270) | B3W22 | GCCCCCGGCTCAACCGGGGAGGGTCATTGGAAACTGGG |
| | | GAACTTGAGTGCAGAAGAGGATAGTGGAATTCCAAGTG |
| | | TAGCGGTGAAATGCGTAGAGATTTGGAGGAACACCAGT |
| | | GGCGAAGGCGACTGTCTGGTCTGTAACTGACACTGAGG |
| | | CGCGAAAGCGTGGGGAGCAAACAGG |
| SEQ ID | Monoglobus | TACGTAGGTGGCAAGCGTTGTCCGGATTTACTGGGTGT |
| NO: 45 | pectinilyticus | AAAGGGCGTGTAGGCGGGCTGATAAGTCAGATGTGAAA |
| (OTU315) | strain 14 | TACCGGGGCTTAACCCCGGGGCTGCATTTGAAACTGTC |
| | | AGTCTTGAGTGTCGGAGAGGTAAGCGGAATTCCTAGTG |
| | | TAGCGGTGAAATGCGTAGATATTAGGAGGAACACCAGT |
| | | GGCGAAGGCGGCTTACTGGACGATAACTGACGCTGAGG |
| | | CGCGAAAGCGTGGGTAGCAAACAGG |

Longitudinal Changes of Bovine Respiratory Microbiota Upon the Onset of BRD

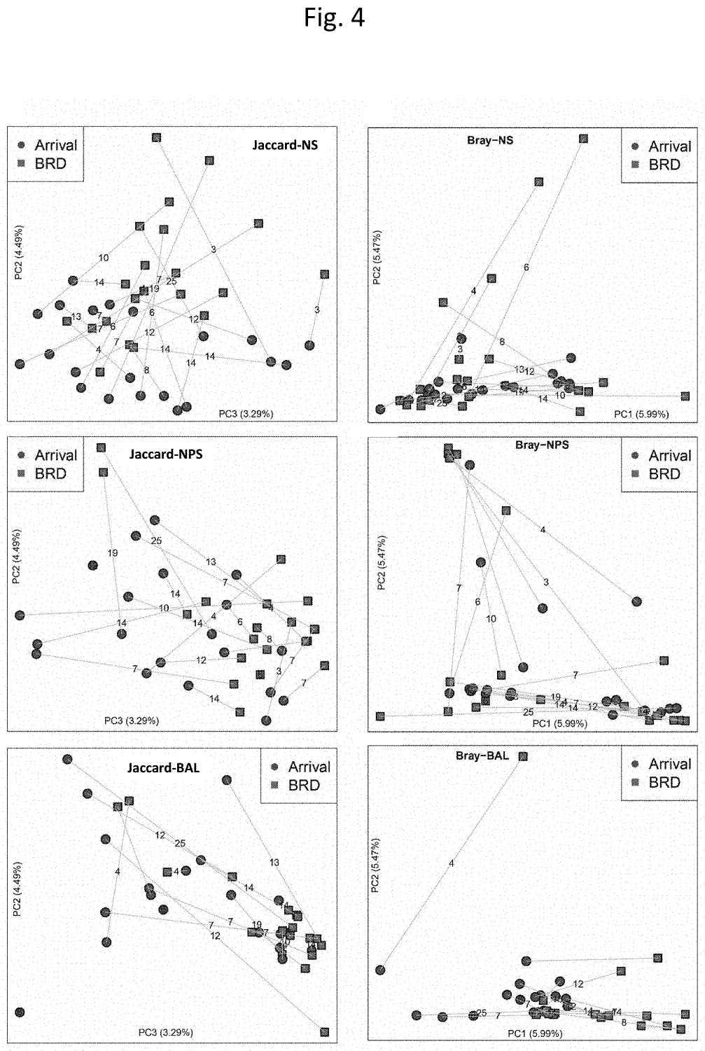

To determine how the bovine respiratory microbiome changes upon the onset of BRD, we compared the microbiota of 20 calves when they arrived the feedlot (Arrival) to when they were diagnosed with BRD (BRD). Significant changes in microbial community membership and structure were observed upon onset of BRD in all the three niches, as is demonstrated by PCoA plots based on Jaccard and Bray-Curtis distance (FIG. 4, paired t test, P<0.05, ANOSIM, NS: R=0.39, P<0.001; NPS: R value=0.12, P=0.002; BAL: R value=0.07, P=0.02).

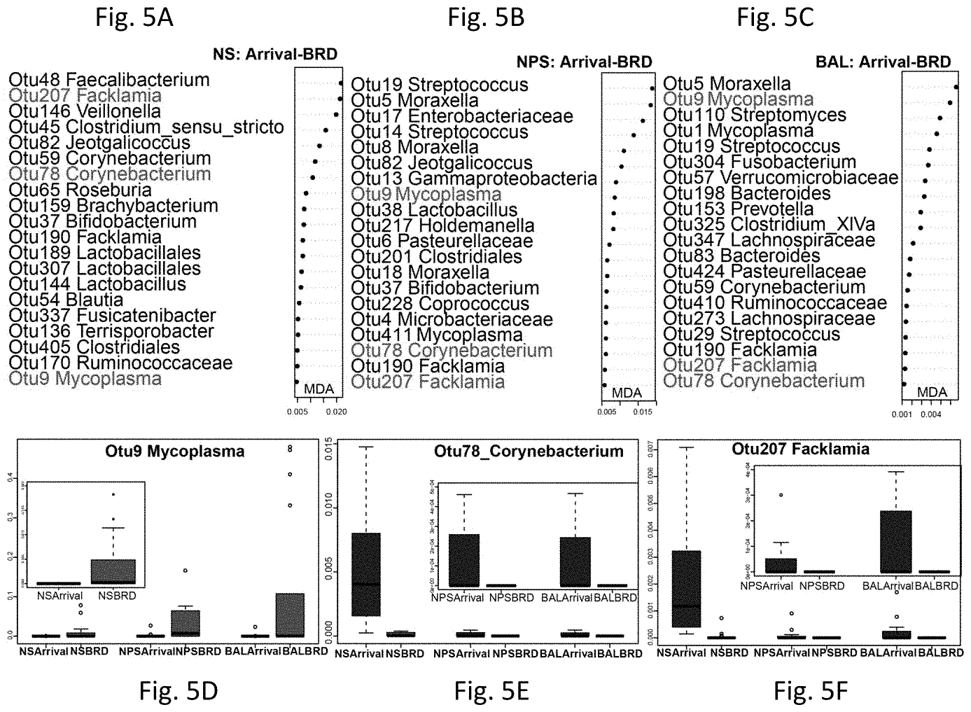

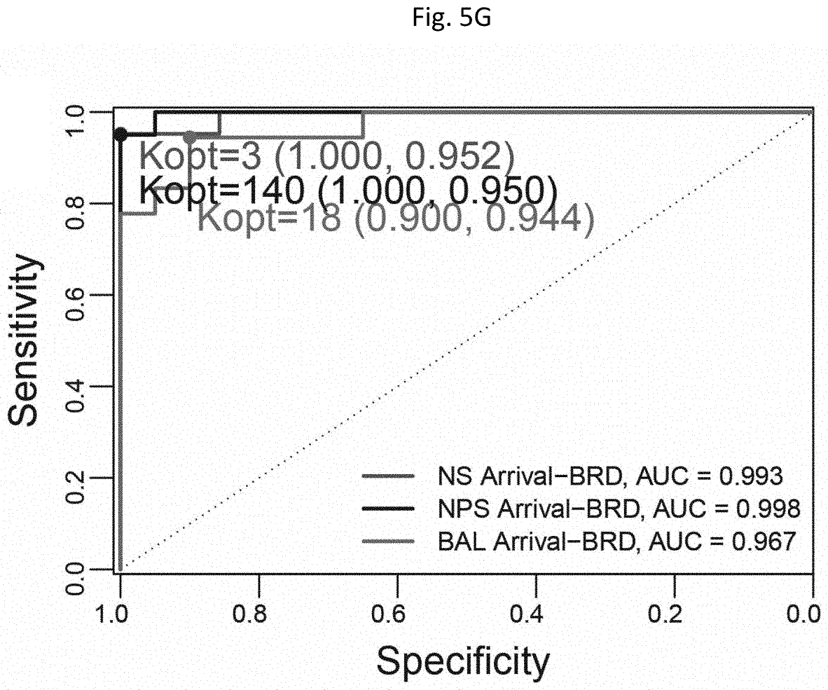

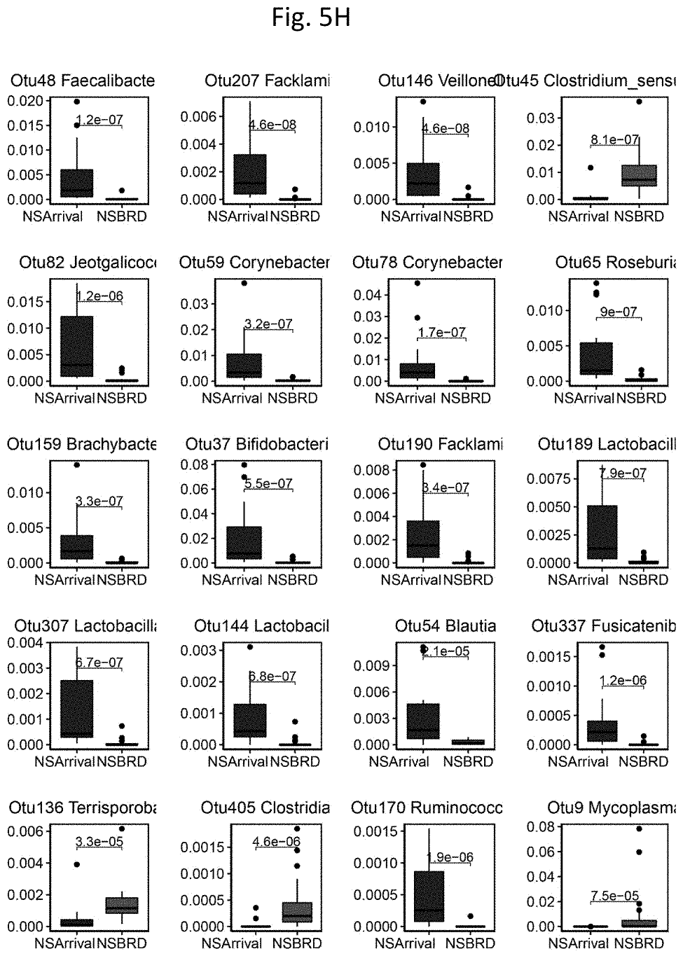

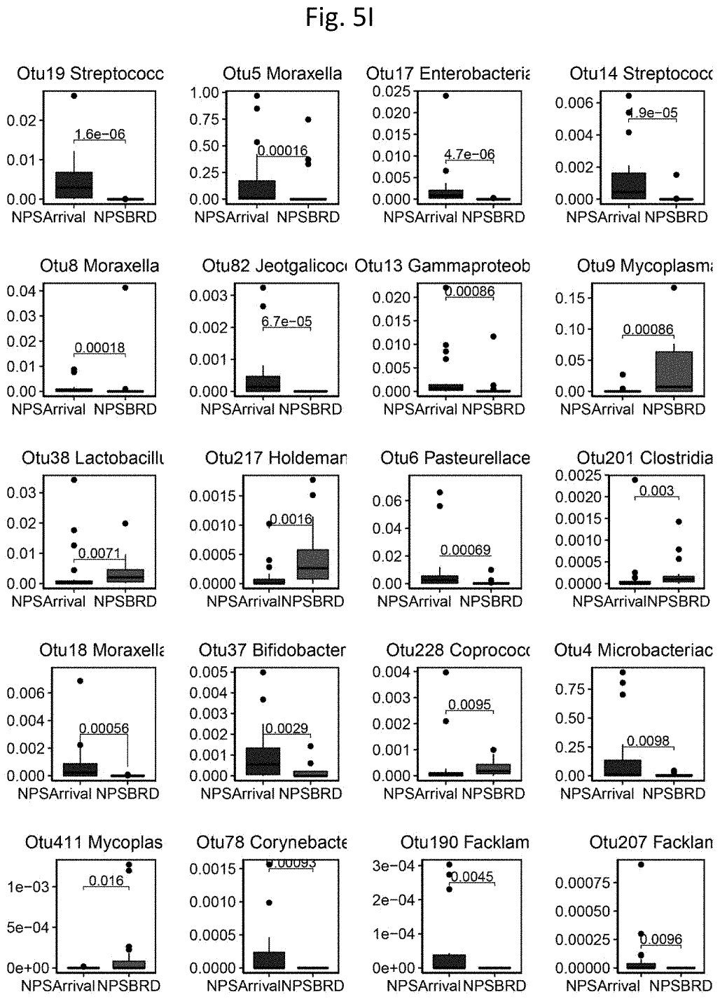

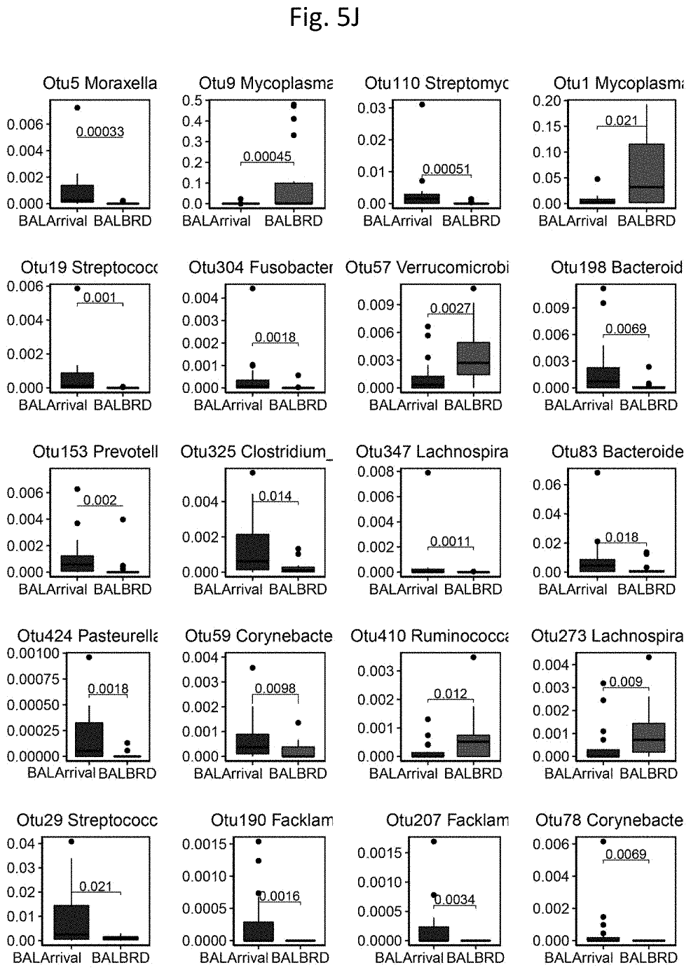

Random forest models were employed to identify bacterial features that change significantly before and during the onset of BRD in NS, NPS and BAL samples. Four features (OTU9-Mycoplasma, OTU78-Corynebacterium, OTU190-Facklamia, and OTU207-Facklamia) were shared among the three niches (FIG. 5A-5C). Though the abundance of these features was niche specific, the features showed similar dynamics upon the onset of BRD. OTU9 increased at the onset of BRD in all three niches, but especially in the lungs (FIG. 5D), while the other shared OTUs (OTU78, OTU190, and OTU207) decreased in all three niches (FIG. 5E-5I). In the nasal microbiome, three OTUs could distinguish the Arrival samples from the BRD samples with an AUC of 0.999 (OTU48-Faecalibacterium, OTU207-Facklamia, and OTU146-Veillonella). These OTUs all decreased upon the appearance of BRD symptoms (FIG. 5A, 5H). In the nasopharyngeal microbiome, 140 OTUs were needed to obtain the greatest AUC of 0.998. However, just the top eight OTUs from NPS samples could distinguish the groups with an AUC of 0.973. In these samples, OTU9, which is affiliated with Mycoplasma, increased significantly upon the onset BRD (FIG. 5B, 5I). OTU9 also increased in the lower respiratory microbiome (i.e., in BAL samples) with BRD onset, as did a second Mycoplasma-associated OTU (OTU1) (FIG. 5C, 5J). Most of the other identified features were found to decrease with the onset of BRD (FIG. 5H-5J).

Respiratory Microbiota Differentiating Healthy Calves from Those with BRD

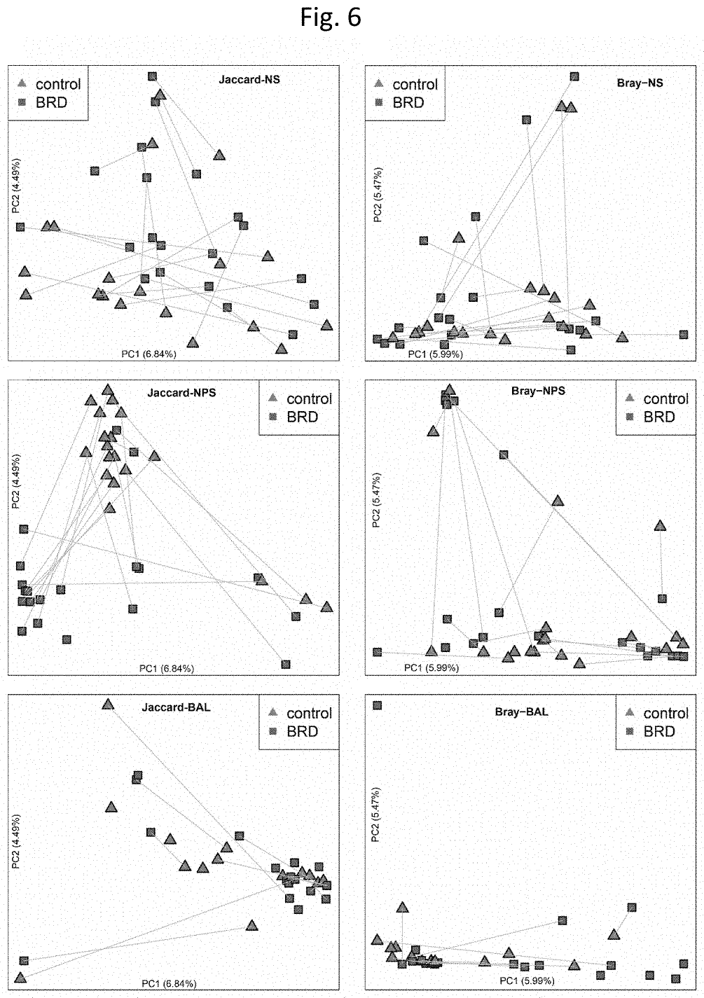

To determine if the bovine respiratory microbiome could be used to accurately diagnose BRD, we collected NS, NPS, and BAL samples from calves when they were diagnosed with BRD and compared them with samples from healthy controls without signs of BRD. Significant differences in community membership were found between the NS and NPS (but not BAL) samples collected from healthy calves and those from calves with BRD, as shown by PCoA analysis based on Jaccard distance (FIG. 6, ANOSIM, NS: R=0.23, P<0.001; NPS: R value=0.32, P<0.001; BAL: R value=0.07, P=0.116). However, we did not detect any significant difference in community structure between these groups based on Bray-Curtis distances (FIG. 6, ANOSIM, NS: R=0.11, P<0.003; NPS: R value=0.01, P<0.49; BAL: R value=0.05, P=0.14).

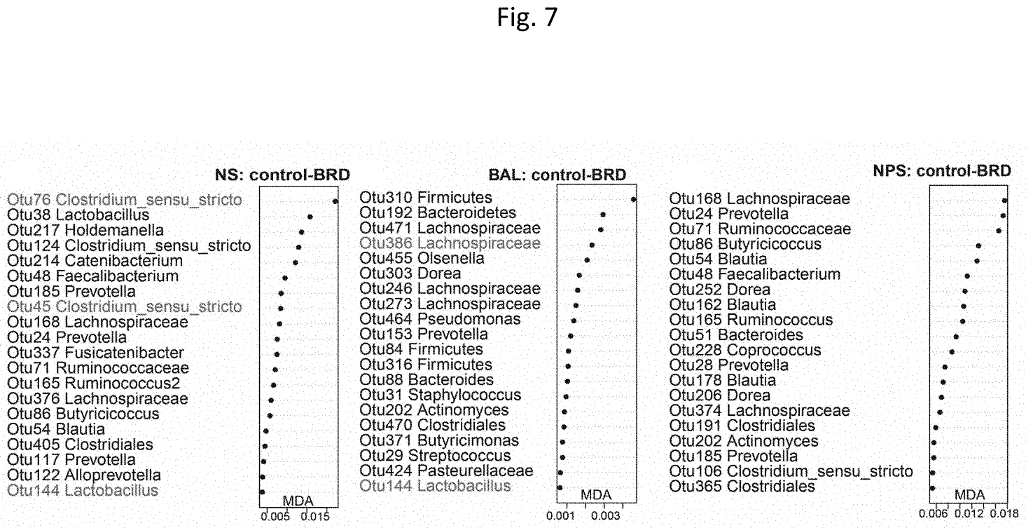

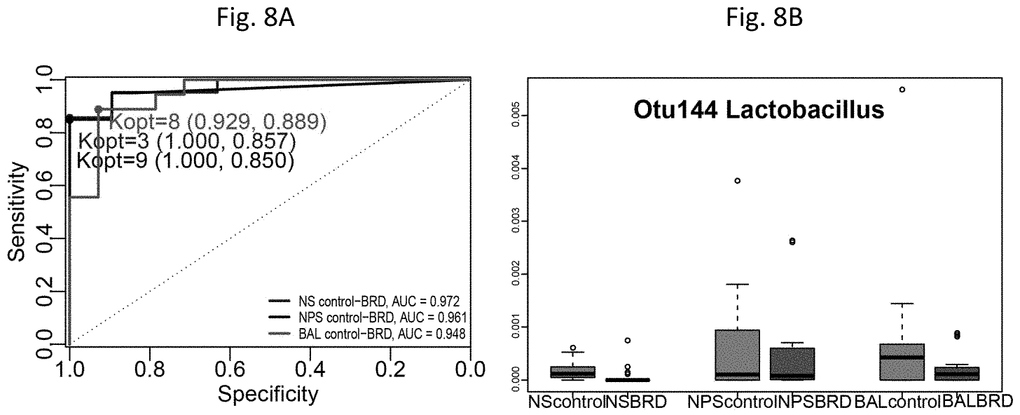

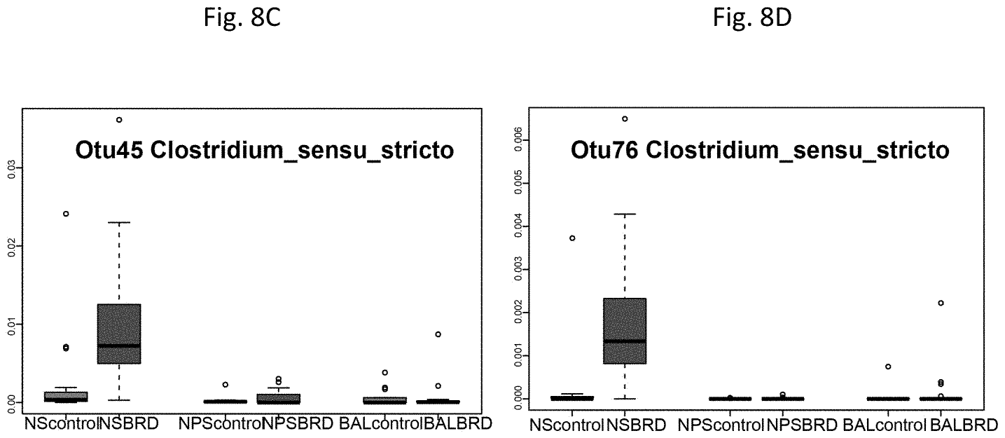

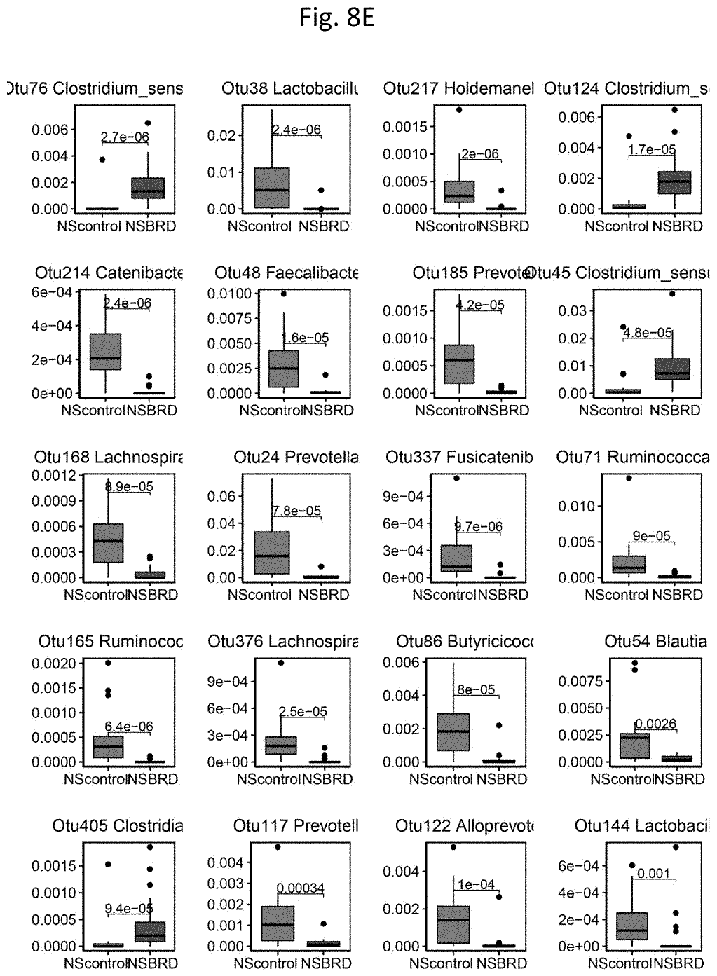

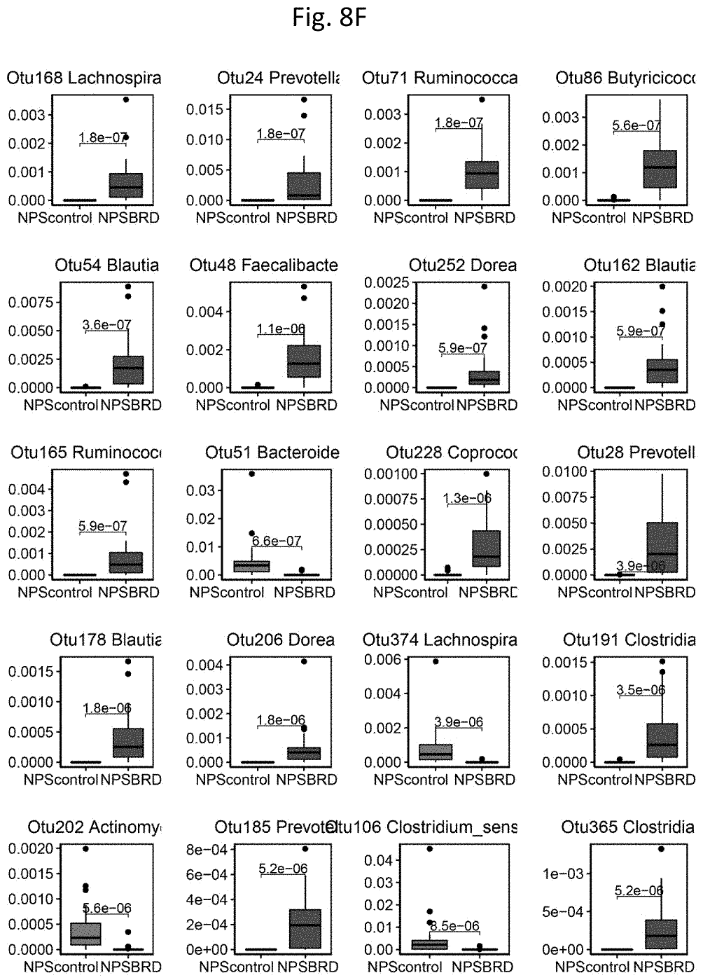

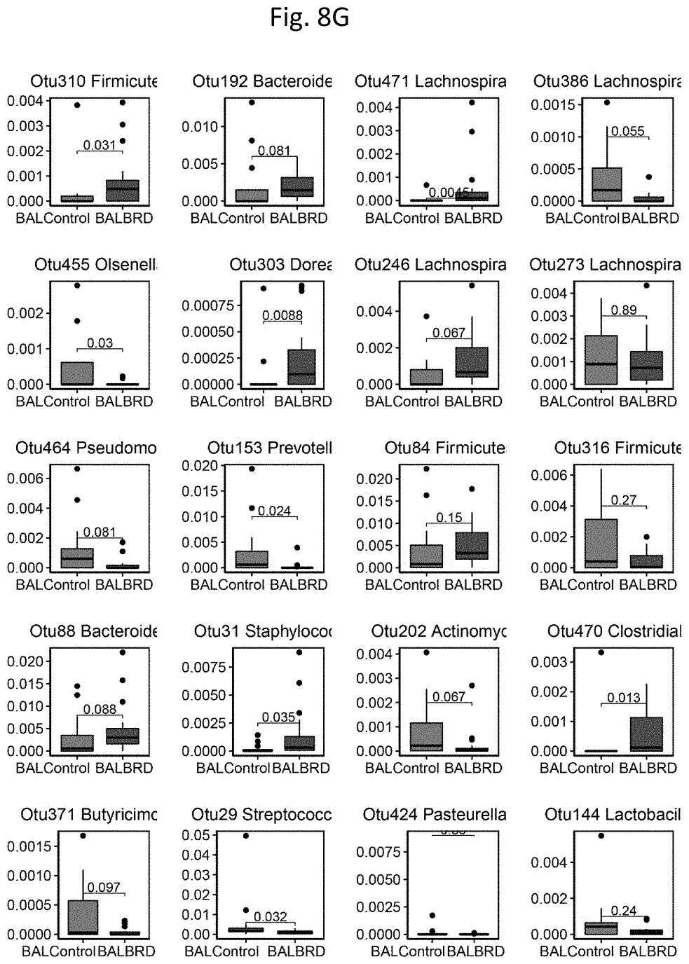

Microbiome features that differentiate calves with BRD from healthy controls were also identified by AUC-RF (FIG. 7). The highest AUCs obtained by the random forest models were 0.972, 0.961 and 0.948 using NS, NPS and BAL samples, respectively (FIG. 8A). In the NS samples, the top 20 features included many GI-tract associated OTUs, such as OTU76 (Clostridium_sensu_stricto), OTU38 (Lactobacillus), OTU48 (Faecalibacterium) and OTU71 (Ruminococcaceae). Most of these OTUs were more abundant in the healthy control calves as compared to BRD calves (FIG. 8D-8E). Interestingly, many of these OTUs were also among the top 50 features identified in the NPS and BAL samples. OTU144 (Lactobacillus) and Otu386 (Lachnospiraceae) were consistently more abundant in the three microbiome niches of healthy controls than those of calves with BRD. On the contrary, Clostridium_sensu_stricto (Otu45 and OTU76) was more abundant in all three niches of calves with BRD. However, many of the identified signatures were niche specific (FIG. 8E-8G, Tables 5-7), and some signatures even had the opposite abundance distribution between healthy and BRD calves in NS samples as they did in NPS samples.

TABLE 5
OTUs for diagnosis of BRD from NS samples

| | Source | |
|---|---|---|
| Name | strain | Partial 16S rRNA sequence |
| SEQ ID | Clostridium | TACGTATGTCGCAAGCGTTATCCGGATTTATTGGGCGTA |
| NO: 46 | butyricum | AAGCGCGTCTAGGCGGTTTGGTAAGTCTGATGTGAAAA |
| (OTU76) | strain JCM | TGCGGGGCTCAACTCCGTATTGCGTTGGAAACTGCCAA |
| | 1391 | ACTAGAGTACTGGAGAGGTGGGCGGAACTACAAGTGTA |
| | | GAGGTGAAATTCGTAGATATTTGTAGGAATGCCGATGG |
| | | GGAAGCCAGCCCACTGGACAGATACTGACGCTAAAGCG |
| | | CGAAAGCGTGGGTAGCAAACAGG |
| SEQ ID | Lactobacillus | TACGTAGGTGGCAAGCGTTGTCCGGATTTATTGGGCGT |
| NO: 47 | gasseri | AAAGCGAGTGCAGGCGGTTCAATAAGTCTGATGTGAAA |
| (OTU38) | ATCC | GCCTTCGGCTCAACCGGAGAATTGCATCAGAAACTGTT |
| | 33323 = | GAACTTGAGTGCAGAAGAGGAGAGTGGAACTCCATGTG |
| | JCM 1131 | TAGCGGTGGAATGCGTAGATATATGGAAGAACACCAGT |
| | | GGCGAAGGCGGCTCTCTGGTCTGCAACTGACGCTGAGG |
| | | CTCGAAAGCATGGGTAGCGAACAGG |
| SEQ ID | Holdemanella | TACGTAGGTGGCGAGCGTTATCCGGAATGATTGGGCGT |
| NO: 25 | biformis | AAAGGGTGCGTAGGTGGCAGATCAAGTCTGGAGTAAAA |
| (OTU217) | strain DSM | GGTATGGGCTCAACCCGTACTGGCTCTGGAAACTGATC |
| | 3989 | AGCTAGAGAACAGAAGAGGACGGCGGAACTCCATGTG |
| | | TAGCGGTAAAATGCGTAGATATATGGAAGAACACCGGT |
| | | GGCGAAGGCGGCCGTCTGGTCTGGATTCTGACACTGAA |
| | | GCACGAAAGCGTGGGGAGCAAATAGG |
| SEQ ID | Clostridium | TACGTAGGTGGCGAGCGTTGTCCGGATTTACTGGGCGT |
| NO: 48 | saudiense | AAAGGGAGCGTAGGCGGACTTTTAAGTGAGATGTGAAA |
| (OTU124) | strain JCC | TACCCGGGCTCAACTTGGGTGCTGCATTTCAAACTGGA |
| | | AGTCTAGAGTGCAGGAGAGGAGAATGGAATTCCTAGTG |
| | | TAGCGGTGAAATGCGTAGAGATTAGGAAGAACACCAGT |
| | | GGCGAAGGCGATTCTCTGGACTGTAACTGACGCTGAGG |
| | | CTCGAAAGCGTGGGGAGCAAACAGG |
| SEQ ID | Cateni- | TACGTAGGTGGCGAGCGTTATCCGGAATCATTGGGCGT |
| NO: 18 | bacterium | AAAGAGGGAGCAGGCGGCCGCAAGGGTCTGTGGTGAA |
| (OTU214) | mitsuokai | AGACCGAAGCTAAACTTCGGTGAGCCATGGAAACCGGG |
| | strain DSM | CGGCTAGAGTGCGGAAGAGGATCGTGGAATTCCATGTG |
| | 15897 | TAGCGGTGAAATGCGTAGATATATGGAGGAACACCAGT |
| | | GGCGAAGGCGACGGTCTGGGCCGCAACTGACGCTCATT |
| | | CCCGAAAGCGTGGGGAGCAAATAGG |
| SEQ ID | Faecali- | AACGTAGGTCACAAGCGTTGTCCGGAATTACTGGGTGT |
| NO: 49 | bacterium | AAAGGGAGCGCAGGCGGGAAGACAAGTTGGAAGTGAA |
| (OTU48) | prausnitzii | ATCCATGGGCTCAACCCATGAACTGCTTTCAAAACTGTT |
| | strain | TTTCTTGAGTAGTGCAGAGGTAGGCGGAATTCCCGGTG |
| | ATCC | TAGCGGTGGAATGCGTAGATATCGGGAGGAACACCAGT |
| | 27768 | GGCGAAGGCGGCCTACTGGGCACCAACTGACGCTGAGG |
| | | CTCGAAAGTGTGGGTAGCAAACAGG |
| SEQ ID | Prevotella | TACGGAAGGTCCGGGCGTTATCCGGATTTATTGGGTTTA |
| NO: 2 | stercorea | AAGGGAGCGTAGGCCGTCTGTTAAGCGTGTTGTGAAAT |
| (OTU185) | DSM 18206 | GTCGGGGCTCAACCTGGGCATTGCAGCGCGAACTGGCA |
| | strain CB35 | GACTTGAGTGCGCGGGAAGTAGGCGGAATTCGTCGTGT |
| | | AGCGGTGAAATGCTTAGATATGACGAAGAACTCCGATT |
| | | GCGAAGGCAGCCTGCTGTAGTGCAACTGACGCTGAAGC |
| | | TCGAAAGCGTGGGTATCGAACAGG |
| SEQ ID | Clostridium | TACGTAGGTGGCGAGCGTTGTCCGGATTTACTGGGCGT |
| NO: 5 | saudiense | AAAGGGAGCGTAGGCGGATTTTTAAGTGAGATGTGAAA |
| (OTU45) | strain JCC | TACTCGGGCTTAACCTGAGTGCTGCATTTCAAACTGGAA |
| | | GTCTAGAGTGCAGGAGAGGAGAAGGGAATTCCTAGTGT |
| | | AGCGGTGAAATGCGTAGAGATTAGGAAGAACACCAGT |
| | | GGCGAAGGCGCTTCTCTGGACTGTAACTGACGCTGAGG |
| | | CTCGAAAGCGTGGGGAGCAAACAGG |
| SEQ ID | Ruminococcus | TACGTAGGGGGCAAGCGTTATCCGGATTTACTGGGTGT |
| NO: 50 | faecis | AAAGGGAGCGTAGACGGAATGGCAAGTCTGATGTGAA |
| (OTU168) | JCM 15917 | AGGCCGGGGCTCAACCCCGGGACTGCATTGGAAACTGT |
| | strain Eg2 | CAATCTAGAGTACCGGAGGGGTAAGTGGAATTCCTAGT |
| | | GTAGCGGTGAAATGCGTAGATATTAGGAGGAACACCAG |
| | | TGGCGAAGGCGGCTTACTGGACGGTAACTGACGTTGAG |
| | | GCTCGAAAGCGTGGGGAGCAAACAGG |
| SEQ ID | Prevotella | TACGGAAGGTCCGGGCGTTATCCGGATTTATTGGGTTTA |
| NO: 11 | copri DSM | AAGGGAGCGTAGGCCGGAGATTAAGCGTGTTGTGAAAT |
| (OTU24) | 18205 strain | GTAGTGGCTCAACCTCTGCACTGCAGCGCGAACTGGTC |
| | JCM 13464 | TTCTTGAGTACGCACAACGTGGGCGGAATTCGTGGTGT |
| | | AGCGGTGAAATGCTTAGATATCACGAAGAACTCCGATT |
| | | GCGAAGGCAGCTCACGGGAGCGCAACTGACGCTGAAG |
| | | CTCGAAAGTGCGGGTATCGAACAGG |
| SEQ ID | Fusicateni- | TACGTAGGGGGCAAGCGTTATCCGGATTTACTGGGTGT |
| NO: 51 | bacter | AAAGGGAGCGTAGACGGCAAGGCAAGTCTGATGTGAA |
| (OTU337) | saccharivorans | AACCCAGGGCTTAACCCTGGGACTGCATTGGAAACTGT |
| | strain | CTGGCTCGAGTGCCGGAGAGGTAAGCGGAATTCCTAGT |
| | HT03-11 | GTAGCGGTGAAATGCGTAGATATTAGGAAGAACACCAG |
| | | TGGCGAAGGCGGCTTACTGGACGGTAACTGACGTTGAG |
| | | GCTCGAAAGCGTGGGGAGCAAACAGG |
| SEQ ID | Gemmiger | AACGTAGGGTGCAAGCGTTGTCCGGAATTACTGGGTGT |
| NO: 10 | formicilis | AAAGGGAGCGCAGGCGGACCGGCAAGTTGGAAGTGAA |
| (OTU71) | strain X2-56 | AACCATGGGCTCAACCCATAAATTGCTTTCAAAACTGCT |
| | | GGCCTTGAGTAGTGCAGAGGTAGGTGGAATTCCCGGTG |
| | | TAGCGGTGGAATGCGTAGATATCGGGAGGAACACCAGT |
| | | GGCGAAGGCGACCTACTGGGCACCAACTGACGCTGAGG |
| | | CTCGAAAGCATGGGTAGCAAACAGG |
| SEQ ID | Ruminococcus | TACGTATGGTGCAAGCGTTATCCGGATTTACTGGGTGTA |
| NO: 52 | faecis | AAGGGAGCGTAGACGGAGTGGCAAGTCTGATGTGAAA |
| (OTU165) | JCM 15917 | ACCCGGGGCTCAACCCCGGGACTGCATTGGAAACTGTC |
| | strain Eg2 | AATCTAGAGTACCGGAGAGGTAAGCGGAATTCCTAGTG |
| | | TAGCGGTGAAATGCGTAGATATTAGGAGGAACACCAGT |
| | | GGCGAAGGCGGCTTACTGGACGGTAACTGACGTTGAGG |
| | | CTCGAAAGCGTGGGGAGCAAACAGG |
| SEQ ID | [Eubacterium] | TACGTATGGTGCAAGCGTTATCCGGATTTACTGGGTGTA |
| NO: 53 | eligens | AAGGGAGCGTAGGTGGCAAGGCAAGCCAGAAGTGAAA |
| (OTU376) | ATCC 27750 | ACCCGGGGCTCAACCGCGGGATTGCTTTTGGAACTGTC |
| | | ATGCTGGAGTGCAGGAGGGGTGAGCGGAATTCCTAGTG |
| | | TAGCGGTGAAATGCGTAGATATTAGGAGGAACACCGGA |
| | | GGCGAAGGCGGCTCACTGGACTGTAACTGACACTGAGG |
| | | CTCGAAAGCGTGGGGAGCAAACAGG |
| SEQ ID | Butyricicoccus | TACGTAGGGAGCAAGCGTTATCCGGATTTACTGGGTGT |
| NO: 54 | pullicaecorum | AAAGGGCGAGTAGGCGGATTGGCAAGTTGGGAGTGAA |
| (OTU86) | strain 25-3 | ATGTCGGGGCTTAACCCCGGAACTGCTTCCAAAACTGTT |
| | | GATCTTGAGTGATGGAGAGGCAGGCGGAATTCCCAGTG |
| | | TAGCGGTGAAATGCGTAGATATTGGGAGGAACACCAGT |
| | | GGCGAAGGCGGCCTGCTGGACATTAACTGACGCTGAGG |
| | | AGCGAAAGCGTGGGGAGCAAACAGG |
| SEQ ID | Blautia | TACGTAGGGGGCAAGCGTTATCCGGATTTACTGGGTGT |
| NO: 55 | wexlerae | AAAGGGAGCGTAGACGGTGTGGCAAGTCTGATGTGAAA |
| (OTU54) | DSM | GGCATGGGCTCAACCTGTGGACTGCATTGGAAACTGTC |
| | 19850 | ATACTTGAGTGCCGGAGGGGTAAGCGGAATTCCTAGTG |
| | | TAGCGGTGAAATGCGTAGATATTAGGAGGAACACCAGT |
| | | GGCGAAGGCGGCTTACTGGACGGTAACTGACGTTGAGG |
| | | CTCGAAAGCGTGGGGAGCAAACAGG |
| SEQ ID | Rumini- | TACGTAGGTGGCGAGCGTTATCCGGATTTACTGGGTGT |
| NO: 56 | clostridium | AAAGGGCGCGTAGGCGGGAATGCAAGTCAGATGTGAA |
| (OTU405) | cellobioparum | ATCCAAGGGCTCAACCCTTGAACTGCATTTGAAACTGT |
| | DSM | ATTTCTTGAGTGTCGGAGAGGTTGACGGAATTCCTAGTG |
| | 1351 = | TAGCGGTGAAATGCGTAGATATTAGGAGGAACACCAGT |
| | ATCC | GGCGAAGGCGGTCAACTGGACGATAACTGACGCTGAGG |
| | 15832 strain | CGCGAAAGCGTGGGGAGCAAACAGG |
| | JCM 1422 | |
| SEQ ID | Massiliprev | TACGGAAGGTCCGGGCGTTATCCGGATTTATTGGGTTTA |
| NO: 57 | otella | AAGGGAGCGCAGGCCGCCGGGCAAGCGTGTTGTGAAAT |
| (OTU117) | massiliensis | GCAGTCGCTCAACGTCTGCACTGCAGCGCGAACTGCCC |
| | strain | AGCTTGAGTGCGCGCAACGTTGGCGGAATTCGCCGTGT |
| | Marseille- | AGCGGTGAAATGCTTAGATATGGCGAAGAACTCCGATT |
| | P2439 | GCGAAGGCAGCTGACGGGTGCGTAACTGACGCTCATGC |
| | | TCGAAAGTGCGGGTATCGAACAGG |
| SEQ ID | Prevotella- | TACGGAAGGTCCGGGCGTTATCCGGATTTATTGGGTTTA |
| NO: 58 | massilia | AAGGGAGCGTAGGCGGGCTATTAAGTCAGCTGTGAAAT |
| (OTU122) | timonensis | CCGGCGGCTCAACCGTCGGCCTGCAGTTGATACTGGTG |
| | strain | GCCTTGAGTGCGCGCAGGGGCGTTGGAATTCATGGTGT |
| | Marseille- | AGCGGTGAAATGCTCAGATATCATGAAGAACTCCGATC |
| | P2831 | GCGAAGGCAGATGCCCGGAGCGCAACTGACGCTGAGG |
| | | CTCGAAAGTGCGGGTATCAAACAGG |
| SEQ ID | Lactobacillus | TACGTAGGTGGCAAGCGTTATCCGGATTTATTGGGCGT |
| NO: 9 | mucosae | AAAGCGAGCGCAGGCGGTTTGATAAGTCTGATGTGAAA |
| (OTU144) | strain S32 | GCCTTTGGCTTAACCAAAGAAGTGCATCGGAAACTGTC |
| | | AGACTTGAGTGCAGAAGAGGACAGTGGAACTCCATGTG |
| | | TAGCGGTGGAATGCGTAGATATATGGAAGAACACCAGT |
| | | GGCGAAGGCGGCTGTCTGGTCTGCAACTGACGCTGAGG |
| | | CTCGAAAGCATGGGTAGCGAACAGG |

TABLE 6
OTUs for diagnosis of BRD from NPS samples

| | Source | |
|---|---|---|
| Name | strain | Partial 16S rRNA sequence |
| SEQ ID | Ruminococcus | TACGTAGGGGGCAAGCGTTATCCGGATTTACTGGGTGT |
| NO: 50 | faecis | AAAGGGAGCGTAGACGGAATGGCAAGTCTGATGTGAA |
| (OTU168) | JCM 15917 | AGGCCGGGGCTCAACCCCGGGACTGCATTGGAAACTGT |
| | strain Eg2 | CAATCTAGAGTACCGGAGGGGTAAGTGGAATTCCTAGT |
| | | GTAGCGGTGAAATGCGTAGATATTAGGAGGAACACCAG |
| | | TGGCGAAGGCGGCTTACTGGACGGTAACTGACGTTGAG |
| | | GCTCGAAAGCGTGGGGAGCAAACAGG |
| SEQ ID | Prevotella | TACGGAAGGTCCGGGCGTTATCCGGATTTATTGGGTTTA |
| NO: 11 | copri DSM | AAGGGAGCGTAGGCCGGAGATTAAGCGTGTTGTGAAAT |
| (OTU24) | 18205 strain | GTAGTGGCTCAACCTCTGCACTGCAGCGCGAACTGGTC |
| | JCM 13464 | TTCTTGAGTACGCACAACGTGGGCGGAATTCGTGGTGT |
| | | AGCGGTGAAATGCTTAGATATCACGAAGAACTCCGATT |
| | | GCGAAGGCAGCTCACGGGAGCGCAACTGACGCTGAAG |
| | | CTCGAAAGTGCGGGTATCGAACAGG |
| SEQ ID | Gemmiger | AACGTAGGGTGCAAGCGTTGTCCGGAATTACTGGGTGT |
| NO: 10 | formicilis | AAAGGGAGCGCAGGCGGACCGGCAAGTTGGAAGTGAA |
| (OTU71) | strain X2-56 | AACCATGGGCTCAACCCATAAATTGCTTTCAAAACTGCT |
| | | GGCCTTGAGTAGTGCAGAGGTAGGTGGAATTCCCGGTG |
| | | TAGCGGTGGAATGCGTAGATATCGGGAGGAACACCAGT |
| | | GGCGAAGGCGACCTACTGGGCACCAACTGACGCTGAGG |
| | | CTCGAAAGCATGGGTAGCAAACAGG |
| SEQ ID | Butyricicoccus | TACGTAGGGAGCAAGCGTTATCCGGATTTACTGGGTGT |
| NO: 54 | pullicaecorum | AAAGGGCGAGTAGGCGGATTGGCAAGTTGGGAGTGAA |
| (OTU86) | strain 25-3 | ATGTCGGGGCTTAACCCCGGAACTGCTTCCAAAACTGTT |
| | | GATCTTGAGTGATGGAGAGGCAGGCGGAATTCCCAGTG |
| | | TAGCGGTGAAATGCGTAGATATTGGGAGGAACACCAGT |
| | | GGCGAAGGCGGCCTGCTGGACATTAACTGACGCTGAGG |
| | | AGCGAAAGCGTGGGGAGCAAACAGG |
| SEQ ID | Blautia | TACGTAGGGGGCAAGCGTTATCCGGATTTACTGGGTGT |
| NO: 55 | wexlerae | AAAGGGAGCGTAGACGGTGTGGCAAGTCTGATGTGAAA |
| (OTU54) | DSM | GGCATGGGCTCAACCTGTGGACTGCATTGGAAACTGTC |
| | 19850 | ATACTTGAGTGCCGGAGGGGTAAGCGGAATTCCTAGTG |
| | | TAGCGGTGAAATGCGTAGATATTAGGAGGAACACCAGT |
| | | GGCGAAGGCGGCTTACTGGACGGTAACTGACGTTGAGG |
| | | CTCGAAAGCGTGGGGAGCAAACAGG |
| SEQ ID | Faecali- | AACGTAGGTCACAAGCGTTGTCCGGAATTACTGGGTGT |
| NO: 49 | bacterium | AAAGGGAGCGCAGGCGGGAAGACAAGTTGGAAGTGAA |
| (OTU48) | prausnitzii | ATCCATGGGCTCAACCCATGAACTGCTTTCAAAACTGTT |
| | strain | TTTCTTGAGTAGTGCAGAGGTAGGCGGAATTCCCGGTG |
| | ATCC | TAGCGGTGGAATGCGTAGATATCGGGAGGAACACCAGT |
| | 27768 | GGCGAAGGCGGCCTACTGGGCACCAACTGACGCTGAGG |
| | | CTCGAAAGTGTGGGTAGCAAACAGG |
| SEQ ID | Dorea | TACGTAGGGGGCAAGCGTTATCCGGATTTACTGGGTGT |
| NO: 59 | formicigenerans | AAAGGGAGCGTAGACGGCTGTGCAAGTCTGAAGTGAA |
| (OTU252) | strain | AGGCATGGGCTCAACCTGTGGACTGCTTTGGAAACTGT |
| | ATCC | GCAGCTAGAGTGTCGGAGAGGTAAGTGGAATTCCTAGT |
| | 27755 | GTAGCGGTGAAATGCGTAGATATTAGGAGGAACACCAG |
| | | TGGCGAAGGCGGCTTACTGGACGATGACTGACGTTGAG |
| | | GCTCGAAAGCGTGGGGAGCAAACAGG |
| SEQ ID | Blautia | TACGTAGGGGGCAAGCGTTATCCGGATTTACTGGGTGT |
| NO: 14 | obeum | AAAGGGAGCGTAGACGGACTGGCAAGTCTGATGTGAA |
| (OTU162) | ATCC | AGGCGGGGGCTCAACCCCTGGACTGCATTGGAAACTGT |
| | 29174 | TAGTCTTGAGTGCCGGAGAGGTAAGCGGAATTCCTAGT |
| | | GTAGCGGTGAAATGCGTAGATATTAGGAGGAACACCAG |
| | | TGGCGAAGGCGGCTTACTGGACGGTAACTGACGTTGAG |
| | | GCTCGAAAGCGTGGGGAGCAAACAGG |
| SEQ ID | Ruminococcus | TACGTATGGTGCAAGCGTTATCCGGATTTACTGGGTGTA |
| NO: 52 | faecis | AAGGGAGCGTAGACGGAGTGGCAAGTCTGATGTGAAA |
| (OTU165) | JCM 15917 | ACCCGGGGCTCAACCCCGGGACTGCATTGGAAACTGTC |
| | strain Eg2 | AATCTAGAGTACCGGAGAGGTAAGCGGAATTCCTAGTG |
| | | TAGCGGTGAAATGCGTAGATATTAGGAGGAACACCAGT |
| | | GGCGAAGGCGGCTTACTGGACGGTAACTGACGTTGAGG |
| | | CTCGAAAGCGTGGGGAGCAAACAGG |
| SEQ ID | Bacteroides | TACGGAGGATCCGAGCGTTATCCGGATTTATTGGGTTTA |
| NO: 60 | fragilis | AAGGGAGCGTAGGTGGACTGGTAAGTCAGTTGTGAAAG |
| (OTU51) | strain | TTTGCGGCTCAACCGTAAAATTGCAGTTGATACTGTCAG |
| | NCTC 9343 | TCTTGAGTACAGTAGAGGTGGGCGGAATTCGTGGTGTA |
| | | GCGGTGAAATGCTTAGATATCACGAAGAACTCCGATTG |
| | | CGAAGGCAGCTCACTGGACTGCAACTGACACTGATGCT |
| | | CGAAAGTGTGGGTATCAAACAGG |
| SEQ ID | Coprococcus | TACGTATGGTGCAAGCGTTATCCGGATTTACTGGGTGTA |
| NO: 61 | comes | AAGGGAGCGTAGACGGCTGTGTAAGTCTGAAGTGAAAG |
| (OTU228) | ATCC | CCCGGGGCTCAACCCCGGGACTGCTTTGGAAACTATGC |
| | 27758 | AGCTAGAGTGTCGGAGAGGTAAGTGGAATTCCCAGTGT |
| | | AGCGGTGAAATGCGTAGATATTGGGAGGAACACCAGTG |
| | | GCGAAGGCGGCTTACTGGACGATGACTGACGTTGAGGC |
| | | TCGAAAGCGTGGGGAGCAAACAGG |
| SEQ ID | Prevotella | TACGGAAGGTCCGGGCGTTATCCGGATTTATTGGGTTTA |
| NO: 62 | copri DSM | AAGGGAGCGTAGGCCGGAGATTAAGCGTGTTGTGAAAT |
| (OTU28) | 18205 strain | GTAGATGCTCAACGTCTGCACTGCAGCGCGAACTGGTT |
| | JCM 13464 | TCCTTGAGTACGCACAAAGTGGGCGGAATTCGTGGTGT |
| | | AGCGGTGAAATGCTTAGATATCACGAAGAACTCCGATT |
| | | GCGAAGGCAGCTCACTGGAGCGCAACTGACGCTGAAGC |
| | | TCGAAAGTGCGGGTATCGAACAGG |
| SEQ ID | Blautia luti | TACGTAGGGGGCAAGCGTTATCCGGATTTACTGGGTGT |
| NO: 63 | strain BInIX | AAAGGGAGCGTAGACGGATGGACAAGTCTGATGTGAA |
| (OTU178) | | AGGCTGGGGCTCAACCCCGGGACTGCATTGGAAACTGC |
| | | CCGTCTTGAGTGCCGGAGAGGTAAGCGGAATTCCTAGT |
| | | GTAGCGGTGAAATGCGTAGATATTAGGAGGAACACCAG |
| | | TGGCGAAGGCGGCTTACTGGACGGTAACTGACGTTGAG |
| | | GCTCGAAAGCGTGGGGAGCAAACAGG |
| SEQ ID | Dorea | TACGTAGGGGGCAAGCGTTATCCGGATTTACTGGGTGT |
| NO: 64 | longicatena | AAAGGGAGCGTAGACGGCACGGCAAGCCAGATGTGAA |
| (OTU206) | strain 111- | AGCCCGGGGCTCAACCCCGGGACTGCATTTGGAACTGC |
| | 35 | TGAGCTAGAGTGTCGGAGAGGCAAGTGGAATTCCTAGT |
| | | GTAGCGGTGAAATGCGTAGATATTAGGAGGAACACCAG |
| | | TGGCGAAGGCGGCTTGCTGGACGATGACTGACGTTGAG |
| | | GCTCGAAAGCGTGGGGAGCAAACAGG |
| SEQ ID | [Ruminococcus] | TACGTAGGGGGCAAGCGTTATCCGGATTTACTGGGTGT |
| NO: 65 | gnavus | AAAGGGAGCGTAGACGGCATGGCAAGCCAGATGTGAA |
| (OTU374) | ATCC | AGCCCGGGGCTCAACCCCGGGACTGCATTTGGAACTGT |
| | 29149 | CAGGCTAGAGTGTCGGAGAGGAAAGCGGAATTCCTAGT |
| | | GTAGCGGTGAAATGCGTAGATATTAGGAGGAACACCAG |
| | | TGGCGAAGGCGGCTTTCTGGACGATGACTGACGTTGAG |
| | | GCTCGAAAGCGTGGGGAGCAAACAGG |
| SEQ ID | [Eubacterium] | TACGTAGGGAGCAAGCGTTATCCGGATTTACTGGGTGT |
| NO: 66 | hallii | AAAGGGTGCGTAGGTGGCAGTGCAAGTCAGATGTGAAA |
| (OTU191) | strain | GGCCGGGGCTCAACCCCGGAGCTGCATTTGAAACTGCA |
| | ATCC | TAGCTAGAGTACAGGAGAGGCAGGCGGAATTCCTAGTG |
| | 27751 | TAGCGGTGAAATGCGTAGATATTAGGAGGAACACCAGT |
| | | GGCGAAGGCGGCCTGCTGGACTGTTACTGACACTGAGG |
| | | CACGAAAGCGTGGGGAGCAAACAGG |
| SEQ ID | Schaalia | TACGTAGGGCGCGAGCGTTGTCCGGAATTATTGGGCGT |
| NO: 67 | cardiffensis | AAAGGGCTTGTAGGCGGTTGGTTGCGTCTGCCGTGAAA |
| (OTU202) | strain | TCCTCTGGCTTAACTGGGGGCGTGCGGTGGGTACGGGC |
| | CCUG | TGGCTTGAGTGCGGTAGGGGAGACTGGAATTCCTGGTG |
| | 44997 | TAGCGGTGGAATGCGCAGATATCAGGAGGAACACCGGT |
| | | GGCGAAGGCGGGTCTCTGGGCCGTTACTGACGCTGAGG |
| | | AGCGAAAGCGTGGGGAGCGAACAGG |
| SEQ ID | Prevotella | TACGGAAGGTCCGGGCGTTATCCGGATTTATTGGGTTTA |
| NO: 2 | stercorea | AAGGGAGCGTAGGCCGTCTGTTAAGCGTGTTGTGAAAT |
| (OTU185) | DSM 18206 | GTCGGGGCTCAACCTGGGCATTGCAGCGCGAACTGGCA |
| | strain CB35 | GACTTGAGTGCGCGGGAAGTAGGCGGAATTCGTCGTGT |
| | | AGCGGTGAAATGCTTAGATATGACGAAGAACTCCGATT |
| | | GCGAAGGCAGCCTGCTGTAGTGCAACTGACGCTGAAGC |
| | | TCGAAAGCGTGGGTATCGAACAGG |
| SEQ ID | Clostridium | TACGTAGGTGGCGAGCGTTATCCGGATTTACTGGGCGT |
| NO: 68 | perfringens | AAAGGGAGCGTAGGCGGATGATTAAGTGGGATGTGAA |
| (OTU106) | ATCC | ATACCCGGGCTCAACTTGGGTGCTGCATTCCAAACTGGT |
| | 13124 | TATCTAGAGTGCAGGAGAGGAGAGTGGAATTCCTAGTG |
| | | TAGCGGTGAAATGCGTAGAGATTAGGAAGAACACCAGT |
| | | GGCGAAGGCGACTCTCTGGACTGTAACTGACGCTGAGG |
| | | CTCGAAAGCGTGGGGAGCAAACAGG |
| SEQ ID | [Ruminococcus] | TACGTAGGGGGCAAGCGTTATCCGGATTTACTGGGTGT |
| NO: 69 | gnavus | AAAGGGAGCGTAGACGGCATGGCAAGCCAGATGTGAA |
| (OTU365) | ATCC | AGCCCGGGGCTCAACCCCGGGACTGCATTTGGAACTGT |
| | 29149 | CAGGCTAGAGTGTCGGAGAGGAAAGCGGAATTCCTAGT |
| | | GTAGCGGTGAAATGCGTAGATATTAGGAGGAACACCAG |
| | | TGGCGAAGGCGGCTTTCTGGACGATGACTGACGTTGAG |
| | | GCTCGAAAGCGTGGGGAGCAAACAGG |

TABLE 7
OTUs for diagnosis of BRD from BAL samples

| | Source | |
|---|---|---|
| Name | strain | Partial 16S rRNA sequence |
| SEQ ID | Caldalkali- | TACGTAGGGGGCAAGCGTTGTCCGGAATGATTGGGCGT |
| NO: 70 | bacillus | AAAGGGCGCGTAGGCGGCCATTTAAGTCTGAAGTGAAA |
| (OTU310) | thermarum | GTCCTGCTTTCAAGGTGGGAACTGCTTTGGATACTGGAT |
| | strain HA6 | GGCTTGAGTGCAGGAGAGGTAAGCGGAATTCCCGGTGT |
| | | AGCGGTGAAATGCGTAGAGATCGGGAGGAACACCAGT |
| | | GGCGAAGGCGGCTTACTGGACTGTAACTGACGCTGAGG |
| | | CGCGAAAGTGTGGGGAGCAAACAGG |
| SEQ ID | Solitalea | TACGGAGGACCCGAGCGTTATCCGGATTTATTGGGTTTA |
| NO: 71 | canadensis | AAGGGTGCGTAGGCGGGCGATTAAGTCAGAGGTGAAA |
| (OTU192) | DSM 3403 | GCCCGGGGCTCAACTCCGGAACTGCCTTTGATACTGGTT |
| | | GTCTTGAGTCATGACGAGGATGGCGGAATGTGATGTGT |
| | | AGCGGTGAAATGCATAGATATGTCACAGAACACCGATT |
| | | GCGAAGGCAGCTGTCTAGAGATGAACTGACGCTGAGGC |
| | | ACGAAAGTATGGGGATCAAACAGG |
| SEQ ID | Anaerostipes | TACGTATGGTGCAAGCGTTATCCGGATTTACTGGGTGTA |
| NO: 72 | caccae | AAGGGTGCGTAGGTGGTATGGTAAGTCAGAAGTGAAAA |
| (OTU471) | strain L1-92 | CACGGGGCTCAACCCCGTGACTGCTTTTGAAACTATCA |
| | | AACTAGAGTCCAGGAGAGGTAAGCGGAATTCCTAGTGT |
| | | AGCGGTGAAATGCGTAGATATTAGGAGGAACACCAGTG |
| | | GCGAAGGCGGCTTACTGGACTGGTACTGACACTGAGGC |
| | | ACGAAAGCGTGGGGAGCAAACAGG |
| SEQ ID | Eisenbergiella | TACGTAGGGGGCAAGCGTTATCCGGATTTACTGGGTGT |
| NO: 73 | massiliensis | AAAGGGAGCGTAGACGGCATGACAAGCCAGATGTGAA |
| (OTU386) | strain AT11 | AACCCAGGGCTCAACCCTGGGACTGCATTTGGAACTGC |
| | | CAGGCTGGAGTGCAGGAGAGGTAAGCGGAATTCCTAGT |
| | | GTAGCGGTGAAATGCGTAGATATTAGGAGGAACACCAG |
| | | TGGCGAAGGCGGCTTACTGGACTGTAACTGACGTTGAG |
| | | GCTCGAAAGCGTGGGGAGCAAACAGG |
| SEQ ID | Olsenella | TACGTAGGGGGCAAGCGTTATCCGGATTCATTGGGCGT |
| NO: 74 | profusa | AAAGCGCGCGTAGGCGGCCAGCCTGGTCGGGAGTCAAA |
| (OTU455) | DSM 13989 | TCCGGGGGCTCAACCCCCGTCCGCTCCCGATACCGGCC |
| | | GGCTTGAGTCTGGCAGGGGAAGGCGGAATTCCCGGTGT |
| | | AGCGGTGGAATGCGCAGATATCGGGAAGAACACCGGT |
| | | GGCGAAGGCGGCCTTCTGGGCCACGACTGACGCTGAGG |
| | | CGCGAAAGCTAGGGGAGCGAACAGG |
| SEQ ID | Dorea | TACGTAGGGGGCAAGCGTTATCCGGATTTACTGGGTGT |
| NO: 75 | formicigenerans | AAAGGGAGCGTAGACGGCTATGCAAGTCTGAAGTGAA |
| (OTU303) | strain | AGGCATGGGCTCAACCTGTGGACTGCTTTGGAAACTGT |
| | ATCC | GAAGCTAGAATGTCGGAGAGGCAAGGGGAATTCCTAGT |
| | 27755 | GTAGCGGTGAAATGCGTAGATATTAGGAGGAACACCAG |
| | | TGGCGAAGGCGCCTTGCTGGACGATGATTGACGTTGAG |
| | | GCTCGAAAGCGTGGGGAGCAAACAGG |
| SEQ ID | Blautia | TACGTAGGGGGCAAGCGTTATCCGGATTTACTGGGTGT |
| NO: 76 | wexlerae | AAAGGGAGCGTAGACGGAAGGTCAAGTCAGGTGTGAA |
| (OTU246) | DSM 19850 | AGGCATGGGCTCAACCCGTGGACTGCACTTGAAACTGG |
| | | TCATCTCGAGTGTCGGAGGGGCAGGCGGAATTCCTGGT |
| | | GTAGCGGTGAAATGCGTAGATATCAGGAAGAACACCGG |
| | | TGGCGAAGGCGGCCTGCTGGACGATCACTGACGTTGAG |
| | | GCTCGAAGGCGTGGGGAGCAAACAGG |
| SEQ ID | [Eubacterium] | TACGTATGGTGCAAGCGTTATCCGGATTTACTGGGTGTA |
| NO: 77 | rectale | AAGGGAGCGTAGGCGGCTTGGCAAGTCTGATGTGAAAA |
| (OTU273) | ATCC | CCCGGGGCTCAACCTCGGGACTGCATTGGAAACTGTCG |
| | 33656 | AGCTGGAGTGTCGGAGAGGTAAGCGGAATTCCTAGTGT |
| | | AGCGGTGAAATGCGTAGATATTAGGAGGAACACCAGTG |
| | | GCGAAGGCGGCTTACTGGACGATAACTGACGCTGAGGC |
| | | TCGAAAGCGTGGGGAGCAAACAGG |
| SEQ ID | Pseudomonas | TACAGAGGGTGCAAGCGTTAATCGGAATTACTGGGCGT |
| NO: 78 | lini strain | AAAGCGCGCGTAGGTGGTTCGTTAAGTTGGATGTGAAA |
| (OTU464) | DLE411J | TCCCCGGGCTCAACCTGGGAACTGCATTCAAAACTGAC |
| | | GAGCTAGAGTATGGTAGAGGGTGGTGGAATTTCCTGTG |
| | | TAGCGGTGAAATGCGTAGATATAGGAAGGAACACCAGT |
| | | GGCGAAGGCGACCACCTGGACTGATACTGACACTGAGG |
| | | TGCGAAAGCGTGGGGAGCAAACAGG |
| SEQ ID | Prevotella | TACGGAAGGTTCGGGCGTTATCCGGATTTATTGGGTTTA |
| NO: 79 | shahii strain | AAGGGAGCGTAGGCCGCTGTTCAAGCGTGTCGTGAAAT |
| (OTU153) | EHS11 | GCGGTTGCTCAACGATCGCACTGCGGCGCGAACTGTCC |
| | | GGCTTGAGTCAAGTGAAAGTAGGCGGAATTCGTGGTGT |
| | | AGCGGTGAAATGCTTAGATATCACGAGGAACTCCGATT |
| | | GCGAAGGCAGCTTACTGTAGTTGTACTGACGCTGAAGC |
| | | TCGAAGGTGCGGGTATCGAACAGG |
| SEQ ID | Kroppenstedtia | TACGTAGGGGGCAAGCGTTGTCCGGAATGATTGGGCGT |
| NO: 80 | pulmonis | AAAGGGCGCGTAGGCGGCCTGTTAAGTCTGGAGTGAAA |
| (OTU84) | strain | CTCCCGCTTTTAAGGTGGGAACTGCTTTGGATACTGATG |
| | W9323 | GGCTTGAGTGCAGGAGAGGTAAGCGGAATTCCCGGTGT |
| | | AGCGGTGAAATGCGTAGAGATCGGGAGGAACACCAGT |
| | | GGCGAAGGCGGCTTACTGGACTGTAACTGACGCTGAGG |
| | | CGCGAAAGTGTGGGGAGCAAACAGG |
| SEQ ID | Geosporobacter | TACGTAGGGGGCAAGCGTTGTCCGGAATGACTGGGCGT |
| NO: 81 | ferrireducens | AAAGGGCGTGTAGGCGGCCGATTAAGTCTGGAGTGAAA |
| (OTU316) | strain | GTCCACTTTTCAAGGGTGGAATTGCTTTGGATACTGGTT |
| | IRF9 | GGCTTGAGTGCGGAAGAGGTAAGTGGAATTCCCAGTGT |
| | | AGCGGTGAAATGCGTAGAGATTGGGAGGAACACCAGT |
| | | GGCGAAGGCGACTTACTGGGCCGTAACTGACGCTGAGG |
| | | CGCGAAAGCGTGGGGAGCGAACAGG |
| SEQ ID | Mediterranea | TACGGAAGATGCGAGCGTTATCCGGATTTATTGGGTTTA |
| NO: 82 | massiliensis | AAGGGAGCGTAGGCGGGTTTTTAAGTCAGCTGTGAAAG |
| (OTU88) | strain | TTTGAGGCTCAACCTTAAAATTGCAGTTGATACTGGAG |
| | Marseille- | ACCTTGAGTGCGGTAAAGGTAGGCGGAATTCGTGGTGT |
| | P2645 | AGCGGTGAAATGCTTAGATATCACGAAGAACTCCGATT |
| | | GCGAAGGCAGCTTACTGGGCCGTAACTGACGCTGATGC |
| | | TCGAAAGTGCGGGTATCAAACAGG |
| SEQ ID | Staphylococcus | TACGTAGGGGGCAAGCGTTATCCGGATTTACTGGGTGT |
| NO: 83 | aureus | AAAGGGAGCGTAGACGGCACGGCAAGCCAGATGTGAA |
| (OTU31) | strain S33 R | AGCCCGGGGCTCAACCCCGGGACTGCATTTGGAACTGC |
| | | TGAGCTAGAGTGTCGGAGAGGCAAGTGGAATTCCTAGT |
| | | GTAGCGGTGAAATGCGTAGATATTAGGAGGAACACCAG |
| | | TGGCGAAGGCGGCTTGCTGGACGATGACTGACGTTGAG |
| | | GCTCGAAAGCGTGGGGAGCAAACAGG |
| SEQ ID | Schaalia | TACGTAGGGCGCGAGCGTTGTCCGGAATTATTGGGCGT |
| NO: 67 | cardiffensis | AAAGGGCTTGTAGGCGGTTGGTTGCGTCTGCCGTGAAA |
| (OTU202) | strain | TCCTCTGGCTTAACTGGGGGCGTGCGGTGGGTACGGGC |
| | CCUG | TGGCTTGAGTGCGGTAGGGGAGACTGGAATTCCTGGTG |
| | 44997 | TAGCGGTGGAATGCGCAGATATCAGGAGGAACACCGGT |
| | | GGCGAAGGCGGGTCTCTGGGCCGTTACTGACGCTGAGG |
| | | AGCGAAAGCGTGGGGAGCGAACAGG |
| SEQ ID | Flavonifractor | TACGTAGGTGGCGAGCGTTATCCGGATTTACTGGGTGT |
| NO: 84 | plautii | AAAGGGTGTGTAGGCGGGATGGCAAGTCAGATGTGAA |
| (OTU470) | strain Prevot | ATCCGGGGGCTCAACCCCCGGCCTGCATTTGAAACTGC |
| | S1 | TGTTCTTGAGTGTCGGAGAGGTAAACGGAATTCCCAGT |
| | | GTAGCGGTGAAATGCGTAGATATTGGGAGGAACACCAG |
| | | TGGCGAAGGCGGTTTACTGGACGACAACTGACGCTGAG |
| | | ACACGAAAGCGTGGGGAGCAAACAGG |
| SEQ ID | Butyricimonas | TACGGGGGATGCGAGCGTTATCCGGATTTATTGGGTTTA |
| NO: 85 | virosa | AAGGGCGCGTAGGCGGGACGTCAAGTCAGCGGTAAAA |
| (OTU371) | strain MT12 | GACTGCAGCTAAACTGTAGCACGCCGTTGAAACTGGCG |
| | | CCCTGGAGACGAGACGAGGGAGGCGGAACAAGTGAAG |
| | | TAGCGGTGAAATGCATAGATATCACTTGGAACCCCGAT |
| | | AGCGAAGGCAGCTTCCCAGGCTCGTTCTGACGCTGATG |
| | | CGCGAGAGCGTGGGTAGCGAACAGG |
| SEQ ID | Streptococcus | TACGTAGGTCCCGAGCGTTGTCCGGATTTATTGGGCGTA |
| NO: 20 | pasteurianus | AAGCGAGCGCAGGCGGTTTAATAAGTCTGAAGTTAAAG |
| (OTU29) | strain CIP | GCAGTGGCTTAACCATTGTTCGCTTTGGAAACTGTTAAA |
| | 107122 | CTTGAGTGCAGAAGGGGAGAGTGGAATTCCATGTGTAG |
| | | CGGTGAAATGCGTAGATATATGGAGGAACACCGGTGGC |
| | | GAAAGCGGCTCTCTGGTCTGTAACTGACGCTGAGGCTC |
| | | GAAAGCGTGGGGAGCAAACAGG |
| SEQ ID | Haemophilus | TACGGGGGGTGCGAGCGTTAATCGGAATAACTGGGCGT |
| NO: 86 | sputorum | AAAGGGCACGCAGGCGGTGACTTAAGTGAGATGTGAA |
| (OTU424) | CCUG | AGCCCCGGGCTTAACCTGGGAATTGCATTTCATACTGG |
| | 13788 | GTCGCTAGAGTACTTTAGGGAGGGGTAGAATTCCACGT |
| | | GTAGCGGTGAAATGCGTAGAGATGTGGAGGAATACCGA |
| | | AGGCGAAGGCAGCCCCTTGGGAATGTACTGACGCTCAT |
| | | GTGCGAAAGCGTGGGGAGCAAACAGG |
| SEQ ID | Lactobacillus | TACGTAGGTGGCAAGCGTTATCCGGATTTATTGGGCGT |
| NO: 9 | mucosae | AAAGCGAGCGCAGGCGGTTTGATAAGTCTGATGTGAAA |
| (OTU144) | strain S32 | GCCTTTGGCTTAACCAAAGAAGTGCATCGGAAACTGTC |
| | | AGACTTGAGTGCAGAAGAGGACAGTGGAACTCCATGTG |
| | | TAGCGGTGGAATGCGTAGATATATGGAAGAACACCAGT |
| | | GGCGAAGGCGGCTGTCTGGTCTGCAACTGACGCTGAGG |
| | | CTCGAAAGCATGGGTAGCGAACAGG |

Some features, such as Turicibacter (OTU85), Bacteroides (OTU83 and OTU198), and Prevotella (OTU132), were detected in the lungs of healthy calves upon feedlot arrival (A9) at levels consistent with the neutral model of drift from the mid-nare region (NS), but were found to be over-represented in lungs of calves with BRD at arrival. Other species, such as Otu31 (Staphylococcus), followed similar patterns. OTUs associated with common BRD pathogens (OTU9-Mycoplasma and OTU12-Histophilus) were over-represented in the lungs of calves with BRD at arrival when NS samples were used as the source environment, but were under-represented when NPS samples were used as the source. When NPS samples were used as the source, OTUs associated with Moraxella (OTU22 and OTU646), Pseudomonas (OTU464), and Clostridium_sensu_stricto (OTU45 and OTU76) were found to be over-represented in diseased calves but were neutrally distributed in healthy calves at feedlot arrival.

When cattle were diagnosed with BRD, the pattern of microbial drift shifted from neutral towards selective. Fewer microbes predicted by the neutral model were detected in the lungs of diseased calves (NS 15%, NPS 15%), while other microbes were selectively enriched (NS 14%, NPS 13%). Signatures of BRD, such as Otu37 (Bifidobacterium), became over-represented when the calves developed BRD, using either NS or NPS samples as the source environment. Other species became over-represented in the lungs of BRD calves in a niche-specific manner: OTU38 (Lactobacillus), OTU71 (Ruminococcaceae), Otu11 (Enterobacteriaceae), Otu31 (Staphylococcus), Otu92 (Prevotella), Otu438 (Neisseria), Otu523 (Corynebacterium) and Otu913 (Corynebacterium) were over-represented in BRD lungs when NS samples were used as the source, while OTU76 (Clostridium_sensu_stricto), Otu83 (Bacteroides) and Otu340 (Streptococcus) were over-represented when NPS samples were used as the source.

### CONCLUSION

This study was the first to characterize three niches of the bovine respiratory microbiome and to track the longitudinal and cross-sectional changes that occur in these communities during the onset of BRD. Our results confirm that the three niches, sampled from the nasal cavity, nasopharynx, and lungs, have different community structures and members. Using random forest modeling, we identified sets of bacterial features from each niche that can be used to predict and diagnose BRD in cattle. Finally, we demonstrated that microbial dispersal from the upper airway to the lungs can be predicted by a neutral model, and that this dispersal process is influenced by BRD. Overall, this study demonstrates the importance of respiratory microbes in the maintenance of health, and suggests that therapies based on these microbes may be useful for the prevention and treatment of BRD.

### REFERENCES

1. Nicola I, Cerutti F, Grego E, Bertone I, Gianella P, D'Angelo A, Peletto S, Bellino C: Characterization of the upper and lower respiratory tract microbiota in Piedmontese calves. Microbiome 2017, 5:152.
2. Lima S F, Teixeira A G, Higgins C H, Lima F S, Bicalho R C: The upper respiratory tract microbiome and its potential role in bovine respiratory disease and otitis media. Sci Rep 2016, 6:29050.
3. Holman D B, McAllister T A, Topp E, Wright A-DG, Alexander T W: The nasopharyngeal microbiota of feedlot cattle that develop bovine respiratory disease. Veterinary microbiology 2015, 180:90-95.
4. Klima C L, Zaheer R, Cook S R, Booker C W, Hendrick S, Alexander T W, McAllister T A: Pathogens of bovine respiratory disease in North American feedlots conferring multidrug resistance via integrative conjugative elements. Journal of clinical microbiology 2014, 52:438-448.
5. Taylor J D, Fulton R W, Lehenbauer T W, Step D L, Confer A W: The epidemiology of bovine respiratory disease: What is the evidence for predisposing factors? The Canadian Veterinary Journal 2010, 51:1095.
6. Holman D B, Hallewell J, Alexander T W: The nasopharyngeal microbiota of beef cattle before and after transport to a feedlot. Journal of Animal Science 2017, 95:29-29.
7 Timsit E, Workentine M, Schryvers A B, Holman D B, van der Meer F, Alexander T W: Evolution of the nasopharyngeal microbiota of beef cattle from weaning to 40 days after arrival at a feedlot. Veterinary Microbiology 2016, 187:75-81.
8. Holman D B, Timsit E, Alexander T W: The nasopharyngeal microbiota of feedlot cattle. Scientific Reports 2015, 5.
9. Holman D B, McAllister T A, Topp E, Wright A D G, Alexander T W: The nasopharyngeal microbiota of feedlot cattle that develop bovine respiratory disease. Veterinary Microbiology 2015, 180:90-95.
10. Zeineldin M, Lowe J, de Godoy M, Maradiaga N, Ramirez C, Ghanem M, Abd El-Raof Y, Aldridge B: Disparity in the nasopharyngeal microbiota between healthy cattle on feed, at entry processing and with respiratory disease. Veterinary Microbiology 2017, 208:30-37.
11. Holman D B, Timsit E, Alexander T W: The nasopharyngeal microbiota of feedlot cattle. Scientific reports 2015, 5:15557.
12. Timsit E, Workentine M, van der Meer F, Alexander T: Distinct bacterial metacommunities inhabit the upper and lower respiratory tracts of healthy feedlot cattle and those diagnosed with bronchopneumonia. Veterinary Microbiology 2018.
13. Zeineldin M, Lowe J, de Godoy M, Maradiaga N, Ramirez C, Ghanem M, El-Raof Y A, Aldridge B: Disparity in the nasopharyngeal microbiota between healthy cattle on feed, at entry processing and with respiratory disease. Veterinary microbiology 2017, 208:30-37.
14. McDaneld T G, Kuehn L A, Keele J W: Evaluating the microbiome of two sampling locations in the nasal cavity of cattle with bovine respiratory disease complex (BRDC). Journal of animal science 2018, 96:1281-1287.
15. Schloss P D, Westcott S L, Ryabin T, Hall J R, Hartmann M, Hollister E B, Lesniewski R A, Oakley B B, Parks D H, Robinson C J: Introducing mothur: open-source, platform-independent, community-supported software for describing and comparing microbial communities. Applied and environmental microbiology 2009, 75:7537-7541.
16. Huse S M, Welch D M, Morrison H G, Sogin M L: Ironing out the wrinkles in the rare biosphere through improved OTU clustering. Environmental microbiology 2010, 12:1889-1898.
17. Wang Q, Garrity G M, Tiedje J M, Cole J R: Naive Bayesian classifier for rapid assignment of rRNA sequences into the new bacterial taxonomy. Applied and environmental microbiology 2007, 73:5261-5267.
18. Pragman A A, Lyu T, Baller J A, Gould T J, Kelly R F, Reilly C S, Isaacson R E, Wendt C H: The lung tissue microbiota of mild and moderate chronic obstructive pulmonary disease. Microbiome 2018, 6:7.
19. Dickson R P, Erb-Downward J R, Martinez F J, Huffnagle G B: The microbiome and the respiratory tract. Annual review of physiology 2016, 78:481-504.
20. Cozens D, Sutherland E, Lauder M, Taylor G, Berry C C, Davies R L: Interactions of pathogenic and commensal strains of Mannheimia haemolytica with differentiated bovine airway epithelial cells grown at an air-liquid interface. bioRxiv 2017:197947.
21. Holman D B, Timsit E, Amat S, Abbott D W, Buret A G, Alexander T W: The nasopharyngeal microbiota of beef cattle before and after transport to a feedlot. BMC microbiology 2017, 17:70.
22. Kanmani P, Clua P, Vizoso-Pinto M G, Rodriguez C, Alvarez S, Melnikov V, Takahashi H, Kitazawa H, Villena J: Respiratory commensal bacteria Corynebacterium pseudodiphtheriticum improves resistance of infant mice to respiratory syncytial virus and Streptococcus pneumoniae superinfection. Frontiers in microbiology 2017, 8:1613.
23. Bond S L, Timsit E, Workentine M, Alexander T, Léguillette R: Upper and lower respiratory tract microbiota in horses: bacterial communities associated with health and mild asthma (inflammatory airway disease) and effects of dexamethasone. BMC microbiology 2017, 17:184.
24. Kostric M, Milger K, Krauss-Etschmann S, Engel M, Vestergaard G, Schloter M, Schöler A: Development of a stable lung microbiome in healthy neonatal mice. Microbial ecology 2018, 75:529-542.
25. Bürki S, Frey J, Pilo P: Virulence, persistence and dissemination of Mycoplasma bovis. Veterinary microbiology 2015, 179:15-22.

1. A method for selecting cows to treat for bovine respiratory disease:
collecting a nasal swab sample from a cow;
measuring the level of at least one biomarker associated with a bacterium of a species from the group consisting of: Fusobacterium mortiferum, Prevotella stercorea, Bacteroides vulgatus, Prevotella oris, Clostridium saudiense, Lactobacillus plantarum, Bacteroides uniformis, [Clostridium] clostridioforme, Lactobacillus mucosae, Gemmiger formicilis, Prevotella copri, Terrisporobacter petrolearius, Blautia obeum, [Clostridium] scindens, Lactobacillus caviae, Ruminococcus lactaris, Catenibacterium mitsuokai, Kineothrix alysoides, Streptococcus pasteurianus, Clostridium butyricum, Lactobacillus gasseri, Holdemanella biformis, Faecalibacterium prausnitzii, Ruminococcus faecis, Fusicatenibacter saccharivorans, [Eubacterium] eligens, Butyricicoccus pullicaecorum, Blautia wexlerae, Ruminiclostridium cellobioparum, Massihprevotella massiliensis, and Prevotellamassilia timonensis;
and analyzing the abundance of the biomarker to determine whether to treat the cow.

2. The method of claim 1, wherein the cow is treated if one or more of the following differences in the abundance of a biomarker associated with a bacterial species is detected relative to the abundance in a healthy cow: a decrease in Fusobacterium mortiferum, decrease in Prevotella stercorea, decrease in Bacteroides vulgatus, decrease in Prevotella oris, decrease or increase in Clostridium saudiense (where a decrease indicates that the cow is likely to get BRD and an increase indicates that the cow has BRD), increase in Lactobacillus plantarum, decrease in Bacteroides uniformis, decrease in [Clostridium] clostridioforme, decrease or increase in Lactobacillus mucosae (where a decrease indicates that the cow has BRD and an increase indicates that the cow is likely to get BRD), decrease in Gemmiger formicilis, decrease in Prevotella copri, decrease in Terrisporobacter petrolearius, increase in Blautia obeum, decrease in [Clostridium] scindens, increase in Lactobacillus caviae, increase in Ruminococcus lactaris, decrease or increase in Catenibacterium mitsuokai (where a decrease indicates that the cow has BRD and an increase indicates that the cow is likely to get BRD), increase in Kineothrix alysoides, increase in Streptococcus pasteurianus, increase in Clostridium butyricum, decrease in Lactobacillus gasseri, decrease in Holdemanella biformis, decrease in Faecalibacterium prausnitzii, decrease in Ruminococcus faecis, decrease in Fusicatenibacter saccharivorans, decrease in [Eubacterium] eligens, decrease in Butyricicoccus pullicaecorum, decrease in Blautia wexlerae, increase in Ruminiclostridium cellobioparum, decrease in Massiliprevotella massiliensis, or decrease in Prevotellamassilia timonensis.

3. The method of claim 1, wherein the measured biomarker is associated with a bacterium of one or more of following strains: Fusobacterium mortiferum strain DSM 19809, Prevotella stercorea DSM 18206 strain CB35, Bacteroides vulgatus ATCC 8482, Prevotella oris strain JCM 12252, Clostridium saudiense strain JCC, Lactobacillus plantarum strain CIP 103151, Bacteroides uniformis strain JCM 5828, [Clostridium] clostridioforme strain ATCC 25537, Lactobacillus mucosae strain S32, Gemmiger formicilis strain X2-56, Prevotella copri DSM 18205 strain JCM 13464, Terrisporobacter petrolearius strain LAMOA37, Blautia obeum ATCC 29174, [Clostridium] scindens strain ATCC 35704, Lactobacillus caviae strain MOZM2, Ruminococcus lactaris ATCC 29176, Catenibacterium mitsuokai strain DSM 15897, Kineothrix alysoides strain KNHs209, Streptococcus pasteurianus strain CIP 107122, Clostridium butyricum strain JCM 1391, Lactobacillus gasseri ATCC 33323=JCM 1131, Holdemanella biformis strain DSM 3989, Faecalibacterium prausnitzii strain ATCC 27768, Ruminococcus faecis JCM 15917 strain Eg2, Fusicatenibacter saccharivorans strain HT03-11, [Eubacterium] eligens ATCC 27750, Butyricicoccus pullicaecorum strain 25-3, Blautia wexlerae DSM 19850, Ruminiclostridium cellobioparum DSM 1351=ATCC 15832 strain JCM 1422, Massiliprevotella massiliensis strain Marseille-P2439, and Prevotellamassilia timonensis strain Marseille-P2831.

4. The method of claim 1, wherein the measured biomarker is a biomarker associated with a bacterium from the species Lactobacillus plantarum.

5. The method of claim 4, wherein the cow is treated if the biomarker associated with Lactobacillus plantarum is detected.

6. A method for selecting cows to treat for bovine respiratory disease:
collecting a nasopharyngeal swab sample from a cow;
measuring the level of at least one biomarker associated with a bacterium of a species from the group consisting of: Streptococcus uberis, Salmonella enterica, Kingella negevensis, Prevotella copri, Streptococcus pluranimalium, Holdemanella biformis, Veillonella dispar, Collinsella aerofaciens, Ruminococcus bromii, Prevotella oris, Fournierella massiliensis, Bacteroides plebeius, Lactobacillus mucosae, Alistipes finegoldii, Ruminococcus faecis, Gemmiger formicilis, Butyricicoccus pullicaecorum, Blautia wexlerae, Faecalibacterium prausnitzii, Dorea formicigenerans, Blautia obeum, Bacteroides fragilis, Coprococcus comes, Blautia luti, Dorea longicatena, [Ruminococcus] gnavus, [Eubacterium] hallii, Schaalia cardiffensis, Prevotella stercorea, and Clostridium perfringens;
and analyzing the abundance of the biomarker to determine whether to treat the cow.

7. The method of claim 6, wherein the cow is treated if one or more of the following differences in the abundance of a biomarker associated with a bacterial species is detected relative to the abundance in a healthy cow: an increase in Streptococcus uberis, increase in Salmonella enterica, decrease in Kingella negevensis, decrease or increase in Prevotella copri depending on the 16S rRNA sequence (where a decrease in OTU24 indicates that the cow is likely to get BRD and an increase indicates that the cow has BRD), increase in Streptococcus pluranimalium, decrease in Holdemanella biformis, decrease in Veillonella dispar, decrease in Collinsella aerofaciens, decrease in Ruminococcus bromii, decrease in Prevotella oris, decrease in Fournierella massiliensis, decrease in Bacteroides plebeius, decrease in Lactobacillus mucosae, decrease in Alistipes finegoldii, increase in Ruminococcus faecis, increase in Gemmiger formicilis, increase in Butyricicoccus pullicaecorum, increase in Blautia wexlerae, increase in Faecalibacterium prausnitzii, increase in Dorea formicigenerans, increase in Blautia obeum, decrease in Bacteroides fragilis, increase in Coprococcus comes, increase in Blautia luti, increase in Dorea longicatena, decrease or increase in [Ruminococcus] gnavus depending on the 16S rRNA sequence, increase in [Eubacterium] hallii, decrease in Schaalia cardiffensis, increase in Prevotella stercorea, or decrease in Clostridium perfringens.

8. The method of claim 6, wherein the measured biomarker is associated with a bacterium of one or more of following strains: Streptococcus uberis strain JCM 5709, Salmonella enterica subspecies enterica serovar Typhimurium strain ATCC 13311, Kingella negevensis strain Sch538, Prevotella copri DSM 18205 strain JCM 13464, Streptococcus pluranimalium strain T70, Holdemanella biformis strain DSM 3989, Veillonella dispar strain ATCC 17748, Collinsella aerofaciens strain JCM 10188, Ruminococcus bromii strain ATCC 27255, Prevotella oris strain JCM 12252, Fournierella massiliensis strain AT2, Bacteroides plebeius DSM 17135 strain M12, Lactobacillus mucosae strain S32, Alistipes finegoldii strain DSM 17242, Ruminococcus faecis JCM 15917 strain Eg2, Gemmiger formicilis strain X2-56, Butyricicoccus pullicaecorum strain 25-3, Blautia wexlerae DSM 19850, Faecalibacterium prausnitzii strain ATCC 27768, Dorea formicigenerans strain ATCC 27755, Blautia obeum ATCC 29174, Bacteroides fragilis strain NCTC 9343, Coprococcus comes ATCC 27758, Blautia luti strain BInIX, Dorea longicatena strain 111-35, [Ruminococcus] gnavus ATCC 29149, [Eubacterium] hallii strain ATCC 27751, Schaalia cardiffensis strain CCUG 44997, Prevotella stercorea DSM 18206 strain CB35, and Clostridium perfringens ATCC 13124.

9. The method of claim 6, wherein the measured biomarker is OTU365 (SEQ ID NO: 69) or OTU24 (SEQ ID NO:11) or is a biomarker associated with a bacterium from the genus Kingella, Alistipes, Gemmiger, or Dorea or a bacterium from the species Ruminococcus faecis, Blautia obeum, Blautia luti, or Prevotella stercorea.

10. The method of claim 9, wherein the cow is treated if the biomarker associated with Kingella or Alistipes is not detected, and wherein the cow is treated if OTU365 (SEQ ID NO:69), OTU24 (SEQ ID NO:11), or the biomarker associated with Gemmiger, Dorea, Ruminococcus faecis, Blautia obeum, Blautia luti, Prevotella stercorea is detected.

11. A method for selecting cows to treat for bovine respiratory disease:
collecting a bronchoalveolar lavage sample from a cow;
measuring the level of at least one biomarker associated with a bacterium of a species from the group consisting of: Mycoplasma dispar, Mannheimia haemolytica, Moraxella caviae, Micrococcus luteus, Massilia agri, Terrimonas lutea, Alkalibacter saccharofermentans, [Clostridium] glycyrrhizinilyticum, Flavobacterium acidificum, Alistipes putredinis, Collinsella aerofaciens, Solibacillus isronensis, Monoglobus pectinilyticus, Caldalkalibacillus thermarum, Solitalea canadensis, Anaerostipes caccae, Eisenbergiella massiliensis, Olsenella profuse, Dorea formicigenerans, Blautia wexlerae, [Eubacterium] rectale, Pseudomonas lini, Prevotella shahii, Kroppenstedtia pulmonis, Geosporobacter ferrireducens, Mediterranea massiliensis, Staphylococcus aureus, Schaalia cardiffensis, Flavonifractor plautii, Butyricimonas virosa, Streptococcus pasteurianus, Haemophilus sputorum, and Lactobacillus mucosae;
and analyzing the abundance of the biomarker to determine whether to treat the cow.

12. The method of claim 11, wherein the cow is treated if one or more of the following differences in the abundance of a biomarker associated with a bacterial species is detected relative to the abundance in a healthy cow: a decrease in Mycoplasma dispar, decrease in Mannheimia haemolytica, decrease in Moraxella caviae, decrease in Micrococcus luteus, decrease in Massilia agri, decrease in Terrimonas lutea, increase in Alkalibacter saccharofermentans, increase in [Clostridium] glycyrrhizinilyticum, decrease in Flavobacterium acidificum, decrease in Alistipes putredinis, increase in Collinsella aerofaciens, decrease in Solibacillus isronensis, decrease in Monoglobus pectinilyticus, increase in Caldalkalibacillus thermarum, increase in Solitalea canadensis, increase in Anaerostipes caccae, decrease in Eisenbergiella massiliensis, decrease in Olsenella profuse, increase in Dorea formicigenerans, increase in Blautia wexlerae, decrease in [Eubacterium] rectale, decrease in Pseudomonas lini, decrease in Prevotella shahii, increase in Kroppenstedtia pulmonis, decrease in Geosporobacter ferrireducens, increase in Mediterranea massiliensis, increase in Staphylococcus aureus, decrease in Schaalia cardiffensis, increase in Flavonifractor plautii, decrease in Butyricimonas virosa, decrease in Streptococcus pasteurianus, decrease in Haemophilus sputorum, and decrease in Lactobacillus mucosae.

13. The method of claim 11, wherein the measured biomarker is associated with a bacterium of one or more of following strains: Mycoplasma dispar strain 462/2, Mannheimia haemolytica strain NCTC 9380, Moraxella caviae strain GP11, Micrococcus luteus strain NCTC 2665, Massilia agri strain K-3-1, Terrimonas lutea strain DY, Alkalibacter saccharofermentans strain Z-79820, [Clostridium] glycyrrhizinilyticum strain ZM35, Flavobacterium acidificum strain LMG 8364, Alistipes putredinis strain JCM 16772, Collinsella aerofaciens strain JCM 10188, Solibacillus isronensis B3W22, Monoglobus pectinilyticus strain 14, Caldalkalibacillus thermarum strain HA6, Solitalea canadensis DSM 3403, Anaerostipes caccae strain L1-92, Eisenbergiella massiliensis strain AT11, Olsenella profusa DSM 13989, Dorea formicigenerans strain ATCC 27755, Blautia wexlerae DSM 19850, [Eubacterium] rectale ATCC 33656, Pseudomonas lini strain DLE411J, Prevotella shahii strain EHS11, Kroppenstedtia pulmonis strain W9323, Geosporobacter ferrireducens strain IRF9, Mediterranea massiliensis strain Marseille-P2645, Staphylococcus aureus strain S33 R, Schaalia cardiffensis strain CCUG 44997, Flavonifractor plautii strain Prevot S1, Butyricimonas virosa strain MT12, Streptococcus pasteurianus strain CIP 107122, Haemophilus sputorum CCUG 13788, and Lactobacillus mucosae strain S32.

14. The method of claim 11, wherein the measured biomarker is a biomarker associated with a bacterium from the genus Moraxella.

15. The method of claim 14, wherein the cow is treated if the biomarker associated with Moraxella is not detected.

16. The method of claim 1, wherein the step of measuring the level of a biomarker comprises detecting a protein associated with a particular bacterium using an antibody-based method.

17. The method of claim 1, wherein the step of measuring the level of a biomarker comprises detecting a nucleic acid associated with a particular bacterium.

18. (canceled)

19. The method of claim 17, wherein the nucleic acid is a component of a 16S or 23S ribosomal subunit.

20. The method of claim 19, wherein the nucleic acid comprises a sequence listed in Tables 2-7.

21. (canceled)

22. The method of claim 1, wherein the sample is collected from a cow following transportation to a new location or feedlot.

23.-35. (canceled)


SEQUENCE LISTING (86 sequences)

SEQ ID NO: 1 - DNA, 252 bp, Fusobacterium mortiferum
tacgtatgtcgcaagcgttatccggatttattgggcgtaaagcgcgtctaggcggtttggtaagtctgatgtgaaaatgcggggctcaactccgtattgcgttggaaactgccaaactagagtactggagaggtgggcggaactacaagtgtagaggtgaaattcgtagatatttgtaggaatgccgatggggaagccagcccactggacagatactgacgctaaagcgcgaaagcgtgggtagcaaacagg

SEQ ID NO: 2 - DNA, 253 bp, Prevotella stercorea
tacggaaggtccgggcgttatccggatttattgggtttaaagggagcgtaggccgtctgttaagcgtgttgtgaaatgtcggggctcaacctgggcattgcagcgcgaactggcagacttgagtgcgcgggaagtaggcggaattcgtcgtgtagcggtgaaatgcttagatatgacgaagaactccgattgcgaaggcagcctgctgtagtgcaactgacgctgaagctcgaaagcgtgggtatcgaacagg

SEQ ID NO: 3 - DNA, 253 bp, Bacteroides vulgatus
tacggaggatccgagcgttatccggatttattgggtttaaagggagcgtagatggatgtttaagtcagttgtgaaagtttgcggctcaaccgtaaaattgcagttgatactggatatcttgagtgcagttgaggcaggcggaattcgtggtgtagcggtgaaatgcttagatatcacgaagaactccgattgcgaaggcagcctgctaagctgcaactgacattgaggctcgaaagtgtgggtatcaaacagg

SEQ ID NO: 4 - DNA, 253 bp, Prevotella oris
tacggaaggtccgggcgttatccggattcattgggtttaaagggagcgcaggccgccccttaagcgtgttgtgaaatgccgcggctcaaccgtggcactgcagcgcgaactggggggcttgagtgcgcgcagcgcaggcggaactcgtggtgtagcggtgaaatgcttagatatcacgaagaactccgatcgcgaaggcagcctgcgggagcgcaactgacgctgaggctcgaaagtgcgggtatcgaacagg

SEQ ID NO: 5 - DNA, 253 bp, Clostridium saudiense
tacgtaggtggcgagcgttgtccggatttactgggcgtaaagggagcgtaggcggatttttaagtgagatgtgaaatactcgggcttaacctgagtgctgcatttcaaactggaagtctagagtgcaggagaggagaagggaattcctagtgtagcggtgaaatgcgtagagattaggaagaacaccagtggcgaaggcgcttctctggactgtaactgacgctgaggctcgaaagcgtggggagcaaacagg

SEQ ID NO: 6 - DNA, 253 bp, Lactobacillus plantarum
tacgtaggtggcaagcgttgtccggatttattgggcgtaaagcgagcgcaggcggttttttaagtctgatgtgaaagccttcggctcaaccgaagaagtgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagtatgggtagcaaacagg

SEQ ID NO: 7 - DNA, 253 bp, Bacteroides uniformis
tacggaggatccgagcgttatccggatttattgggtttaaagggagcgtaggcggacgcttaagtcagttgtgaaagtttgcggctcaaccgtaaaattgcagttgatactgggtgtcttgagtacagtagaggcaggcggaattcgtggtgtagcggtgaaatgcttagatatcacgaagaactccgattgcgaaggcagcttgctggactgtaactgacgctgatgctcgaaagtgtgggtatcaaacagg

SEQ ID NO: 8 - DNA, 253 bp, [Clostridium] clostridioforme
tacgtagggggcaagcgttatccggatttactgggtgtaaagggagcgtagacggcgaagcaagtctgaagtgaaaacccagggctcaaccctgggactgctttggaaactgttttgctagagtgtcggagaggtaagtggaattcctagtgtagcggtgaaatgcgtagatattaggaggaacaccagtggcgaaggcggcttactggacgataactgacgttgaggctcgaaagcgtggggagcaaacagg

SEQ ID NO: 9 - DNA, 253 bp, Lactobacillus mucosae
tacgtaggtggcaagcgttatccggatttattgggcgtaaagcgagcgcaggcggtttgataagtctgatgtgaaagcctttggcttaaccaaagaagtgcatcggaaactgtcagacttgagtgcagaagaggacagtggaactccatgtgtagcggtggaatgcgtagatatatggaagaacaccagtggcgaaggcggctgtctggtctgcaactgacgctgaggctcgaaagcatgggtagcgaacagg

SEQ ID NO: 10 - DNA, 253 bp, Gemmiger formicilis
aacgtagggtgcaagcgttgtccggaattactgggtgtaaagggagcgcaggcggaccggcaagttggaagtgaaaaccatgggctcaacccataaattgctttcaaaactgctggccttgagtagtgcagaggtaggtggaattcccggtgtagcggtggaatgcgtagatatcgggaggaacaccagtggcgaaggcgacctactgggcaccaactgacgctgaggctcgaaagcatgggtagcaaacagg

SEQ ID NO: 11 - DNA, 253 bp, Prevotella copri
tacggaaggtccgggcgttatccggatttattgggtttaaagggagcgtaggccggagattaagcgtgttgtgaaatgtagtggctcaacctctgcactgcagcgcgaactggtcttcttgagtacgcacaacgtgggcggaattcgtggtgtagcggtgaaatgcttagatatcacgaagaactccgattgcgaaggcagctcacgggagcgcaactgacgctgaagctcgaaagtgcgggtatcgaacagg

SEQ ID NO: 12 - DNA, 252 bp, Terrisporobacter petrolearius
tacgtagggggctagcgttatccggatttactgggcgtaaagggtgcgtaggtggtttcttaagtcaggagtgaaaggctacggctcaaccgtagtaagctcttgaaactgggaaacttgagtgcaggagaggaaagtggaattcctagtgtagcggtgaaatgcgtagatattaggaggaacaccagtagcgaaggcggctttctggactgtaactgacactgaggcacgaaagcgtggggagcaaacagg

SEQ ID NO: 13 - DNA, 253 bp, Prevotella oris
tacggaaggtccgggcgttatccggatttattgggtttaaagggagcgtaggccgtcttttaagcgtgttgtgaaatactgtcgctcaacgacagaggtgcagcgcgaactgggagacttgagtgcgcggaatgcaggcggaattcgtcgtgtagcggtgaaatgcttagatatgacgaagaactccgattgcgaaggcagcttgcagtagcgtaactgacgctgaagctcgaaagtgcgggtatcgaacagg

SEQ ID NO: 14 - DNA, 253 bp, Blautia obeum
tacgtagggggcaagcgttatccggatttactgggtgtaaagggagcgtagacggactggcaagtctgatgtgaaaggcgggggctcaacccctggactgcattggaaactgttagtcttgagtgccggagaggtaagcggaattcctagtgtagcggtgaaatgcgtagatattaggaggaacaccagtggcgaaggcggcttactggacggtaactgacgttgaggctcgaaagcgtggggagcaaacagg

SEQ ID NO: 15 - DNA, 253 bp, [Clostridium] scindens
tacgtagggggcaagcgttatccggatttactgggtgtaaagggagcgtagacggcgatgcaagccagatgtgaaagcccggggctcaaccccgggactgcatttggaactgcgtggctggagtgtcggagaggcaggcggaattcctagtgtagcggtgaaatgcgtagatattaggaggaacaccagtggcgaaggcggcctgctggacgatgactgacgttgaggctcgaaagcgtggggagcaaacagg

SEQ ID NO: 16 - DNA, 253 bp, Lactobacillus caviae
tacgtaggtggcaagcgttatccggatttattgggcgtaaagcgagcgcaggcggttgcttaggtctgatgtgaaagccttcggcttaaccgaagaagtgcatcggaaaccgggcgacttgagtgcagaagaggacagtggaactccatgtgtagcggtggaatgcgtagatatatggaagaacaccagtggcgaaggcggctgtctggtctgcaactgacgctgaggctcgaaagcatgggtagcgaacagg

SEQ ID NO: 17 - DNA, 253 bp, Ruminococcus lactaris
tacgtagggggcaagcgttatccggatttactgggtgtaaagggagcgcagacggaactgcaagtctgatgtgaaagcccggggctcaaccgcgggactgcatcggatactgtagatctggagtgccggaggggcaagcggaattcctggtgtagcggtgaaatgcgtagatatcaggaggaacaccggcggcgaaggcggcttgctggacggcaactgacgttgaggctcgaaagcgtggggagcaaacagg

SEQ ID NO: 18 - DNA, 252 bp, Catenibacterium mitsuokai
tacgtaggtggcgagcgttatccggaatcattgggcgtaaagagggagcaggcggccgcaagggtctgtggtgaaagaccgaagctaaacttcggtgagccatggaaaccgggcggctagagtgcggaagaggatcgtggaattccatgtgtagcggtgaaatgcgtagatatatggaggaacaccagtggcgaaggcgacggtctgggccgcaactgacgctcattcccgaaagcgtggggagcaaatagg

SEQ ID NO: 19 - DNA, 253 bp, Kineothrix alysoides
tacgtagggggcaagcgttatccggatttactgggtgtaaagggagcgtagacggcaaggcaagtctgatgtgaaataccggggctcaaccccgggactgcattggaaactgtttagctagagtgcaggagaggtaagtggaattcctagtgtagcggtgaaatgcgtagatattaggaggaacaccagtggcgaaggcggcttactggactgtaactgacgttgaggctcgaaagcgtggggagcaaacagg

SEQ ID NO: 20 - DNA, 252 bp, Streptococcus pasteurianus
tacgtaggtcccgagcgttgtccggatttattgggcgtaaagcgagcgcaggcggtttaataagtctgaagttaaaggcagtggcttaaccattgttcgctttggaaactgttaaacttgagtgcagaaggggagagtggaattccatgtgtagcggtgaaatgcgtagatatatggaggaacaccggtggcgaaagcggctctctggtctgtaactgacgctgaggctcgaaagcgtggggagcaaacagg

SEQ ID NO: 21 - DNA, 252 bp, Streptococcus uberis
tacgtaggtcccgagcgttgtccggatttattgggcgtaaagcgagcgcaggcggtttgataagtctgaagttaaaggctgtggcttaaccatagttcgctttggaaactgtcaaacttgagtgcagaaggggagagtggaattccatgtgtagcggtgaaatgcgtagatatatggaggaacaccggtggcgaaagcggctctctggtctgtaactgacgctgaggctcgaaagcgtggggagcaaacagg

SEQ ID NO: 22 - DNA, 253 bp, Salmonella enterica
tacggagggtgcaagcgttaatcggaattactgggcgtaaagcgcacgcaggcggtctgtcaagtcggatgtgaaatccccgggctcaacctgggaactgcattcgaaactggcaggcttgagtcttgtagaggggggtagaattccaggtgtagcggtgaaatgcgtagagatctggaggaataccggtggcgaaggcggccccctggacaaagactgacgctcaggtgcgaaagcgtggggagcaaacagg

SEQ ID NO: 23 - DNA, 253 bp, Kingella negevensis
tacgtagggtgcaagcgttaatcggaattactgggcgtaaagcgagcgcagacggttatttaagtcagatgtgaaatccccgagctcaacttgggaactgcgtttgaaactggatagctagagtgtgtcagaggggggtagaattccacgtgtagcagtgaaatgcgtagagatgtggaggaataccgatggcgaaggcagccccctgggataacactgacgttcatgctcgaaagcgtgggtagcaaacagg

SEQ ID NO: 24 - DNA, 252 bp, Streptococcus pluranimalium
tacgtaggtcccgagcgttgtccggatttattgggcgtaaagcgagcgcaggtggtttaataagtctgaagttaaaggcattggctcaaccaatgtacgctttggaaactgttaaacttgagtgcagaaggggagagtggaattccatgtgtagcggtgaaatgcgtagatatatggaggaacaccggtggcgaaagcggctctctggtctgtaactgacactgaggctcgaaagcgtgggtagcgaacagg

SEQ ID NO: 25 - DNA, 253 bp, Holdemanella biformis
tacgtaggtggcgagcgttatccggaatgattgggcgtaaagggtgcgtaggtggcagatcaagtctggagtaaaaggtatgggctcaacccgtactggctctggaaactgatcagctagagaacagaagaggacggcggaactccatgtgtagcggtaaaatgcgtagatatatggaagaacaccggtggcgaaggcggccgtctggtctggattctgacactgaagcacgaaagcgtggggagcaaatagg

SEQ ID NO: 26 - DNA, 252 bp, Veillonella dispar
tacgtaggtggcaagcgttgtccggaattattgggcgtaaagcgcgcgcaggcggatttgtcagtctgtcttaaaagttcggggcttaaccccgtgaggggatggaaactacaaatctagagtatcggagaggaaagtggaattcctagtgtagcggtgaaatgcgtagatattaggaagaacaccggtggcgaaggcgactttctggacgaaaactgacgctgaggcgcgaaagccaggggagcgaacggg

SEQ ID NO: 27 - DNA, 253 bp, Collinsella aerofaciens
tacgtagggggcgagcgttatccggattcattgggcgtaaagcgcgcgtaggcggcccggcaggccgggggtcgaagcggggggctcaaccccccgaagcccccggaacctccgcggcttgggtccggtaggggagggtggaacacccggtgtagcggtggaatgcgcagatatcgggtggaacaccggtggcgaaggcggccctctgggccgagaccgacgctgaggcgcgaaagctgggggagcgaacagg

SEQ ID NO: 28 - DNA, 253 bp, Ruminococcus bromii
tacgtagggagcaagcgttgtccggatttactgggtgtaaagggtgcgtaggcggctttgcaagtcagatgtgaaatctatgggctcaacccataaactgcatttgaaactgtagagcttgagtgaagtagaggcaggcggaattccccgtgtagcggtgaaatgcgtagagatggggaggaacaccagtggcgaaggcggcctgctgggctttaactgacgctgaggcacgaaagcgtgggtagcaaacagg

SEQ ID NO: 29 - DNA, 253 bp, Prevotella oris
tacggaaggtccgggcgttatccggatttattgggtttaaagggagcgtaggccggagattaagcgtgttgtgaaatgccgcggctcaaccgtggcactgcagcgcgaactggttttcttgagtgcgcgcgacgcaggcggaattcgtggtgtagcggtgaaatgcttagatatcacgaggaaccccgattgcgaaggcagcttgcgggagcgcaactgacgctgaagctcgaaggtgcgggtatcgaacagg

SEQ ID NO: 30 - DNA, 253 bp, Fournierella massiliensis
aacgtaggtggcaagcgttgtccggaattactgggtgtaaagggagcgcaggcggaaggacaagttggaagtgaaacccacgggctcaacccgtgaactgctttcaaaactgtttttcttgagtggtgtagaggtaggcggaattcccggtgtagcggtggaatgcgtagatatcgggaggaacaccagtggcgaaggcggcctactgggcactaactgacgctgaggctcgaaagcatgggtagcaaacagg

SEQ ID NO: 31 - DNA, 253 bp, Bacteroides plebeius
tacggaggatgcgagcgttatccggatttattgggtttaaagggagcgtagacgggatgttaagtcagttgtgaaaggctgcggctcaaccgcagcactgcagttgatactggcgtccttgagtgcggttgaggtgtgtggaattcgtggtgtagcggtgaaatgcttagatatcacgaagaactccgattgcgaaggcagcacactaagccgctactgacgttgaggctcgaaagtgtgggtatcaaacagg

SEQ ID NO: 32 - DNA, 253 bp, Prevotella copri
tacggaaggtccgggcgttatccggatttattgggtttaaagggagcgtaggccggagggtaagcgtgttgtgaaatgcagttgctcaacatctgcactgcagcgcgaactgttctccttgagtgcgcaggaagtaggcggaattcgtcgtgtagcggtgaaatgcttagatatgacgaagaactccgattgggaagccagcttactgtagcgccactgacgctgatgctcgaaagtgcgggtatcgaacagg

SEQ ID NO: 33 - DNA, 253 bp, Alistipes finegoldii
tacggaggatccaagcgttatccggatttattgggtttaaagggtgcgtaggcggattagtaagttagaggtgaaagctcggggctcaactccggaactgcctctgatactgctgatctagagagtagatgcggtaggcggaatgtatggtgtagcggtgaaatgcttagagatcatacagaacaccgattgcgaaggcagcttaccaaactatatctgacgttgaggcacgaaagcgtggggagcaaacagg

SEQ ID NO: 34 - DNA, 253 bp, Mycoplasma dispar
tacataggtcgcaagcgttatccggaattattgggcgtaaagcgtccgtaggttttttgttaagtttaaggttaaatgctaaagctcaactttagtccgctttagatactggcaaaatagaattatgaagaggttagcggaattcctagtggagcggtggaatgcgtagatattaggaagaacaccaataggcgaaggcagctaactggtcatatattgacactaagggacgaaagcgtggggagcaaacagg

SEQ ID NO: 35 - DNA, 253 bp, Mannheimia haemolytica
tacagagggtgcaagcgttaatcggaataactgggcgtaaagggcacgcaggcggttgtttaagtgaggtgtgaaagccccgggcttaacctgggaattgcatttcagactgaacaactagagtactttagggaggggtagaattccacgtgtagcggtgaaatgcgtagagatgtggaggaataccgaaggcgaaggcagccccttggaaatgtactgacgctcatgtgcgaaagcgtggggagcaaacagg

SEQ ID NO: 36 - DNA, 253 bp, Moraxella caviae
tacagagggtgcaagcgttaatcggaattactgggcgtaaagcgagcgtaggtggttgtttaagtcagatgtgaaagccccgggcttaacctgggaactgcatctgatactggacaactagagtaggtgagagggaagtagaattccaggtgtagcggtgaaatgcgtagagatctggaggaataccgatggcgaaggcagcttcctggcatcatactgacactgaggttcgaaagcgtgggtagcaaacagg

SEQ ID NO: 37 - DNA, 253 bp, Micrococcus luteus
tacgtagggtgcgagcgttatccggaattattgggcgtaaagagctcgtaggcggtttgtcgcgtctgtcgtgaaagtccggggcttaaccccggatctgcggtgggtacgggcagactagagtgcagtaggggagactggaattcctggtgtagcggtggaatgcgcagatatcaggaggaacaccgatggcgaaggcaggtctctgggctgtaactgacgctgaggagcgaaagcatggggagcgaacagg

SEQ ID NO: 38 - DNA, 253 bp, Massilia agri
tacgtagggtgcaagcgttaatcggaattactgggcgtaaagcgtgcgcaggcggttttgtaagtctgacgtgaaagccccgggctcaacctgggaattgcgttggagactgcaaggcttgaatctggcagaggggggtagaattccacgtgtagcagtgaaatgcgtagagatgtggaggaacaccgatggcgaaggcagccccctgggtcaagattgacgctcatgcacgaaagcgtggggagcaaacagg

SEQ ID NO: 39 - DNA, 253 bp, Terrimonas lutea
tacggagggtgcaagcattatccggatttattgggtttaaagggtgcgtaggtggctaggtaagtcagtggtgaaatccccaagcttaacttgggaactgccattgatactatctagcttgaatgttgaggaggtgagcagaatatatcatgtagcggtgaaatgctcagatatgatatagaataccgattgcgaaggccgctcactacacaactattgacactgaggcacgaaagcgtggggatcaaacagg

SEQ ID NO: 40 - DNA, 253 bp, Alkalibacter saccharofermentans
tacgtagggggcaagcgttgtccggaatgactgggcgtaaagggcgtgtaggcggctttttaagtgtgaagtgaaagtcctgctttcaaggtgggaactgctttgcaaactggagagcttgagtgcggaagaggtaagtggaattcccagtgtagcggtgaaatgcgtagagattgggaggaacaccagtggcgaaggcgacttactgggccgtaactgacgctgaggcgcgaaagcgtggggagcgaacagg

SEQ ID NO: 41 - DNA, 253 bp, [Clostridium] glycyrrhizinilyticum
tacgtagggggcaagcgttatccggatttactgggtgtaaagggagcgtagacggaagagcaagtctgatgtgaaaacccggggctcaaccccgggactgcattggaaactgtttttctggagtgtcggagaggtaagcggaattcctagtgtagcggtgaaatgcgtagatattaggaggaacaccagtggcgaaggcggcttactggacgatcactgacgttgaggctcgaaagcgtggggagcaaacagg

SEQ ID NO: 42 - DNA, 253 bp, Flavobacterium acidificum
tacggagggtgcaagcgttaatcggaattactgggcgtaaagcgcacgcaggcggtctgttaagtcagatgtgaaatccccgggcttaacctgggaactgcatttgaaactggcaggcttgagtctcgtagaggggggtagaattccaggtgtagcggtgaaatgcgtagagatctggaggaataccggtggcgaaggcggccccctggacgaagactgacgctcaggtgcgaaagcgtggggagcaaacagg

SEQ ID NO: 43 - DNA, 253 bp, Alistipes putredinis
tacggaggatccaagcgttatccggatttattgggtttaaagggtgcgtaggcggtttggtaagttagaggtgaaatttcagggctcaaccttgacattgcctctgatactgtcgaactagagagtagttgctgtgggcggaatgtatggtgtagcggtgaaatgcttagagatcatacagaacaccgattgcgaaggcagctcacaaaactatatctgacgttgaggcacgaaagcgtgggtagcaaacagg

SEQ ID NO: 44 - DNA, 253 bp, Solibacillus isronensis; identical to SEQ ID NO: 27

SEQ ID NO: 45 - DNA, 253 bp, Monoglobus pectinilyticus
tacgtaggtggcaagcgttgtccggatttactgggtgtaaagggcgtgtaggcgggctgataagtcagatgtgaaataccggggcttaaccccggggctgcatttgaaactgtcagtcttgagtgtcggagaggtaagcggaattcctagtgtagcggtgaaatgcgtagatattaggaggaacaccagtggcgaaggcggcttactggacgataactgacgctgaggcgcgaaagcgtgggtagcaaacagg

SEQ ID NO: 46 - DNA, 252 bp, Clostridium butyricum; identical to SEQ ID NO: 1

SEQ ID NO: 47 - DNA, 253 bp, Lactobacillus gasseri
tacgtaggtggcaagcgttgtccggatttattgggcgtaaagcgagtgcaggcggttcaataagtctgatgtgaaagccttcggctcaaccggagaattgcatcagaaactgttgaacttgagtgcagaagaggagagtggaactccatgtgtagcggtggaatgcgtagatatatggaagaacaccagtggcgaaggcggctctctggtctgcaactgacgctgaggctcgaaagcatgggtagcgaacagg

SEQ ID NO: 48 - DNA, 253 bp, Clostridium saudiense
tacgtaggtggcgagcgttgtccggatttactgggcgtaaagggagcgtaggcggacttttaagtgagatgtgaaatacccgggctcaacttgggtgctgcatttcaaactggaagtctagagtgcaggagaggagaatggaattcctagtgtagcggtgaaatgcgtagagattaggaagaacaccagtggcgaaggcgattctctggactgtaactgacgctgaggctcgaaagcgtggggagcaaacagg

SEQ ID NO: 49 - DNA, 253 bp, Faecalibacterium prausnitzii
aacgtaggtcacaagcgttgtccggaattactgggtgtaaagggagcgcaggcgggaagacaagttggaagtgaaatccatgggctcaacccatgaactgctttcaaaactgtttttcttgagtagtgcagaggtaggcggaattcccggtgtagcggtggaatgcgtagatatcgggaggaacaccagtggcgaaggcggcctactgggcaccaactgacgctgaggctcgaaagtgtgggtagcaaacagg

SEQ ID NO: 50 - DNA, 253 bp, Ruminococcus faecis
tacgtagggggcaagcgttatccggatttactgggtgtaaagggagcgtagacggaatggcaagtctgatgtgaaaggccggggctcaaccccgggactgcattggaaactgtcaatctagagtaccggaggggtaagtggaattcctagtgtagcggtgaaatgcgtagatattaggaggaacaccagtggcgaaggcggcttactggacggtaactgacgttgaggctcgaaagcgtggggagcaaacagg

SEQ ID NO: 51 - DNA, 253 bp, Fusicatenibacter saccharivorans
tacgtagggggcaagcgttatccggatttactgggtgtaaagggagcgtagacggcaaggcaagtctgatgtgaaaacccagggcttaaccctgggactgcattggaaactgtctggctcgagtgccggagaggtaagcggaattcctagtgtagcggtgaaatgcgtagatattaggaagaacaccagtggcgaaggcggcttactggacggtaactgacgttgaggctcgaaagcgtggggagcaaacagg

SEQ ID NO: 52 - DNA, 253 bp, Ruminococcus faecis
tacgtatggtgcaagcgttatccggatttactgggtgtaaagggagcgtagacggagtggcaagtctgatgtgaaaacccggggctcaaccccgggactgcattggaaactgtcaatctagagtaccggagaggtaagcggaattcctagtgtagcggtgaaatgcgtagatattaggaggaacaccagtggcgaaggcggcttactggacggtaactgacgttgaggctcgaaagcgtggggagcaaacagg

SEQ ID NO: 53 - DNA, 253 bp, [Eubacterium] eligens
tacgtatggtgcaagcgttatccggatttactgggtgtaaagggagcgtaggtggcaaggcaagccagaagtgaaaacccggggctcaaccgcgggattgcttttggaactgtcatgctggagtgcaggaggggtgagcggaattcctagtgtagcggtgaaatgcgtagatattaggaggaacaccggaggcgaaggcggctcactggactgtaactgacactgaggctcgaaagcgtggggagcaaacagg

SEQ ID NO: 54 - DNA, 253 bp, Butyricicoccus pullicaecorum
tacgtagggagcaagcgttatccggatttactgggtgtaaagggcgagtaggcggattggcaagttgggagtgaaatgtcggggcttaaccccggaactgcttccaaaactgttgatcttgagtgatggagaggcaggcggaattcccagtgtagcggtgaaatgcgtagatattgggaggaacaccagtggcgaaggcggcctgctggacattaactgacgctgaggagcgaaagcgtggggagcaaacagg

SEQ ID NO: 55 - DNA, 253 bp, Blautia wexlerae
tacgtagggggcaagcgttatccggatttactgggtgtaaagggagcgtagacggtgtggcaagtctgatgtgaaaggcatgggctcaacctgtggactgcattggaaactgtcatacttgagtgccggaggggtaagcggaattcctagtgtagcggtgaaatgcgtagatattaggaggaacaccagtggcgaaggcggcttactggacggtaactgacgttgaggctcgaaagcgtggggagcaaacagg

SEQ ID NO: 56 - DNA, 253 bp, Ruminiclostridium cellobioparum
tacgtaggtggcgagcgttatccggatttactgggtgtaaagggcgcgtaggcgggaatgcaagtcagatgtgaaatccaagggctcaacccttgaactgcatttgaaactgtatttcttgagtgtcggagaggttgacggaattcctagtgtagcggtgaaatgcgtagatattaggaggaacaccagtggcgaaggcggtcaactggacgataactgacgctgaggcgcgaaagcgtggggagcaaacagg

SEQ ID NO: 57 - DNA, 253 bp, Massiliprevotella massiliensis
tacggaaggtccgggcgttatccggatttattgggtttaaagggagcgcaggccgccgggcaagcgtgttgtgaaatgcagtcgctcaacgtctgcactgcagcgcgaactgcccagcttgagtgcgcgcaacgttggcggaattcgccgtgtagcggtgaaatgcttagatatggcgaagaactccgattgcgaaggcagctgacgggtgcgtaactgacgctcatgctcgaaagtgcgggtatcgaacagg

SEQ ID NO: 58 - DNA, 253 bp, Prevotellamassilia timonensis
tacggaaggtccgggcgttatccggatttattgggtttaaagggagcgtaggcgggctattaagtcagctgtgaaatccggcggctcaaccgtcggcctgcagttgatactggtggccttgagtgcgcgcaggggcgttggaattcatggtgtagcggtgaaatgctcagatatcatgaagaactccgatcgcgaaggcagatgcccggagcgcaactgacgctgaggctcgaaagtgcgggtatcaaacagg

SEQ ID NO: 59 - DNA, 253 bp, Dorea formicigenerans
tacgtagggggcaagcgttatccggatttactgggtgtaaagggagcgtagacggctgtgcaagtctgaagtgaaaggcatgggctcaacctgtggactgctttggaaactgtgcagctagagtgtcggagaggtaagtggaattcctagtgtagcggtgaaatgcgtagatattaggaggaacaccagtggcgaaggcggcttactggacgatgactgacgttgaggctcgaaagcgtggggagcaaacagg

SEQ ID NO: 60 - DNA, 253 bp, Bacteroides fragilis
tacggaggatccgagcgttatccggatttattgggtttaaagggagcgtaggtggactggtaagtcagttgtgaaagtttgcggctcaaccgtaaaattgcagttgatactgtcagtcttgagtacagtagaggtgggcggaattcgtggtgtagcggtgaaatgcttagatatcacgaagaactccgattgcgaaggcagctcactggactgcaactgacactgatgctcgaaagtgtgggtatcaaacagg

SEQ ID NO: 61 - DNA, 253 bp, Coprococcus comes
tacgtatggtgcaagcgttatccggatttactgggtgtaaagggagcgtagacggctgtgtaagtctgaagtgaaagcccggggctcaaccccgggactgctttggaaactatgcagctagagtgtcggagaggtaagtggaattcccagtgtagcggtgaaatgcgtagatattgggaggaacaccagtggcgaaggcggcttactggacgatgactgacgttgaggctcgaaagcgtggggagcaaacagg

SEQ ID NO: 62 - DNA, 253 bp, Prevotella copri
tacggaaggtccgggcgttatccggatttattgggtttaaagggagcgtaggccggagattaagcgtgttgtgaaatgtagatgctcaacgtctgcactgcagcgcgaactggtttccttgagtacgcacaaagtgggcggaattcgtggtgtagcggtgaaatgcttagatatcacgaagaactccgattgcgaaggcagctcactggagcgcaactgacgctgaagctcgaaagtgcgggtatcgaacagg

SEQ ID NO: 63 - DNA, 253 bp, Blautia luti
tacgtagggggcaagcgttatccggatttactgggtgtaaagggagcgtagacggatggacaagtctgatgtgaaaggctggggctcaaccccgggactgcattggaaactgcccgtcttgagtgccggagaggtaagcggaattcctagtgtagcggtgaaatgcgtagatattaggaggaacaccagtggcgaaggcggcttactggacggtaactgacgttgaggctcgaaagcgtggggagcaaacagg

SEQ ID NO: 64 - DNA, 253 bp, Dorea longicatena
tacgtagggggcaagcgttatccggatttactgggtgtaaagggagcgtagacggcacggcaagccagatgtgaaagcccggggctcaaccccgggactgcatttggaactgctgagctagagtgtcggagaggcaagtggaattcctagtgtagcggtgaaatgcgtagatattaggaggaacaccagtggcgaaggcggcttgctggacgatgactgacgttgaggctcgaaagcgtggggagcaaacagg

SEQ ID NO: 65 - DNA, 253 bp, [Ruminococcus] gnavus
tacgtagggggcaagcgttatccggatttactgggtgtaaagggagcgtagacggcatggcaagccagatgtgaaagcccggggctcaaccccgggactgcatttggaactgtcaggctagagtgtcggagaggaaagcggaattcctagtgtagcggtgaaatgcgtagatattaggaggaacaccagtggcgaaggcggctttctggacgatgactgacgttgaggctcgaaagcgtggggagcaaacagg

SEQ ID NO: 66 - DNA, 253 bp, [Eubacterium] hallii
tacgtagggagcaagcgttatccggatttactgggtgtaaagggtgcgtaggtggcagtgcaagtcagatgtgaaaggccggggctcaaccccggagctgcatttgaaactgcatagctagagtacaggagaggcaggcggaattcctagtgtagcggtgaaatgcgtagatattaggaggaacaccagtggcgaaggcggcctgctggactgttactgacactgaggcacgaaagcgtggggagcaaacagg

SEQ ID NO: 67 - DNA, 253 bp, Schaalia cardiffensis
tacgtagggcgcgagcgttgtccggaattattgggcgtaaagggcttgtaggcggttggttgcgtctgccgtgaaatcctctggcttaactgggggcgtgcggtgggtacgggctggcttgagtgcggtaggggagactggaattcctggtgtagcggtggaatgcgcagatatcaggaggaacaccggtggcgaaggcgggtctctgggccgttactgacgctgaggagcgaaagcgtggggagcgaacagg

SEQ ID NO: 68 - DNA, 253 bp, Clostridium perfringens
tacgtaggtggcgagcgttatccggatttactgggcgtaaagggagcgtaggcggatgattaagtgggatgtgaaatacccgggctcaacttgggtgctgcattccaaactggttatctagagtgcaggagaggagagtggaattcctagtgtagcggtgaaatgcgtagagattaggaagaacaccagtggcgaaggcgactctctggactgtaactgacgctgaggctcgaaagcgtggggagcaaacagg

SEQ ID NO: 69 - DNA, 253 bp, [Ruminococcus] gnavus; identical to SEQ ID NO: 65

SEQ ID NO: 70 - DNA, 253 bp, Caldalkalibacillus thermarum
tacgtagggggcaagcgttgtccggaatgattgggcgtaaagggcgcgtaggcggccatttaagtctgaagtgaaagtcctgctttcaaggtgggaactgctttggatactggatggcttgagtgcaggagaggtaagcggaattcccggtgtagcggtgaaatgcgtagagatcgggaggaacaccagtggcgaaggcggcttactggactgtaactgacgctgaggcgcgaaagtgtggggagcaaacagg

SEQ ID NO: 71 - DNA, 253 bp, Solitalea canadensis
tacggaggacccgagcgttatccggatttattgggtttaaagggtgcgtaggcgggcgattaagtcagaggtgaaagcccggggctcaactccggaactgcctttgatactggttgtcttgagtcatgacgaggatggcggaatgtgatgtgtagcggtgaaatgcatagatatgtcacagaacaccgattgcgaaggcagctgtctagagatgaactgacgctgaggcacgaaagtatggggatcaaacagg

SEQ ID NO: 72 - DNA, 253 bp, Anaerostipes caccae
tacgtatggtgcaagcgttatccggatttactgggtgtaaagggtgcgtaggtggtatggtaagtcagaagtgaaaacacggggctcaaccccgtgactgcttttgaaactatcaaactagagtccaggagaggtaagcggaattcctagtgtagcggtgaaatgcgtagatattaggaggaacaccagtggcgaaggcggcttactggactggtactgacactgaggcacgaaagcgtggggagcaaacagg

SEQ ID NO: 73 - DNA, 253 bp, Eisenbergiella massiliensis
tacgtagggggcaagcgttatccggatttactgggtgtaaagggagcgtagacggcatgacaagccagatgtgaaaacccagggctcaaccctgggactgcatttggaactgccaggctggagtgcaggagaggtaagcggaattcctagtgtagcggtgaaatgcgtagatattaggaggaacaccagtggcgaaggcggcttactggactgtaactgacgttgaggctcgaaagcgtggggagcaaacagg

SEQ ID NO: 74 - DNA, 252 bp, Olsenella profusa
tacgtagggggcaagcgttatccggattcattgggcgtaaagcgcgcgtaggcggccagcctggtcgggagtcaaatccgggggctcaacccccgtccgctcccgataccggccggcttgagtctggcaggggaaggcggaattcccggtgtagcggtggaatgcgcagatatcgggaagaacaccggtggcgaaggcggccttctgggccacgactgacgctgaggcgcgaaagctaggggagcgaacagg

SEQ ID NO: 75 - DNA, 253 bp, Dorea formicigenerans
tacgtagggggcaagcgttatccggatttactgggtgtaaagggagcgtagacggctatgcaagtctgaagtgaaaggcatgggctcaacctgtggactgctttggaaactgtgaagctagaatgtcggagaggcaaggggaattcctagtgtagcggtgaaatgcgtagatattaggaggaacaccagtggcgaaggcgccttgctggacgatgattgacgttgaggctcgaaagcgtggggagcaaacagg

SEQ ID NO: 76 - DNA, 253 bp, Blautia wexlerae
tacgtagggggcaagcgttatccggatttactgggtgtaaagggagcgtagacggaaggtcaagtcaggtgtgaaaggcatgggctcaacccgtggactgcacttgaaactggtcatctcgagtgtcggaggggcaggcggaattcctggtgtagcggtgaaatgcgtagatatcaggaagaacaccggtggcgaaggcggcctgctggacgatcactgacgttgaggctcgaaggcgtggggagcaaacagg

SEQ ID NO: 77 - DNA, 253 bp, [Eubacterium] rectale
tacgtatggtgcaagcgttatccggatttactgggtgtaaagggagcgtaggcggcttggcaagtctgatgtgaaaacccggggctcaacctcgggactgcattggaaactgtcgagctggagtgtcggagaggtaagcggaattcctagtgtagcggtgaaatgcgtagatattaggaggaacaccagtggcgaaggcggcttactggacgataactgacgctgaggctcgaaagcgtggggagcaaacagg

SEQ ID NO: 78 - DNA, 253 bp, Pseudomonas lini
tacagagggtgcaagcgttaatcggaattactgggcgtaaagcgcgcgtaggtggttcgttaagttggatgtgaaatccccgggctcaacctgggaactgcattcaaaactgacgagctagagtatggtagagggtggtggaatttcctgtgtagcggtgaaatgcgtagatataggaaggaacaccagtggcgaaggcgaccacctggactgatactgacactgaggtgcgaaagcgtggggagcaaacagg

SEQ ID NO: 79 - DNA, 253 bp, Prevotella shahii
tacggaaggttcgggcgttatccggatttattgggtttaaagggagcgtaggccgctgttcaagcgtgtcgtgaaatgcggttgctcaacgatcgcactgcggcgcgaactgtccggcttgagtcaagtgaaagtaggcggaattcgtggtgtagcggtgaaatgcttagatatcacgaggaactccgattgcgaaggcagcttactgtagttgtactgacgctgaagctcgaaggtgcgggtatcgaacagg

SEQ ID NO: 80 - DNA, 253 bp, Kroppenstedtia pulmonis
tacgtagggggcaagcgttgtccggaatgattgggcgtaaagggcgcgtaggcggcctgttaagtctggagtgaaactcccgcttttaaggtgggaactgctttggatactgatgggcttgagtgcaggagaggtaagcggaattcccggtgtagcggtgaaatgcgtagagatcgggaggaacaccagtggcgaaggcggcttactggactgtaactgacgctgaggcgcgaaagtgtggggagcaaacagg

SEQ ID NO: 81 - DNA, 253 bp, Geosporobacter ferrireducens
tacgtagggggcaagcgttgtccggaatgactgggcgtaaagggcgtgtaggcggccgattaagtctggagtgaaagtccacttttcaagggtggaattgctttggatactggttggcttgagtgcggaagaggtaagtggaattcccagtgtagcggtgaaatgcgtagagattgggaggaacaccagtggcgaaggcgacttactgggccgtaactgacgctgaggcgcgaaagcgtggggagcgaacagg

SEQ ID NO: 82 - DNA, 253 bp, Mediterranea massiliensis
tacggaagatgcgagcgttatccggatttattgggtttaaagggagcgtaggcgggtttttaagtcagctgtgaaagtttgaggctcaaccttaaaattgcagttgatactggagaccttgagtgcggtaaaggtaggcggaattcgtggtgtagcggtgaaatgcttagatatcacgaagaactccgattgcgaaggcagcttactgggccgtaactgacgctgatgctcgaaagtgcgggtatcaaacagg

SEQ ID NO: 83 - DNA, 253 bp, Staphylococcus aureus; identical to SEQ ID NO: 64

SEQ ID NO: 84 - DNA, 253 bp, Flavonifractor plautii
tacgtaggtggcgagcgttatccggatttactgggtgtaaagggtgtgtaggcgggatggcaagtcagatgtgaaatccgggggctcaacccccggcctgcatttgaaactgctgttcttgagtgtcggagaggtaaacggaattcccagtgtagcggtgaaatgcgtagatattgggaggaacaccagtggcgaaggcggtttactggacgacaactgacgctgagacacgaaagcgtggggagcaaacagg

SEQ ID NO: 85 - DNA, 252 bp, Butyricimonas virosa
tacgggggatgcgagcgttatccggatttattgggtttaaagggcgcgtaggcgggacgtcaagtcagcggtaaaagactgcagctaaactgtagcacgccgttgaaactggcgccctggagacgagacgagggaggcggaacaagtgaagtagcggtgaaatgcatagatatcacttggaaccccgatagcgaaggcagcttcccaggctcgttctgacgctgatgcgcgagagcgtgggtagcgaacagg

SEQ ID NO: 86 - DNA, 253 bp, Haemophilus sputorum
tacggggggtgcgagcgttaatcggaataactgggcgtaaagggcacgcaggcggtgacttaagtgagatgtgaaagccccgggcttaacctgggaattgcatttcatactgggtcgctagagtactttagggaggggtagaattccacgtgtagcggtgaaatgcgtagagatgtggaggaataccgaaggcgaaggcagccccttgggaatgtactgacgctcatgtgcgaaagcgtggggagcaaacagg